SEQUENCE LISTING (42 sequences)

SEQ ID NO: 1 - DNA, 2924 bp, Homo sapiens
  CDS (11)..(2311)
gacgccgacgatgaagacaccgtggaaggttcttctgggactgctgggtgctgctgcgcttgtcaccatcatcaccgtgcccgtggttctgctgaacaaaggcacagatgatgctacagctgacagtcgcaaaacttacactctaactgattacttaaaaaatacttatagactgaagttatactccttaagatggatttcagatcatgaatatctctacaaacaagaaaataatatcttggtattcaatgctgaatatggaaacagctcagttttcttggagaacagtacatttgatgagtttggacattctatcaatgattattcaatatctcctgatgggcagtttattctcttagaatacaactacgtgaagcaatggaggcattcctacacagcttcatatgacatttatgatttaaataaaaggcagctgattacagaagagaggattccaaacaacacacagtgggtcacatggtcaccagtgggtcataaattggcatatgtttggaacaatgacatttatgttaaaattgaaccaaatttaccaagttacagaatcacatggacggggaaagaagatataatatataatggaataactgactgggtttatgaagaggaagtcttcagtgcctactctgctctgtggtggtctccaaacggcacttttttagcatatgcccaatttaacgacacagaagtcccacttattgaatactccttctactctgatgagtcactgcagtacccaaagactgtacgggttccatatccaaaggcaggagctgtgaatccaactgtaaagttctttgttgtaaatacagactctctcagctcagtcaccaatgcaacttccatacaaatcactgctcctgcttctatgttgataggggatcactacttgtgtgatgtgacatgggcaacacaagaaagaatttctttgcagtggctcaggaggattcagaactattcggtcatggatatttgtgactatgatgaatccagtggaagatggaactgcttagtggcacggcaacacattgaaatgagtactactggctgggttggaagatttaggccttcagaacctcattttacccttgatggtaatagcttctacaagatcatcagcaatgaagaaggttacagacacatttgctatttccaaatagataaaaaagactgcacatttattacaaaaggcacctgggaagtcatcgggatagaagctctaaccagtgattatctatactacattagtaatgaatataaaggaatgccaggaggaaggaatctttataaaatccaacttagtgactatacaaaagtgacatgcctcagttgtgagctgaatccggaaaggtgtcagtactattctgtgtcattcagtaaagaggcgaagtattatcagctgagatgttccggtcctggtctgcccctctatactctacacagcagcgtgaatgataaagggctgagagtcctggaagacaattcagctttggataaaatgctgcagaatgtccagatgccctccaaaaaactggacttcattattttgaatgaaacaaaattttggtatcagatgatcttgcctcctcattttgataaatccaagaaatatcctctactattagatgtgtatgcaggcccatgtagtcaaaaagcagacactgtcttcagactgaactgggccacttaccttgcaagcacagaaaacattatagtagctagctttgatggcagaggaagtggttaccaaggagataagatcatgcatgcaatcaacagaagactgggaacatttgaagttgaagatcaaattgaagcagccagacaattttcaaaaatgggatttgtggacaacaaacgaattgcaatttggggctggtcatatggagggtacgtaacctcaatggtcctgggatcaggaagtggcgtgttcaagtgtggaatagccgtggcgcctgtatcccggtgggagtactatgactcagtgtacacagaacgttacatgggtctcccaactccagaagacaaccttgaccattacagaaattcaacagtcatgagcagagctgaaaattttaaacaagttgagtacctccttattcatggaacagcagatgataacgttcactttcagcagtcagctcagatctccaaagccctggtcgatgttggagtggatttccaggcaatgtggtatactgatgaagaccatggaatagctagcagcacagcacaccaacatatatatacccacatgagccacttcataaaacaatgtttctctttaccttagcacctcaaaataccatgccatttaaagcttattaaaactcatttttgttttcattatctcaaaactgcactgtcaagatgatgatgatctttaaaatacacactcaaatcaagaaacttaaggttacctttgttcccaaatttcatacctatcatcttaagtagggacttctgtcttcacaacagattattaccttacagaagtttgaattatccggtcgggttttattgtttaaaatcatttctgcatcagctgctgaaacaacaaataggaattgtttttatggaggctttgcatagattccctgagcaggattttaatctttttctaactggactggttcaaatgttgttctcttctttaaagggatggcaagatgtgggcagtgatgtcactagggcagggacaggataagagggattagggagagaagatagcagggcatggctgggaacccaagtccaagcataccaacacgaccaggctactgtcagctcccctcggagaaaactgtgcagtctgcgtgtgaacagctcttctcctttagagcacaatggatctcgagggatcttccatacctaccagttctgcgcctcgaggccgcgactctaga

SEQ ID NO: 2 - protein, 766 aa, Homo sapiens
MKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 3 - DNA, 3445 bp, Homo sapiens
  CDS (111)..(2411)
gagtgactccaccgcccggagcagcggtgcaggacgcgcgtctccgccgcccgcggtgacttctgcctgcgctccttctctgaacgctcacttccgaggagacgccgacgatgaagacaccgtggaaggttcttctgggactgctgggtgctgctgcgcttgtcaccatcatcaccgtgcccgtggttctgctgaacaaaggcacagatgatgctacagctgacagtcgcaaaacttacactctaactgattacttaaaaaatacttatagactgaagttatactccttaagatggatttcagatcatgaatatctctacaaacaagaaaataatatcttggtattcaatgctgaatatggaaacagctcagttttcttggagaacagtacatttgatgagtttggacattctatcaatgattattcaatatctcctgatgggcagtttattctcttagaatacaactacgtgaagcaatggaggcattcctacacagcttcatatgacatttatgatttaaataaaaggcagctgattacagaagagaggattccaaacaacacacagtgggtcacatggtcaccagtgggtcataaattggcatatgtttggaacaatgacatttatgttaaaattgaaccaaatttaccaagttacagaatcacatggacggggaaagaagatataatatataatggaataactgactgggtttatgaagaggaagtcttcagtgcctactctgctctgtggtggtctccaaacggcacttttttagcatatgcccaatttaacgacacagaagtcccacttattgaatactccttctactctgatgagtcactgcagtacccaaagactgtacgggttccatatccaaaggcaggagctgtgaatccaactgtaaagttctttgttgtaaatacagactctctcagctcagtcaccaatgcaacttccatacaaatcactgctcctgcttctatgttgataggggatcactacttgtgtgatgtgacatgggcaacacaagaaagaatttctttgcagtggctcaggaggattcagaactattcggtcatggatatttgtgactatgatgaatccagtggaagatggaactgcttagtggcacggcaacacattgaaatgagtactactggctgggttggaagatttaggccttcagaacctcattttacccttgatggtaatagcttctacaagatcatcagcaatgaagaaggttacagacacatttgctatttccaaatagataaaaaagactgcacatttattacaaaaggcacctgggaagtcatcgggatagaagctctaaccagtgattatctatactacattagtaatgaatataaaggaatgccaggaggaaggaatctttataaaatccaacttagtgactatacaaaagtgacatgcctcagttgtgagctgaatccggaaaggtgtcagtactattctgtgtcattcagtaaagaggcgaagtattatcagctgagatgttccggtcctggtctgcccctctatactctacacagcagcgtgaatgataaagggctgagagtcctggaagacaattcagctttggataaaatgctgcagaatgtccagatgccctccaaaaaactggacttcattattttgaatgaaacaaaattttggtatcagatgatcttgcctcctcattttgataaatccaagaaatatcctctactattagatgtgtatgcaggcccatgtagtcaaaaagcagacactgtcttcagactgaactgggccacttaccttgcaagcacagaaaacattatagtagctagctttgatggcagaggaagtggttaccaaggagataagatcatgcatgcaatcaacagaagactgggaacatttgaagttgaagatcaaattgaagcagccagacaattttcaaaaatgggatttgtggacaacaaacgaattgcaatttggggctggtcatatggagggtacgtaacctcaatggtcctgggatcgggaagtggcgtgttcaagtgtggaatagccgtggcgcctgtatcccggtgggagtactatgactcagtgtacacagaacgttacatgggtctcccaactccagaagacaaccttgaccattacagaaattcaacagtcatgagcagagctgaaaattttaaacaagttgagtacctccttattcatggaacagcagatgataacgttcactttcagcagtcagctcagatctccaaagccctggtcgatgttggagtggatttccaggcaatgtggtatactgatgaagaccatggaatagctagcagcacagcacaccaacatatatatacccacatgagccacttcataaaacaatgtttctctttaccttagcacctcaaaataccatgccatttaaagcttattaaaactcatttttgttttcattatctcaaaactgcactgtcaagatgatgatgatctttaaaatacacactcaaatcaagaaacttaaggttacctttgttcccaaatttcatacctatcatcttaagtagggacttctgtcttcacaacagattattaccttacagaagtttgaattatccggtcgggttttattgtttaaaatcatttctgcatcagctgctgaaacaacaaataggaattgtttttatggaggctttgcatagattccctgagcaggattttaatctttttctaactggactggttcaaatgttgttctcttctttaaagggatggcaagatgtgggcagtgatgtcactagggcagggacaggataagagggattagggagagaagatagcagggcatggctgggaacccaagtccaagcataccaacacgagcaggctactgtcagctcccctcggagaagagctgttcacagccagactggcacagttttctgagaaagactattcaaacagtctcaggaaatcaaatatgcaaagcactgacttctaagtaaaaccacagcagttgaaaagactccaaagaaatgtaagggaaactgccagcaacgcaggcccccaggtgccagttatggctataggtgctacaaaaacacagcaagggtgatgggaaagcattgtaaatgtgcttttaaaaaaaaatactgatgttcctagtgaaagaggcagcttgaaactgagatgtgaacacatcagcttgccctgttaaaagatgaaaatatttgtatcacaaatcttaacttgaaggagtccttgcatcaatttttcttatttcatttctttgagtgtcttaattaaaagaatattttaacttccttggactcattttaaaaaatggaacataaaatacaatgttatgtattattattcccattctacatactatggaatttctcccagtcatttaataaatgtgccttcattttttc

SEQ ID NO: 4 - protein, 766 aa, Homo sapiens; identical to SEQ ID NO: 2

SEQ ID NO: 5 - DNA, 340 bp, Homo sapiens
  exon (308)..(313)
cccccagtctcgggcccgactctgcccccgtgcgcccagcgccctacacgccctcagctcgcgggctcccccggccgggatgccagtgccgcgccacgcgcctcgtcccgccgcctgccctgcagcctgcccgcggcgcctttatacccagcggctcggcgctcactaatgtttaactcggggccgaaacttgccagccgagtgactccaccgcccggagcagcgtgcaggacgcgcgtctccgccgcccgcgtgacttctgcctgcgctccttctctgaacgctcacttccgaggagacgccgacgatgaaggtgagtgccgcgccagtacgtcccggg

SEQ ID NO: 6 - DNA, 240 bp, Homo sapiens
tgcctctggagcagccccgaccctctacggaaactggggccacacgcttccctctaattggacttgatctgctcggcttgcagacaccgtggaaggttcttctgggactgctgggtgctgctgcgcttgtcaccatcatcaccgtgcccgtggttctgctgaacaaaggcagtaagtttgaaacatttgaaaaagataggacagttggagctcaggtcaggctccgtcttaccacgctaa

SEQ ID NO: 7 - DNA, 527 bp, Homo sapiens
  exon (183)..(281)
gtaaatgcatgtttactccatccattcagtctgaagtcttttaaaatgtgtcttgcattttgtgagtatatttctgaattttttgagcagggtatattgtagagatccatttgtttattttatccaaaacttaggttattccatcttactgaaatgaacatttggctctttctttcaaccagcagatgatgctacagctgacagtcgcaaaacttacactctaactgattacttaaaaaatacttatagactgaagttatactccttaagatggatttcaggtaagaattttttggggtagggcagattttatgcttacattcattctagttaagtcgagatgtgggcttttatggcactgagaagaggtaatccttgtttttaaaaattctatagagagttttttttttctttttctacttgaagtgcaaagggagaaagactgaaaggaacccaagtatttctttgacatgtataataaaaattaaatgtgaattgaaaccttgacaataatgatgaaaaatgaa

SEQ ID NO: 8 - DNA, 423 bp, Homo sapiens
ttttctacttgaagtgcaaagggagaaagactgaaaggaacccaagtatttctttgacatgtataataaaaaattaaatgtgaattgaaaccttgacaataatgatgaaaaaatgaatgaatgttagtataatatcattcatgttttctcaacttagaaatgcctatttggtatttgtgagcttaaactgtattctgtaattttcagcttatgaagatgtttaataacaacttgaaattttgatctgggtgttttctctctttagatcatgaatatctctacaaacaagaaaataatatcttggtattcaatgctgaatatggaaacagctcagttttcttggagaacagtacatttgtatgtttaatcacgtaattaattcatgcatagaattctggggatttttttcatctatcctcttgg

SEQ ID NO: 9 - DNA, 555 bp, Homo sapiens
tttcccatgcctggatttgacgctattcccactgttgagaaagttttcttttcctcatcctcactctcgcaggcatctttcctcttaggattaatccttccctttctggctttctaacattgatcgccacctaccttgcatcacggtcatttgtgtgggtgaatatgccacctctgctagattaggacttcccaaagcacaagccacatgtctagaacagtaccccctatgaagtaagtgcacagtgatggccaatgaaatggaattgttcaatttttctttgttcttcttgtttgacaggatgagtttggacattctatcaatgattattcaatatctcctgatgggcagtttattctcttagaatacaactacgtgaaggtaaaataaatgctttcttactgtaacatttaaataatccaagagtatgctatgttataaccgatttcaagtgtatggatacttaagattaatatctaagtaatgtaataggcactaaaactcattaattcagaatcagagtcattctcattcttaaagacaagttcagtgttt

SEQ ID NO: 10 - DNA, 310 bp, Homo sapiens
ctaggatacttctgaatcccaaaattctcggaggaaattataagatatgataacatgtgagaattatacaatcatatattaccaaacctatcatcttggtctcctatagtgagtggccaaattatcaagtatcacagaagcctgaaatgtctgttttctttcatagcaatggaggcattcctacacagcttcatatgacatttatgatttaaataaaaggtcagtgacttccaataaaatctattaggctttcctactgtcatatttttaaggaaaccccaatgtgagttattctttttgataatatttc

SEQ ID NO: 11 - DNA, 324 bp, Homo sapiens
cccaataaaatctattaggctttcctactgtcatatttttaaggaaaccccaatgtgagttattctttttgataatatttcctttcatcttttttccaggcagctgattacagaagagaggattccaaacaacacacagtgggtcacatggtcaccagtgggtcataaattggttagtgagtctctccagttgtcagaaaaatataggctcaaatttttcataggcagaagttactaaccctgatacaaagcagacatcttaggaactcaatacttcactatagaacatagtgttattgcccaggatctaatgtttgtctgcag

SEQ ID NO: 12 - DNA, 462 bp, Homo sapiens
atatttgactatggtaatccttacatttgcaatagataagtgtttctacaattccgggataaaaatatgacatgtattatagatatcaatgcttgttttttttccttctaggcatatgtttggaacaatgacatttatgttaaaattgaaccaaatttaccaagttacagaatcacatggacggggaaagaagatataatatataatggaataactgactgggtttatgaaggtagaaactttaaagctttttcaaatcagaaaatctataagcttttaaaatagttaaatgtttcttttgcaatgggaaaatttaaatgagattgaaaaaaagtttcattgtttaaaatgtcatcagatttctcttagaactcacagcaccagatgtctccttgaccaagaatttaactattcctagcactcctctgtcctcattgcctgacaatactcctcttgtctctg

SEQ ID NO: 13 - DNA, 471 bp, Homo sapiens
aagtggttcattagttgaactctactgtaattactaaagctaatgagaacaatattttgaatgccaaagccaatttatcagctacttcttgtaacagaacaatttatgtcttttttcagaggaagtcttcagtgcctactctgctctgtggtggtctccaaacggcacttttttagcatatgcccaatttaacgacacagaagtcccacttattgaatactccttctactctgatgagtcactgcagtacccaaagactgtacgggttccatatccaaaggtctgtgctcctgtttgtacagtggttccatgatttgatgggaagagaatggtttcatttaatcccttcttgctaatgaaaatattgtcagtctctcttcaacagcttgcgttgattactcttgctgaagtcaacagcatctggcaattttaaagtatttttattatcccaggaaagagtgtgatttaagg

SEQ ID NO: 14 - DNA, 327 bp, Homo sapiens
catccatcgtgtatgtttttcctaggcaggagctgtgaatccaactgtaaagttctttgttgtaaatacagactctctcagctcagtcaccaatgcaacttccatacaaatcactgctcctgcttctatgttgatagggtaagactcttgtcctgaatgcttggcttgtaaacagttttacactttctagggagaacttttcaaaagtgaatggcaaagtggatcacagcctcccactgtgtcatccccgctgacagtcattcattctttccttcagcaactctttgttgagcatcttcacgtcctcaggctgggcacaactctaga

SEQ ID NO: 15 - DNA, 246 bp, Homo sapiens
aaaatgtctattgtaccaaggtctggcaatagtcatgttcattctctctctctgtccttcctttctttcttccttccagggatcactacttgtgtgatgtgacatgggcaacacaagaaagaatttctttgcagtggctcaggaggattcagaactattcggtcatggatatttgtgactatgatgaatccagtggaagatggaactgcttagtggtgagactgaatggggatgttctgcttctag

SEQ ID NO: 16 - DNA, 513 bp, Homo sapiens
aagagagtcaaatgatctaatgtaatgacttgaaaagtactaacataaatagaaatatatgaatggagatagctctcagttattataagtacagctctatttatattaccacaaattatggctatagatttcatctgacttctctttgaaatgctgtcattgtctctttcccttcctaggcacggcaacacattgaaatgagtactactggctgggttggaagagtaagtgttataagttgtggggttttatggatgagtcagctactcagctatttgatatgcagctatttaaaactagggctctaagtgatacgttattaatagcaatattagttaacctatttctcaaaatatgtaaatgctccctataaagatatctttttgtagagaatatataacacacacagttggaaaaagtaaggggacaagtgaaaaatcaaaagcagagcagacaaaacatagcgcactcctggcaagttgtaactcccagcaagttaaagggtaccatggc

SEQ ID NO: 17 - DNA, 523 bp, Homo sapiens
gatgtctccaaatcacaacaactctatttattgtttctacaagcctctttatatacatctctcatttactgctcacgaaaattacctatgagtatggtgtttttgttcacattttatagaaagataattttctttttgagacactaattttaatttggcatataaatggagcatggatttttgagcgtaggtattggacaggactttctaaaactaaagttggtactgagatgagacactgtgctttaatgttcttttgttctctattattttctagtttaggccttcagaacctcattttacccttgatggtaatagcttctacaagatcatcagcaatgaagaaggttacagacacatttgctatttccaaatagataaaaaagtgagtattcttacatatattttatttcatgttgacctcttttttttttttggaaaccaacttgtagtggaaatcatttggtaaggtggatcaatgtgtataaaaatcagattaatttaaactcctcagtggtgtatt

SEQ ID NO: 18 - DNA, 349 bp, Homo sapiens
atttgaattccatgatataaatgatttaatcctccactttgaacagagagagtctctaatcaatctttttttttttttgaaaaaaaaaaaaaaaccatcaggactgcacatttattacaaaaggcacctgggaagtcatcgggatagaagctctaaccagtgattatctgtgagtattgtaacaattgataggatataaacctacttgtcataatcatgccctctttgcccagaagtagggaaaaaatctcctacaaataaagagtagtctttttggaatgtgttcaatactttttttctttttaacatacggaagccttaatgattttcacatatgcttagcagattt

SEQ ID NO: 19 - DNA, 574 bp, Homo sapiens
gggtctcaatttcaggctagctgcctagtcctatttttttttgacagaagccttcttttgtgtttttacttgtcactctaattaaggaagaaatctctggttttacatattttagtctgttaagaacaagacctttatgtgtagagatgacggagaaaaatggcttctcgggggtgagggacacggaagcgatggcggagggcggcgggcacccgtgcgcgcgctccgtgcacacttaggcttggggtctgagtgcggtggatcgcctctgacttcttaaaaaacataattaatatatatttcatttttttctcttcctagatactacattagtaatgaatataaaggaatgccaggaggaaggaatctttataagtaagagctctgtcaagttgttgacttgagtcattttagctgacgaacaatttctgaactctatttttattttttgccttgtcctaactaatattaaactggtattgctattgagcactttttattccttcataaatcaatctacatgtccacaagagagaatggctttattcgatgttttgtgatatttccaaacaat

SEQ ID NO: 20 - DNA, 309 bp, Homo sapiens
ctagaatatggctttattcagttccttcctctcacttatcttttcttcaagcacttaatttccaccgttggttttacagaatccaacttagtgactatacaaaagtgacatgcctcagttgtgagctgaatccggaaaggtgtcagtactattctgtgtcattcagtaaagaggcgaagtattatcagctgagatgttccggtaagtctgagctccctgggagggagggcagtcgcgctgttactcacttcgaagcagctccctctattccttttgaatctgaggagctcttgaaatcacctaagtccc

SEQ ID NO: 21 - DNA, 587 bp, Homo sapiens
ttagagtgggaaggatgtcagctcacattttactttgcagatgagtatattaagttcagagatgataatgtgcccaaggtccagcagggcacagattcatttcctacttcattctactgagccctgtgcctttccagtaatacttcttacactgaaattgcaactattttaaagagagggggaggagaaacagctaacatgtattaaacttcttatgcattataatatttatatatattctatcaaagttaaatggtaacaaatggtatttttgaaaagtcatctatactatcttttctaaaaaagtattcatccaaaacatttttcccccattgtgttaacaggtcctggtctgcccctctatactctacacagcagcgtgaatgataaaggtattttctcatattttactcttcatttgtttctaagaaagtgtgttgcataagtctaacatgtacttagcattgtgtgattttctttggctttgtgtcttgacaatgagttgaacacaagccttataacctgaagtatgtgtagtccgatcagtgaatttcatgtttgctcattattgcactacataatccaca

SEQ ID NO: 22 - DNA, 569 bp, Homo sapiens
ccccattgtgttaacaggtcctggtctgcccctctatactctacacagcagcgtgaatgataaaggtattttctcatattttactcttcatttgtttctaagaaagtgtgttgcataagtctaacatgtacttagcattgtgtgattttctttggctttgtgtcttgacaatgagttgaacacaagccttataacctgaagtatgtgtagtccgatcagtgaatttcatgtttgctcattattgcactacataatccacatttttcacattgtaatgaatgactgctgtctcctgtatttctttagggctgagagtcctggaagacaattcagctttggataaaatgctgcagaatgtccagatgccctccaaaaaactggacttcattattttgaatgaaacaagtaagttaaattcctttggaatagtacttttcatggaaacaccaatgcactctaactatagtaattcgatctgaaaatatgtatttcatgttatatataaatctaatttaaaaggttatttctccatgaaatgttttgtcacaagaaatactaattttacatcc

SEQ ID NO: 23 - DNA, 256 bp, Homo sapiens
tctagaaatgacttgctactgacggacatgaggaacaaaatcttgtgctaataatttttcttacctcactaattaatctttctgtttgtttagaattttggtatcagatgatcttgcctcctcattttgataaatccaagaaatatcctctactattagatgtgtaagtattcaagagaagggagcaatttagaagtattgagtcatatatatttttctcagtgcagcttactgaaaaatggagccaaatgcgtcc

SEQ ID NO: 24 - DNA, 334 bp, Homo sapiens
attcctcatttgattccagaaaaccatgaaagctaacaatttggtatttttcataggtatgcaggcccatgtagtcaaaaagcagacactgtcttcagactgaactgggccacttaccttgcaagcacagaaaacattatagtagctagctttgatggcagaggaagtggttaccaaggagataagatcatgcatgcaatcaacagaagactgggaacatttgaagttgaagatcaaattgaagcagccaggtgagtgactaggagatgaagtcaaaggtcatctgattttttttcccctttaaaatttacattttaaagctgtaaagtgtaaa

SEQ ID NO: 25 - DNA, 724 bp, Homo sapiens
ggggcagactctcccaaactgaaaaacatctggagagatttaaattcaattatgtaaagaaaagtgcatctgggctgcaaatttttgtataaaactgtgaaaaaaactgccaggatttgttttctgtgaatcatacacacacacacacacacacacacacacacacacacacacacaatttttatgaactccaacaaaagtaaccatgtcatctggatcttaggttaaaatgatagatatttatctcaggtgtttatttgattactggcagcaatttttagtctctaatagaatgaagcagagaacaaatggcagggtttcacatttgtgcctcatctaagttttctatacacaatgcagtactgatctgtggactttttccttgtgttttagacaattttcaaaaatgggatttgtggacaacaaacgaattgcaatttggggctgggtgagtagttgactgatgaatctgtatgcattcgttagtgaatatcactcagagcaggttactttatcttttgtatatacatatttggaggtgaaaatcacatctatcaagtcttaggtctctgggcagtaactctgcagaatctccactgagttaggcttacacctttgtttattatgatgagccagggaggtgaggtagaaagagcaagaaggaaggagtgaaagacgggagggaagagggaagggaaggaaggccagggagactttttcttat

SEQ ID NO: 26 - DNA, 724 bp, Homo sapiens
caattaaggccaatttaagcctcctataccatgctttttatctctaaagaggcacagtatataaagatgtgagaaatgtggaaactgcgactcgcttaccatatgtccttattcttttacctccataaaaataactgtttccattccttagtcatatggagggtacgtaacctcaatggtcctgggatcgggaagtggcgtgttcaagtgtggaatagccgtggcgcctgtatcccggtgggagtactatggtatgtaattgctgccaggtacaaagagatacggagtgttgacctcaaaatgccagtaacaaagccattcctttctgagtttcagtgtttttacagcgaataataggctgattcatcttagaggttctttttggatacctataataagatgatctcatttattctgctattttgcaaggctgggtggtatattttattacacgtttgattttttatcaacaaattctgttttccgaatttcaaaatagccccatgctacatttctacagataaatcattccttatctttactctgatttgattcttgggtttcacatattagcagagtgagagaagtatgtatctctttcccatcaattaaatgtcaaatacaagtagggctttggatgggaagaggagaggaggaacagtggaaacacccattggggggttgttgttttttttcattcttaataggtgaaagctactctaga

SEQ ID NO: 27 - DNA, 264 bp, Homo sapiens
gtaactctatattcctatacatgagtgaatacatatggaaacactgaagtggcccattaaaagcaagaaaagttgttattgtatgaattaaacttttttttatttttttttctttcacagactcagtgtacacagaacgttacatgggtctcccaactccagaagacaaccttgaccattacagagtaagagatgagacctatacctattttataacctatttataagtggtttaagaaccttcttaattgtatctattctaga

SEQ ID NO: 28 - DNA, 348 bp, Homo sapiens
tgtctcccaggcacagggctgctgtgcattctgccatgcatggagacagcatttgcttgaatatcggtttgctatattttgtatcaaagggggccccagctgaaaacattttattttgtaaactgaagtgaaaaatcagtttatggcagatccttttttgaataatgacattctttctcccaattccagaattcaacagtcatgagcagagctgaaaattttaaacaagttgagtacctccttattcatggaacagcagatggtgagtttcagttaattagactttttagtatcacagacaggtttgcaatttcactactgacaaaacaaaagcatgaggggcaagac

SEQ ID NO: 29 - DNA, 406 bp, Homo sapiens
atatttaccaaagttgggatgaaggtaaaaatgccataaaatactgtagtgcctttgggagggggtgtagctagagtctactttgatggtctgttatgtgttgggcaggaaaggggcagcagcatctccatgggtttaaaagcagccagctcgtcctgcagataacgttcactttcagcagtcagctcagatctccaaagccctggtcgatgttggagtggatttccaggcaatggtatagaaaaactttgtgcaaggggagggaagttgctggtggggtctagtttcctctttctgggctaattcttttagcagtgccacagacaggacagaataaaaacattaaaaatttttttttcaacgtgaagtcagaagagtggtatccttcagccatgcacagagtaat

SEQ ID NO: 30 - DNA, 1665 bp, Homo sapiens
ctgcaggacagaacaacgtgccctggaaaagctctgggggaaaattagaccccctctttgaccccgtttcagtttataaactgcctcaggaattaaaacactctgttttataccatgtatgctcgtgagagctcatttgaatttacgtatttttttttttaagaagagtactgacttcctaattctaacttcttttctttctctctctagtggtatactgatgaagaccatggaatagctagcagcacagcacaccaacatatatatacccacatgagccacttcataaaacaatgtttctctttaccttagcacctcaaaataccatgccatttaaagcttattaaaactcatttttgttttcattatctcaaaactgcactgtcaagatgatgatgatctttaaaatacacactcaaatcaagaaacttaaggttacctttgttcccaaatttcatacctatcatcttaagtagggacttctgtcttcacaacagattattaccttacagaagtttgaattatccggtcgggttttattgtttaaaatcatttctgcatcagctgctgaaacaacaaataggaattgtttttatggaggctttgcatagattccctgagcaggattttaatctttttctaactggactggttcaaatgttgttctcttctttaaagggatggcaagatgtgggcagtgatgtcactagggcagggacaggataagagggattagggagagaagatagcagggcatggctgggaacccaagtccaagcataccaacacgagcaggctactgtcagctcccctcggagaagagctgttcacagccagactggcacagttttctgagaaagactattcaaacagtctcaggaaatcaaatatgcaaagcactgacttctaagtaaaaccacagcagttgaaaagactccaaagaaatgtaagggaaactgccagcaacgcaggcccccaggtgccagttatggctataggtgctacaaaaacacagcaagggtgatgggaaagcattgtaaatgtgcttttaaaaaaaaatactgatgttcctagtgaaagaggcagcttgaaactgagatgtgaacacatcagcttgccctgttaaaagatgaaaatatttgtatcacaaatcttaacttgaaggagtccttgcatcaatttttcttatttcatttctttgagtgtcttaattaaaagaatattttaacttccttggactcattttaaaaaatggaacataaaatacaatgttatgtattattattcccattctacatactatggaatttctcccagtcatttaataaatgtgccttcattttttcagaagtaatgacttgagtcttgaataagacctaaaccgcactggcatatggacacatttatctagaaaggctcttgtttattttgttagtaatcccagactaccttgtaaacatggaaagagttcagctttggtaaaaaaattagatatataaaaatgtgttaagctaaacagttcataggtttttgcctaataatgtgcaatttctcttatcataaatacatacagcttttgagaaatcaacttaaactaattttagttacttatatcatacacaatttataattttattccagggaactatgatgaggcttatataa

SEQ ID NO: 31 - DNA, 1665 bp, Homo sapiens; identical to SEQ ID NO: 30

SEQ ID NO: 32 - DNA, 3410 bp, Homo sapiens
  CDS (77)..(2143)
cgcgcgtctccgccgcccgcggtgacttctgcctgcgctccttctctgaacgctcacttccgaggagacgccgacgatgaagacaccgtggaaggttcttctgggactgctgggtgctgctgcgcttgtcaccatcatcaccgtgcccgtggttctgctgaacaaaggcacagatgatgctacagctgacagtcgcaaaacttacactctaactgattacttaaaaaatacttatagactgaagttatactccttaagatggatttcagatcatgaatatctctacaaacaagaaaataatatcttggtattcaatgctgaatatggaaacagctcagttttcttggagaacagtacatttgatgagtttggacattctatcaatgattattcaatatctcctgatgggcagtttattctcttagaatacaactacgtgaagcaatggaggcattcctacacagcttcatatgacatttatgatttaaataaaaggcagctgattacagaagagaggattccaaacaacacacagtgggtcacatggtcaccagtgggtcataaattggcatatgtttggaacaatgacatttatgttaaaattgaaccaaatttaccaagttacagaatcacatggacggggaaagaagatataatatataatggaataactgactgggtttatgaagaggaagtcttcagtgcctactctgctctgtggtggtctccaaacggcacttttttagcatatgcccaatttaacgacacagaagtcccacttattgaatactccttctactctgatgagtcactgcagtacccaaagactgtacgggttccatatccaaaggcaggagctgtgaatccaactgtaaagttctttgttgtaaatacagactctctcagctcagtcaccaatgcaacttccatacaaatcactgctcctgcttctatgttgataggggatcactacttgtgtgatgtgacatgggcaacacaagaaagaatttctttgcagtggctcaggaggattcagaactattcggtcatggatatttgtgactatgatgaatccagtggaagatggaactgcttagtggcacggcaacacattgaaatgagtactactggctgggttggaagatttaggccttcagaacctcattttacccttgatggtaatagcttctacaagatcatcagcaatgaagaaggttacagacacatttgctatttccaaatagataaaaaagactgcacatttattacaaaaggcacctgggaagtcatcgggatagaagctctaaccagtgattatctatactacattagtaatgaatataaaggaatgccaggaggaaggaatctttataaaatccaacttagtgactatacaaaagtgacatgcctcagttgtgagctgaatccggaaaggtgtcagtactattctgtgtcattcagtaaagaggcgaagtattatcagctgagatgttccggtcctggtctgcccctctatactctacacagcagcgtgaatgataaagggctgagagtcctggaagacaattcagctttggataaaatgctgcagaatgtccagatgccctccaaaaaactggacttcattattttgaatgaaacaaaattttggtatcagatgatcttgcctcctcattttgataaatccaagaaatatcctctactattagatgtgtatgcaggcccatgtagtcaaaaagcagacactgtcttcagactgaactgggccacttaccttgcaagcacagaaaacattatagtagctagctttgatggcagaggaagtggttaccaaggagataagatcatgcatgcaatcaacagaagactgggaacatttgaagttgaagatcaaattgaagcagccagacaattttcaaaaatgggatttgtggacaacaaacgaattgcaatttggggctggtcatatggagggtacgtaacctcaatggtcctgggatcgggaagtggcgtgttcaagtgtggaatagccgtggcgcctgtatcccggtgggagtactatgactcagtgtacacagaacgttacatgggtctcccaactccagaagacaaccttgaccattacagaattcaacagtcatgagcagagctgaaaattttaaacaagttgagtacctccttattcatggaacagcagatgataacgttcactttcagcagtcagctcagatctccaaagccctggtcgatgttggagtggatttccaggcaatgtggtatactgatgaagaccatggaatagctagcagcacagcacaccaacatatatatacccacatgagccacttcataaaacaatgtttctctttaccttagcacctcaaaataccatgccatttaaagcttattaaaactcatttttgttttcattatctcaaaactgcactgtcaagatgatgatgatctttaaaatacacactcaaatcaagaaacttaaggttacctttgttcccaaatttcatacctatcatcttaagtagggacttctgtcttcacaacagattattaccttacagaagtttgaattatccggtcgggttttattgtttaaaatcatttctgcatcagctgctgaaacaacaaataggaattgtttttatggaggctttgcatagattccctgagcaggattttaatctttttctaactggactggttcaaatgttgttctcttctttaaagggatggcaagatgtgggcagtgatgtcactagggcagggacaggataagagggattagggagagaagatagcagggcatggctgggaacccaagtccaagcataccaacacgagcaggctactgtcagctcccctcggagaagagctgttcacagccagactggcacagttttctgagaaagactattcaaacagtctcaggaaatcaaatatgcaaagcactgacttctaagtaaaaccacagcagttgaaaagactccaaagaaatgtaagggaaactgccagcaacgcaggcccccaggtgccagttatggctataggtgctacaaaaacacagcaagggtgatgggaaagcattgtaaatgtgcttttaaaaaaaaatactgatgttcctagtgaaagaggcagcttgaaactgagatgtgaacacatcagcttgccctgttaaaagatgaaaatatttgtatcacaaatcttaacttgaaggagtccttgcatcaatttttcttatttcatttctttgagtgtcttaattaaaagaatattttaacttccttggactcattttaaaaaatggaacataaaatacaatgttatgtattattattcccattctacatactatggaatttctcccagtcatttaataaatgtgccttcattttttc

SEQ ID NO: 33 - protein, 688 aa, Homo sapiens
MKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRIQQS

SEQ ID NO: 34 - DNA, 2461 bp, Homo sapiens
  CDS (26)..(2326)
ggggacttccgaggagacgccgacgatgaagacaccgtggaaggttctcctgggactgctgggtgctgctgcgcttgtcaccatcatcaccgtgcccgtggttctgctgaacaaaggcacagatgatgctacagctgacagtcgcaaaacttacactctaactgattacttaaaaaatacttatagactgaagttatactccttaagatggatttcagatcatgaatatctctacaaacaagaaaataatatcttggtattcaatgctgaatatggaaacagctcagttttcttggagaacagtacatttgatgagtttggacattctatcaatgattattcaatatctcctgatgggcagtttattctcttagaatacaactacgtgaagcaatggaggcattcctacacagcttcatatgacatttatgatttaaataaaaggcagctgattacagaagagaggattccaaacaacacacagtgggtcacatggtcaccagtgggtcataaattggcatatgtttggaacaatgacatttatgttaaaattgaaccaaatttaccaagttacagaatcacatggacggggaaagaagatataatatataatggaataactgactgggtttatgaagaggaagtcttcagtgcctactctgctctgtggtggtctccaaacggcacttttttagcatatgcccaatttaacgacacagaagtcccacttattgaatactccttctactctgatgagtcactgcagtacccaaagactgtacgggttccatatccaaaggcaggagctgtgaatccaactgtaaagttctttgttgtaaatacagactctctcagctcagtcaccaatgcaacttccatacaaatcactgctcctgcttctatgttgataggggatcactacttgtgtgatgtgacatgggcaacacaagaaagaatttctttgcagtggctcaggaggattcagaactattcggtcatggatatttgtgactatgatgaatccagtggaagatggaactgcttagtggcacggcaacacattgaaatgagtactactggctgggttggaagatttaggccttcagaacctcattttacccttgatggtaatagcttctacaagatcatcagcaatgaagaaggttacagacacatttgctatttccaaatagataaaaaagactgcacatttattacaaaaggcacctgggaagtcatcgggatagaagctctaaccagtgattatctatactacattagtaatgaatataaaggaatgccaggaggaaggaatctttataaaatccaacttagtgactatacaaaagtgacatgcctcagttgtgagctgaatccggaaaggtgtcagtactattctgtgtcattcagtaaagaggcgaagtattatcagctgagatgttccggtcctggtctgcccctctatactctacacagcagcgtgaatgataaagggctgagagtcctggaagacaattcagctttggataaaatgctgcagaatgtccagatgccctccaaaaaactggacttcattattttgaatgaaacaaaattttggtatcagatgatcttgcctcctcattttgataaatccaagaaatatcctctactattagatgtgtatgcaggcccatgtagtcaaaaagcagacactgtcttcagactgaactgggccacttaccttgcaagcacagaaaacattatagtagctagctttgatggcagaggaagtggttaccaaggagataagatcatgcatgcaatcaacagaagactgggaacatttgaagttgaagatcaaattgaagcagccagacaattttcaaaaatgggatttgtggacaacaaacgaattgcaatttggggctggtcatatggagggtacgtaacctcaatggtcctgggatcgggaagtggcgtgttcaagtgtggaatagccgtggcgcctgtatcccggtgggagtactatgactcagtgtacacagaacgttacatgggtctcccaactccagaagacaaccttgaccattacagaaattcaacagtcatgagcagagctgaaaattttaaacaagttgagtacctccttattcatggaacagcagatgataacgttcactttcagcagtcagctcagatctccaaagccctggtcgatgttggagtggatttccaggcaatgtggtatactgatgaagaccatggaatagctagcagcacagcacaccaacatatatatacccacatgagccacttcataaaacaatgtttctctttaccttagcacctcaaaataccatgccatttaaagcttattaaaactcatttttgttttcattatctcaaaactgcactgtcaagatgatgatgatctttaaaatacacactcaaaaaaaaaaaaaaaaaaaaaaaaaaaaaa

SEQ ID NO: 35 - protein, 766 aa, Homo sapiens; identical to SEQ ID NO: 2

SEQ ID NO: 36 - DNA, 3316 bp, Mus musculus
  CDS (118)..(2400)
ggcggcgagctcagggtgactgcgtgcagagcagccgcgcaggacgtccgtctctgcgcgcagtgacttctgcctgcgctcaagcttcagagttcagtttcaaggagccgcccgaccatgaagacaccgtggaaggttcttctgggactgcttggtgtcgctgcgcttgtcaccatcatcaccgtgccaatagttctgctgagcaaagatgaagcggcagctgacagccgcagaacgtattcactagctgactatttaaagagtacctttcgggtcaagtcctactctttgtggtgggtttcagactttgaatacctctacaaacaagagaacaatatcttgctgctcaatgctgaacatggaaacagctccattttcttggagaacagtacctttgaaagctttggatatcattcagtgtcacctgaccgactgtttgttctcttggaatacaactacgtgaagcaatggagacattcctacacagcttcatacaacatttatgatgtgaataaaagacagctgatcacagaagagaagattccaaataatacacagtggatcacatggtcaccagaaggtcataagttggcatatgtctggaagaatgatatttacgttaaagttgaaccacacttacctagtcataggatcacatcgacaggagaagaaaatgtaatatataatggaataactgactgggtttatgaagaggaagtcttcggtgcctactctgcactgtggtggtctccaaacaacacgtttctagcttatgcccagtttaacgacacaggagtgccgctcattgaatactccttctattctgatgagtcactgcagtaccccaagacagtgtggattccatacccaaaggcaggagctgtgaatccaactgtaaagttctttattgtaaatatagactctctcagctcatcctctagtgcggctcccatccaaatccctgctcctgcatctgtggcaagaggggatcactatttatgtgatgtggtgtgggctacagaagaaagaatttcactacagtggctcaggaggattcagaactattccgtgatggctatctgtgactatgataagatcaacctaacgtggaactgtccatccgagcagcagcatgttgaaatgagtaccacaggctgggtcggaagatttaggcccgcagaacctcacttcacctctgatggaagcagcttctataagatcatcagcgacaaagatggctacaaacacatctgccacttcccgaaagataagaaagactgtacatttattacaaaaggagcctgggaagtcattagtatcgaagctctgaccagcgattatctatactacattagtaaccaatataaagaaatgccaggaggaagaaatctctataaaattcaacttactgaccacacaaatgtgaagtgccttagttgtgacctgaatccagaaagatgtcagtattatgcggtatcatttagtaaagaggcaaagtactatcagctgggatgttggggccccggtctgcccctctacactctacatcgtagcacggatcataaagagctgcgagtcctggaagacaattctgctttggatagaatgctgcaggatgtccagatgccttcaaaaaaattggacttcattgttttgaatgaaacaagattttggtatcaaatgatcttgccccctcattttgataaatccaagaaatatcctctactattagatgtatatgcaggtccctgtagtcaaaaagcagatgcttccttcagactgaactgggccacttaccttgcaagtacagaaaacatcatagtagctagctttgacggcagaggaagtggttaccaaggagataagatcatgcatgcaatcaacagaagattgggaacactggaagttgaagatcaaattgaagcagccaggcaatttgtaaaaatgggatttgtggatagcaagcgagttgcaatttggggctggtcatatggagggtatgtaacctcaatggtcctgggatcgggaagtggcgtgttcaagtgcggaatagctgtggcacctgtgtcacggtgggagtactatgactcagtgtacacagagcgttacatgggtctcccaattccagaagacaaccttgaccattacaggaactcaacagtcatgagcagagctgaacattttaaacaagttgagtacctccttattcatggaacggcagatgataatgttcactttcagcagtcagctcagatctccaaagccctggtggatgctggtgtggatttccaagcaatgtggtacacggatgaagaccacgggatcgctagcagcacagctcaccagcacatctattcccacatgagccatttcctccagcagtgcttctccttacactagcacagcatagctctccatagcttatttaagaccacatttgttctcattatctcaaaagtgcactgttaagatgaagatcttaataatgttgcattgagacatttcaggctgctttctccagttttacacctgcaatcctaactaaggatgcctgtccccagaacagattactaccttagagcaatttggattttcccctctgttttgtttatcatttaaaatcatttccacatcagctgcctgaaacaacaaatataaattatttttgccaagagctatgcatagatttcctgagcagaatttcaattttttttttccttactagactggaccaaatctcgtttcccttatttaagtgggtgacaagatgtgggtaatgattcattagggcagcaacaagagaagagggagcagggagtatggctagaaacccaggtccaagcatacaaaccaaccaggctactgtcagctcgcctcgagaagagctgttcactgccagactggcacagttttctgagaaagactattcaaacagtctcaggaaatcatatatgcaaagcactgacttctaagtaaaaccacagcagttgaatagactccaaagaaatgcaagggacgctgccagcaatgtaagggccccaggtgccagttatgcgtataggtgctacaaaaacacagcaagggtgatgggaaagcattgtaaatgtcttttgaaaattaccgatgtctcctagtgaaaagaggcagcttggaacatatcgacttgccctgttaaaagttgaaaatatttgtgtcacaaattctaacatgaaggaatacttgcattagtttttcctacttcatttctttgagcattttaattaaaatattttaacttcattagctttttaatggaaaattaaaaaaaaaaaaaaaa

SEQ ID NO: 37 - protein, 760 aa, Mus musculus
MKTPWKVLLGLLGVAALVTIITVPIVLLSKDEAAADSRRTYSLADYLKSTFRVKSYSLWWVSDFEYLYKQENNILLLNAEHGNSSIFLENSTFESFGYHSVSPDRLFVLLEYNYVKQWRHSYTASYNIYDVNKRQLITEEKIPNNTQWITWSPEGHKLAYVWKNDIYVKVEPHLPSHRITSTGEENVIYNGITDWVYEEEVFGAYSALWWSPNNTFLAYAQFNDTGVPLIEYSFYSDESLQYPKTVWIPYPKAGAVNPTVKFFIVNIDSLSSSSSAAPIQIPAPASVARGDHYLCDVVWATEERISLQWLRRIQNYSVMAICDYDKINLTWNCPSEQQHVEMSTTGWVGRFRPAEPHFTSDGSSFYKIISDKDGYKHICHFPKDKKDCTFITKGAWEVISIEALTSDYLYYISNQYKEMPGGRNLYKIQLTDHTNVKCLSCDLNPERCQYYAVSFSKEAKYYQLGCWGPGLPLYTLHRSTDHKELRVLEDNSALDRMLQDVQMPSKKLDFIVLNETRFWYQMILPPHFDKSKKYPLLLDVYAGPCSQKADASFRLNWATYLASTENIIVASFDGRGSGYQGDKIMHAINRRLGTLEVEDQIEAARQFVKMGFVDSKRVAIWGWSYGGYVTSMVLGSGSGVFKCGIAVAPVSRWEYYDSVYTERYMGLPIPEDNLDHYRNSTVMSRAEHFKQVEYLLIHGTADDNVHFQQSAQISKALVDAGVDFQAMWYTDEDHGIASSTAHQHIYSHMSHFLQQCFSLH

SEQ ID NO: 38 - DNA, 21 bp, Homo sapiens
caagagatggccacggctgct

SEQ ID NO: 39 - DNA, 21 bp, Homo sapiens
tccttctgcatcctgtcggca

SEQ ID NO: 40 - DNA, 34 bp, Homo sapiens
atgggactgagtaacattctctttgtgatggcct

SEQ ID NO: 41 - DNA, 36 bp, Homo sapiens
ctcgagttaaaaacatgtatcacttttgtcgcatga

SEQ ID NO: 42 - protein, 5 aa, Artificial Sequence
  Description of Artificial Sequence  Synthetic Peptide
GWSYG

(19) United States
(12) Patent Application Publication
(10) Pub. No.: US 2006/0093553 A1
(43) Pub. Date: May 4, 2006

(54) Cd26-based therapies for cancers and immune disease

(76) Inventors: Nam Hoang Dang, Houston, TX (US); Chikao Morimoto, Tokyo (JP)

Correspondence Address: FULBRIGHT & JAWORSKI L.L.P., 600 CONGRESS AVE., SUITE 2400, AUSTIN, TX, 78701, US

(21) Appl. No.: 10/514,738

(22) PCT Filed: May 15, 2003
(86) PCT No.: PCT/US03/15499

§ 371 Date: Oct. 19, 2005

(30) Foreign Application Priority Data
May 17, 2002 (US) 60381606

(51) Int. Cl. A61K 51/00; A61K 48/00; A61K 39/395; A61K 38/17; A61K 31/704; A61K 31/7048; A61K 31/4745

(52) U.S. Cl. 424/1.49; 514/12; 514/27; 514/34; 514/285; 424/450; 424/178.1; 424/93.2

(57) ABSTRACT

Therapeutic methods comprising administering a composition comprising CD26 thereof in conjunction with chemotherapeutic agents or radiotherapeutic agents for the prevention and treatment of cancers are provided. Also provided are methods for potentiating immune responses comprising administering a composition comprising CD26.

The present application claims priority to U.S. Provisional Application Ser. No. 60/381,606, filed May 17, 2002. The entire contents of the above-referenced application in incorporated herein by reference and without disclaimer. The government owns rights in the present invention pursuant to grant number AR33713 from the National Institutes of Health.

### BACKGROUND OF THE INVENTION

1. Field of the Invention

The present invention relates generally to the fields of cancer and immunology. More particularly, it concerns the therapeutic use of CD26 in the treatment of cancers, including gene-based and/or protein-based therapies of CD26, in combination with chemotherapeutic agents. Methods for potentiating immune responses to infections and methods for treating and preventing immunosuppression using CD26-based therapies are also described.

2. Description of Related Art

Cancer has become one of the leading causes of death in the western world, second only behind heart disease. Current estimates project that one person in three in the U.S. will develop cancer, and that one person in five will die from cancer. Currently, there are few effective options for the treatment of cancer. Radiation therapy and chemotherapy are generally used in the treatment of cancer. However, cancer cells often become resistant to both radiation and chemotherapy. Furthermore, chemotherapy is typically associated with numerous side-effects.

A molecule called CD26, which is known to be involved in various aspects of immune regulation, is also known to be associated with the development of certain human tumors. Among its varied functions, CD26 also acts as an extracelluar peptidase and is known as dipeptidyl peptidase IV (DPPIV), due to its enzymatic activity. CD26 is known to be associated with certain types of cancers. For example, cancers that are DPPIV-positive or express CD26 include most lung adenocarcinomas (Asada et at., 1993), differentiated thyroid carcinomas (Tanaka et al., 1995), B-chronic lymphocytic leukemia cells (Bauvois et al., 1999), T-cell lymphoblastic lymphomas/acute lymphoblastic leukemias (LBL/ALL) and T-cell CD30+ anaplastic large cell lymphomas (Carbone et al., 1995; Carbone et al., 1994).

CD26 also appears to have a role in melanoma development as its expression is lost with malignant transformation of melanocytes (Morrison el al., 1993; Wesley et al., 1999). G1 arrest following enforced CD26 expression has been observed in melanoma cells (Wesley et al., 1999). However, while CD26 has been used as a diagnostic tool to analyze the nature of a particular cancer, the art has not conducted investigations on its mechanisms of action in cancers.

### SUMMARY OF THE INVENTION

Some of the current goals of cancer research are to find methods that enhance the effects of the available chemotherapeutic agents using minimal doses to reduce the associated side-effects. Methods that make cancer cells more sensitive to chemotherapeutic/radiotherapeutic agents as well as methods that allow chemotherapeutic agents to act more selectively on cancer-cells rather than on normal cells are desired.

One aspect of the present invention addresses these goals by demonstrating that expression of CD26 peptides or proteins enhances the susceptibility of cancer cells to chemotherapeutic agents and/or radiotherapeutic agents.

Therefore, the invention provides methods-for inhibiting the growth of a cell comprising contacting the cell with: a) a CD26 composition; and b) a chemotherapeutic agent and/or a radiotherapeutic agent, in dosages effective to inhibit growth of the cell. In certain embodiments of these methods, the CD26 composition exhibits the dipeptidyl peptidase IV (DPPIV) activity.

In some embodiments, the cell is simultaneously contacted with the CD26 composition and the chemotherapeutic agent and/or the radiotherapeutic agent. In other embodiments, the cell is contacted with the CD26 composition prior to being contacted with the chemotherapeutic agent and/or the radiotherapeutic agent. In yet other embodiments, the cell is contacted with the CD26 composition after being contacted with the chemotherapeutic agent and/or radiotherapeutic agent.

Any chemotherapeutic agent that is effective for cancer therapy may be used in the practice of the present invention. Some non-limiting examples include mitomycin, actinomycin D, bleomycin, plicomycin, taxol, vincristine, vinblastine, carmustine, melphalan, cyclophosphamide, chlorambiicil, busulfan, lomustine, visplatin, tumor necrosis factor, cisplatin (CDDP), carboplatin, procarbazine, mechlorethamine, camptothecin, ifosfamide, nitrosurea, tamoxifen, raloxifene, estrogen receptor binding agents, gemcitabien, navelbine, fainesyl-protein transferase inhibitors, transplatinum, 5-fluorouracil, methotrexate, temazolomide (an aqueous form of DTIC), camptothecin, mitomycin C, adriamycin, doxorubicin, verapamil, etoposide, podophyllotoxin and/or any analog and/or derivative and/or variant and/or liposomal formulation of the foregoing.

In some embodiments, the chemotherapeutic agent is a DNA damaging agent. In other specific embodiments, the chemotherapeutic agent is an agent that cross-links DNA, an agent that alkylates DNA, an agent that intercalates DNA, an agent that leads to chrornosomal and mitotic aberrations by affecting nucleic acid synthesis, an agent that affects DNA replication, mitosis, or chromosomal segregation or a topoisomerase inhibitor.

In some embodiments, the chemotherapeutic agent is a topoisomerase II inhibitor and may be an anthracycline antibiotic, an amsacrine, an ellipticine, an epipodophyllotoxin, a mitoxantrone, a synthetic inhibitor or a derivative thereof.

Certain specific examples of topoisomerase II inhibitors include doxorubicin, etoposide, daunorubicin, teniposide, mitoxantrone, and derivatives and liposomal formulations thereof. In addition, synthetic topoisomerase II inhibitor such as small molecules are also contemplated.

Radiotherapeutic agents that may be used include γ-irradiation, X-rays, UV-irradiation, microwaves, electronic emissions, radioisotopes and the like.

In some embodiments, the CD26 composition is attached to a targeting agent. In such embodiments, a targeting agent capable of targeting the CD26 composition to a specific cancer cell type may be conjugated with a CD26 composition to target the CD26 composition to the cancer cell. Exemplary agents include, but not are limited to, antibodies specific for a tumor cell marker, growth factors, chemokines, cytokines, toxins, or other ligands/molecules that recognize specific molecules on the target cells. Non-limiting examples of tumor markers known in the art include carcinoembryonic antigen, prostate specific antigen, urinary tumor associated antigen, fetal antigen, tyrosinase (p 97), gp 68, TAG-72, HMFG, Sialyl Lewis Antigen, MucA, MucB, PLAP, estrogen receptor, laminin receptor, erb B, neu, and p 155 and antibodies to such tumor markers are contemplated as targeting agents to target CD26compositions to cancer cells expressing such tumor markers. Non-limiting examples of ligands are those that bind to a receptor that is expressed differentially on cancer cells; such as epidermal growth factor etc.

In yet other aspects, an immunotherapeutic antibody or a targeting antibody may be conjugated to a CD26 composition peptide/protein or expression vector encoding CD26), and be further conjugated to a radiotherapeutic agent, a toxin or another anticancer agent.

In some embodiments, the CD26 composition comprises an expression construct comprising a DNA segment that encodes SEQ ID NO. 1; SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 36, or a fragment, isoform, mutation, or variant thereof under the control of a promoter active in the cell. In some specific embodiments, the CD26 expression construct comprises a nucleic acid that encodes for at least the amino acid sequence Gly-Trp-Ser-Tyr-Gly (SEQ ID: NO: 42). In yet other specific aspects, the CD26 expression construct comprises a nucleic acid that encodes amino acids that exhibit the DPPIV enzymatic activity.

In some embodiments, the promoter of the expression construct is a heterologous promoter. The promoter may be a constitutive promoter, a tissue-specific promoter, an inducible promoter, or a noninducible promoter. Cancer cell specific-promoters are also contemplated.

In other embodiments, the expression construct is a viral expression construct and may be a retroviral construct, an adenoviral construct, an adeno-associated viral construct, a herpesviral construct, a polyoma viral construct, a vaccinia viral construct or a lentiviral construct.

Alternatively, the expression construct may be a non-viral expression construct. Non-viral expression constructs may be administered as a naked DNA or in a liposomal formulation.

In other embodiments, the CD26 composition is a CD26 peptide or protein that comprises an amino acid sequence of SEQ ID NO 2, SEQ ID NO: 4, SEQ ID NO: 33, SEQ ID NO: 35, SEQ ID NO: 37, or a fragment, mutation, isoform or biologically functionally equivalent thereof. In some specific aspects, the CD26 peptide or protein comprises an amino acid sequence that encodes at least amino acids 628-632 of SEQ ID NO 2.

In yet other-embodiments, the CD26 peptide or protein may be a soluble CD26 protein or peptide, a recombinantly produced CD26 peptide or protein, a CD26 fusion peptide or protein, a substantially purified CD26 peptide or protein, a partially purified CD26 peptide or protein, a naturally occurring CD26 peptide or protein, an isoform of a naturally occurring CD26 peptide or protein, or a mutant CD26 peptide or protein.

In yet other embodiments of the methods, the cell is a cancer cell and may be any cancer cell including a hematological cancer cell, a bladder cancer cell, a blood cancer cell a breast cancer cell, a lung cancer cell, a colon cancer cell, a prostate cancer cell, a liver cancer cell, a pancreatic cancer cell, a stomach cancer cell, a testicular cancer cell, a brain cancer cell, a thyroid cancer cell, an ovarian cancer cell, a lymphatic cancer cell, a skin cancer cell, a brain cancer cell, a bone cancer cell, a soft tissue cancer cell.

In other embodiments, the cell is located in a human subject. In such embodiments the CD26 composition may be administered systemically. Administration by intravenous, intraarterial, intraperitoneal, intradermal, intratumoral, intramuscular, subcutaneous, intraarthricular, intrathecal, oral, dermal, nasal, buccal, rectal, or vaginal routes is contemplated. In yet other embodiments, the CD26 composition is administered locally to a tumor and this may be via direct intratumoral injection or by injection into tumor vasculature. All these methods of administration are known to the skilled artisan.

The invention also provides methods of inducing cell-cycle arrest in a cell comprising contacting the cell with: a) a CD26 composition; and b) a chemotherapeutic agent and/or a radiotherapeutic agent, in dosages effective to induce cell-cycle arrest in the cell. In such methods, the cell may be a cancer cell.

The invention also provides methods of killing a cancer-cell comprising contacting the cell with: a) a CD26 composition; and b) a chemotherapeutic agent and/or a radid therapeutic agent, in dosages effective to kill said cancer cell.

Further provided are methods of potentiating the effect of a chemotherapeutic agent and/or a radiotherapeutic agent on a tumor cell comprising contacting said tumor cell with a CD26 composition and the DNA damaging agent.

Thus, the invention provides method of treating cancer in a human patient comprising administering: a) a CD26 composition; and b) a chemotherapeutic agent and/or a radiotherapeutic agent, to the human patient, wherein the dose of the CD26 composition, when combined with the dose of the chemotherapeutic and/or radiotherapeutic agent, is effective to treat the cancer.

In certain embodiments of these methods, the CD26 composition is a nucleic acid encoding a CD26 peptide or protein and is a viral vector. In such embodiments, the viral vector dose is from about 10³, 10⁴, 10⁵, 10⁶, 10⁷, 10⁸, 10⁹, 10¹⁰, 10¹¹, 10¹², 10¹³ pfu and higher. Alternatively, dosage may be expressed in units of viral particles (vp); thus, the numbers listed above in “pfu” units may be expressed in units of “vp” units or “viral particles.” It is contemplated that about 10³ to about 10¹⁵, about: 10⁵ to about 10¹², or 10⁷ to about 10¹⁰ viral particles may be administered to a patient.

The methods of the present invention include dispersing expression constructs, vectors, and cassettes in pharmacologically acceptable solution for administration to a patient. The pharmacologically acceptable solution can be a buffer, a solvent a diluent and may comprise a lipid. In one embodiment of the present invention, a nucleic acid molecule encoding a CD26 polypeptide is administered in a liposome. These nucleic acid molecules may be administered to the patient intravenously, intraperitoneally, intratracheally, intratumorally, intramuscularly, endoscopically, intralesionally, percutaneously, subcutaneously, regionally, or by direct injection or perfusion. It is further contemplated that treatment methods may involve multiple administrations. The nucleic acid of the present invention may be administered by injection. Other embodiments include the administering of the nucleic acid by multiple injections.

In other embodiments, it is contemplated that protein/peptide compositions of CD26 including soluble forms, partially purified, substantially purified, recombinantly produced, and artificially synthesized forms will be administered to the patients. Liposomal formulations of such peptides or proteins are also contemplated.

In some aspects, the chemotherapeutic agent is a topoisomerase II inhibitor. The methods of the invention further comprise treating the patient with another anticancer agent, wherein the other anticancer agent is a therapeutic polypeptide, a nucleic acid encoding a therapeutic polypeptide, an immunotherapeutic agent, a cytokine, a chemokine, an activating agent, or a biological response modifier. The other anticancer agent may be administered simultaneously with the CD26 composition and DNA damaging agent. Alternatively, the other anticancer agent may be administered at a different time than the CD26 composition and DNA damaging agent.

The invention also provides methods of inducing tumor regression comprising administering to a patient in need thereof: a) a CD26 composition; and b) a chemotherapeutic agent and/or a radiotherapeutic agent, in dosages effective to cause regression of said tumor.

The invention also provides methods of inducing tumor necrosis comprising administering to a patient in need thereof: a) a CD26 composition; and b) a chemotherapeutic agent and/or a radiotherapeutic agent, in doses effective to induce tumor necrosis.

Furthermore, the invention provides method of treating a patient having a cancer comprising, induction of CD26 expression in cells of said cancer, and administering a chemotherapeutic and/or a radiotherapeutic agent to said patient whereby the expression of CD26 enhances the sensitivity of the cancer cell to the chemotherapeutic and/or radiotherapeutic agent. In some non-limiting examples, the chemotherapeutic is a topoisomerase II inhibitor. In some embodiments, the induction of CD26 expression in cells of said cancer is by contacting the cells with a biological factor. Examples of biological factors that can induce CD26 expression are cytokines, chemokines, retinoids, interferons, chemotherapeutic agents, antibodies, or antigenic molecules. Alternatively, the induction of CD26 expression in a cancer cells may be achieved by contacting the cell with a chemical agent.

The present invention also provides methods for increasing topoisomerase II expression in a cell comprising contacting the cell with a CD26 composition. In some specific embodiments, the topoisomerase II is topoisomerase II α.

Also provided are methods for increasing the sensitivity to apoptosis by contacting the cell with a CD26 composition. In some embodiments of this aspect, the sensitivity to apopotosis is enhanced by the increase in topoisomerase II expression caused by the CD26 composition in the cell. In yet other specific embodiments, the topoisomerase II is topoisomerase II α. In other embodiments, the invention provides methods for inducing apopotosis comprising adminsitration of a CD26 composition to a cell.

The present invention has shown that CD26 directly influences antigen presenting cells (APC), which once activated, present antigens to T-cells and cause activation of T-cells followed by proliferation of activated T-cells. Both cytotoxic T lymphocytes (CTLS) and T-helper cells are activated by APC's causing an upregulation of the immune system. CD26 compositions, including expression vectors encoding CD26, soluble CD26 proteins as well as other protein/peptide compositions described herein, activate APC's and thereby potentiate immune responses.

Hence, the invention also provides methods for activating antigen presenting cells (APCs), comprising providing to the cells a CD26 composition. Any type of a antigen presenting cell may be used, such as a dendritic cell, a macrophage, an endothelial cell, glia, and the like.

In some embodiments, the CD26 composition is further attached to a targeting agent capable of targeting the CD26 composition to a antigen presenting cell. Exemplary agents include, but not are limited to, antibodies or ligands specific for APC cell surface specific proteins. Methods for delivery of CD26 compositions are described in the specification.

In some embodiments, the methods further comprise providing to the antigen presenting cells an antigen. The antigen may be provided in the form of a nucleic acid expression vector, including viral and non-viral vectors, that encodes the antigen. Alternatively, the antigenic protein or peptide may be provided to the cell. If expression vectors are used, they will be under the control of suitable promoters. The specification provides a detailed description of expression vectors and promoters. In some embodiments, the promoter may be APC cell specific promoters. The use of adjuvants and other agents generally used in the art to boost immune responses are also contemplated. Various types of antigens may be provides and these include tumor antigens, bacterial antigens, viral antigens, and fungal antigens. Several methods to specifically deliver viral vectors to antigen presenting cells are known in the art. For examples, U.S. Pat. No. 6,300,090, teaches the use of viral vectors to deliver antigens to dendritic cells for processing and presentation to T cells.

Antigen presenting cells, that express CD26 and optionally one or more antigens, stimulate T helper cells as well as CTL's, both events lead to potentiation of the immune response. As is well known in the art, T-helper cell activation leads to a wide range of immune regulatory activities including the release of cytolines, interferons, cell-cell interactions, activation of B-cells and even the activation of CTL's.

In one aspect, the antigen-presenting cells that express CD26 and optionally one or more antigens can be used to stimulate cytotoxic T cell lymphocyte (CTL) proliferation both ex vivo or in vivo. The ex vivo expanded CTL can be administered to a human patient in a method of adoptive immunotherapy.

Additionally the invention provides methods for potentiating immune responses of an animal comprising activating the antigen presenting cells of the animal by administering a CD26 composition to the animal. In some aspects, these methods may further comprise providing to the antigen presenting cells of the animal an antigen such as a tumor antigen, a bacterial antigen, a viral antigen, and/or a fungal antigen. In some embodiments, the animal may be a human. The human may be immunosuppressed, and/or afflicted with cancer and/or afflicted with an infection caused by a viral, bacterial, or fungal pathogen. It is contemplated that the methods for potentiating immune responses may be used with other therapies generally used to treat such. conditions including other antibiotics, antiviral agents, anti-tumor agents and therapies. One of skill in the art will recognize this and other scenarios for potentiating immune responses using methods of the invention.

As used herein the specification, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Effect of CD26/DPPIV expression on doxorubicin-mediated growth inhibition. CD26 Jurkat transfectants were incubated at 37° C. in culture media alone or culture media containing doxorubicin at the concentrations indicated, and an MTT uptake assay was performed as described in “Examples.” wtCD26, wild-type CD26 Jurkat transfectant; S630A, CD26-positive/DPPIV-negative mutant CD26 Jurkat transfectant; control, nontransfected Jurkat; 340-4, CD26-positive/DPPIV-positive mutant CD26 Jurkat transfectant; neo, plasmid-only Jurkat transfectant. Data represent the means of three separate experiments. Cytotoxicity index was calculated as follows:

$$cytoxicity index ( \% of control ) = 1 - \frac{{A}_{570 / nm} of treated cells}{{A}_{570 / nm} of control cells} \times 100 \%$$

FIG. 2. Effect of CD26/DPPIV expression on doxorubicin-mediated cell cycle arrest at G₂-M. CD26 Jurkat transfectants were incubated at 37° C. in media containing doxorubicin for 24 h. Cells were then harvested, and cell cycle analyses were performed as described in “Examples.” Data are representative of three separate experiments. Numerical data are shown in Table 5.

FIGS. 3A, 3B, & 3C. Effect of exogenous sDPPIV on doxorubicin-mediated growth inhibition of Jurkat cells. Jurkat cells were incubated at 37° C. in culture media alone, culture media with doxorubicin alone at the indicated concentrations, culture media with sDPPIV (50 μg/ml) alone, and culture media with doxorubicin at the indicated concentrations and sDPPIV (50 μg/ml). MTT assays were performed. Data represent the means of three separate experiments. (FIG. 3A), control; (FIG. 3B), wtCD26; (FIG. 3C), S630A. Cytotoxicity index was calculated as follows:

$$cytoxicity index ( \% of control ) = 1 - \frac{{A}_{570 / nm} of treated cells}{{A}_{570 / nm} of control cells} \times 100 \%$$

FIGS. 4A & 4B. Effect of exogenous sDPPIV on doxorubicin-mediated growth inhibition of B-lymphoid cell lines. Cells were incubated at 37° C. in culture media alone, culture media with doxorubicin alone at the indicated concentrations, culture media with sDPPIV (50 μg/ml) alone, and culture media with doxorubicin at the indicated concentrations and sDPPIV (50 μg/ml). MTT assays were performed. Data represent the means of three separate experiments (FIG. 4A), Jiyoye; (FIG. 4B), Namalwa. Cytotoxicity index was calculated as follows:

$$cytoxicity index ( \% of control ) = 1 - \frac{{A}_{570 / nm} of treated cells}{{A}_{570 / nm} of control cells} \times 100 \%$$

FIG. 5. Effect of CD26/DPPIV expression on etoposide-mediated growth inhibition. CD26 Jurkat transfectants and parental cells were incubated at 37° C. in culture media alone or culture media containing etoposide at the concentrations indicated, and MTT uptake assay was performed. wtCD26: wild type CD26 Jurkat transfectant; S630A: CD26-positive/DPPIV-negative mutant CD26 Jurkat transfectant. Data represent the means of three separate experiments. Cytotoxicity index was calculated as follows:

$$cytoxicity index ( \% of control ) = 1 - \frac{{A}_{570 / nm} of treated cells}{{A}_{570 / nm} of control cells} \times 100 \%$$

FIG. 6. CD26 expression and DPPIV enzyme activity on Jurkat transfectants. Jurkat cells were evaluated for CD26 expression by flow cytometry as described in “Examples.” Panel 1: Parental Jurkat; Panel 2: S630A transfectants; Panel 3: wtCD26 transfectants; a: negative control; b: anti-CD26 antibody.

FIGS. 7A & 7B. Enhanced sensitivity of wtCD26 to etoposide and doxorubicin in serum-free media. Following pre-treatment with AIM V serum-free media at 37° C. for 24 hours, wtCD26 and parental Jurkat cells were incubated at 37° C. in serum-free media containing etoposide (FIG. 7A) or doxorubicin (FIG. 7B) for 48 hours, and MTT uptake assays were performed. Data represent the means of three separate experiments. Cytotoxicity index was calculated as follows:

$$cytoxicity index ( \% of control ) = 1 - \frac{{A}_{570 / nm} of treated cells}{{A}_{570 / nm} of control cells} \times 100 \%$$

FIG. 8. Proliferative effect on PBMC by sCD26. PBMC (1×10⁵) were incubated in culture medium with or without TT or sCD26. The freshly isolated PBMC was cultured in standard culture medium for 24 h, then TT (0.5 μg/ml) was added to the medium to incubate for an additional 16 h. sCD26 (0.5 μg/ml) was then added to the wells and was incubated for various time periods as shown. Proliferation of cells was monitored in all instance by measuring [³H]TDR incorporation on day 7 of culture after the first sCD26 was added. Degree of proliferation is indicated as cpm in the ordinate. The experiments represent mean values±SE calculated from three independently performed experiments.

FIG. 9. PBMC subpopulation that takes up sCD26. Freshly isolated PBMC (1×10⁶/well) were incubated with or without TT for 16 h after a 24-h incubation in standard medium alone, and then sCD26-OG was added to the culture wells. After incubation with sCD26-OG for 24 h, cells were washed in PBS and in acidic buffer (pH 3 PBS) to strip any sCD26-OG on the cell surfaces. The cells were then incubated with the various PE-conjugated Abs, and the intensity of sCD26-OG was measured for flow histograms. Analysis was performed by FACSCalibur. Similar experiments were performed with mutant sCD26 of defective DPPIV activity.

FIGS. 10A & 10B. Reconstitution study to determine the target cells of sCD26. (FIG. 10A) Freshly isolated monocytes (0.5×10⁶/well) were preincubated with 0.5 μg/ml TT and with different concentrations of sCD26 for 24 h, then, after washing with PBS, 1.0×10⁴/well of the preincubated monocytes were subjected to the assay with 1×10⁵/well of purified T cells. (FIG. 10B), A total of 1×10⁵/well of purified T cells were preincubated with 0.5 μg/ml of TT and with different concentrations of sCD26 for 24 h. Cells were then washed with PBS and subjected to the assay. Proliferation of cells was monitored in all instances by measuring [³H]TdR incorporation on day 7 of culture. Degree of proliferation is indicated as cpm in the ordinate. The experiments represent mean values±SE calculated from three independently performed experiments.

FIGS. 11A & 11B. M6P/IGF-IIR plays a role in incorporating sCD26 into monocytes. The freshly isolated monocytes (0.5×10⁶/well) were incubated with (FIG. 11A) or without (FIG. 11B) TT for 16 h after a 24-h incubation in standard medium alone, and then sCD26 was added to the culture wells in the presence of various concentration of M6P (0, 0.1, 1.0, and 10.0 μM/well). After a 24-h incubation, cells were washed in ice-cold PBS and in acidic PBS to strip any sCD26-OG on the cell surface. Analysis was performed by FACSCalibur. The intensity of sCD26-OG is shown in flow histograms. Similar experiments using mutant sCD26/DPPIV⁻ were performed. The data in this study represent one of three independently performed experiments. The abscissa represents fluorescence intensity (log₁₀ scale), and the ordinate represents the respective cell number.

FIGS. 12A & 12B. Enhancing effect of TT-induced T cell proliferation by sCD26 did not result from trimming of the MHC-bound peptide on monocytes. Freshly isolated monocytes (0.5×10⁶/well) were preincubated with 0.5 μg/ml TT for 16 h, followed by a 24-h incubation with sCD26 at different doses before (FIG. 12A) or after (FIG. 12B) being treated with 0.05% glutaraldehyde for 30 s at room temperature. After washing with PBS, 1×10⁴/well of the preincubated monocytes were then subjected to the assay with 1×10⁵/well of purified T cells. Similar experiments were performed with mutant sCD26/DPPIV⁻. Proliferation of cells was monitored in all instances by measuring [³H]TdR incorporation on day 7 of culture. Degree of proliferation is indicated as cpm in the ordinate. The experiments represent mean values±SE calculated from three independently performed experiments.

FIG. 13. sCD26/DPPIV induces up-regulation of the costimulatory molecule CD86 on monocytes. Freshly isolated monocytes (0.5×10⁶/well) were incubated with or without TT for 16 h after a 24-h incubation in the standard medium alone, and then sCD26 (0.5 μg/ml) was added to the culture wells. After incubation with sCD26 for different hours, cells were washed with PBS, incubated with FITC-conjugated CD80, CD86, or HLA-DR, Abs, and then analysis was performed by FACSCalibur. The histogram shown in this figure is the increased intensity of CD86-FITC on monocytes cultured for 24 h with sCD26/PPIV⁺ after TT treatment. The single histogram profile of the CD86 expression shown in this figure is one of three representative experiments. The abscissa represents fluorescence intensity (log₁₀ scale), and the ordinate represents the respective cell number.

FIGS. 14A & 14B. Inhibitory effect of CD86 mAb and CTLA-4 Ig on T cell proliferation induced by TT/sCD26-treated monocytes. (FIG. 14A), Freshly isolated monocytes (0.5×10⁶/well) were incubated with or without TT for 16 h after a 24-h incubation in the standard medium alone, and then sCD26 (0.5 μg/ml) was added to the culture wells (the presence or absence of TT/sCD26 is indicated in the box to the right). After incubation with or without sCD26 for 24 h, monocytes were incubated with the indicated. mAbs (5 μg/ml) or CTLA-4 Ig (5 μg/ml) for 15 min at 4° C. before onset of culture. Inhibition on T cell proliferation was expressed as the percentage of reactivity of control cultures without addition of mAbs or control human Ig performed in parallel. (FIG. 14B), Dose-dependent inhibitory effects of anti-CD86 mAb and human CTLA-4 Ig (0.1-20 μg/ml) on T-cell proliferative response induced by TT/sCD26-treated monocytes. The results were expressed as the percentage of the mean values obtained in the presence of the isotype-matched negative control or control murine Ig. Proliferation of T cells was monitored in all instances by measuring [³H]TdR incorporation on day 7 of culture. Bars are representative of mean values of percentage of inhibition or proliferation±SE of three independently performed experiments.

FIGS. 15A & 15B. Enhancing effect of CD26/DPPIV surface expression on apoptosis induced by topoisomerase II inhibitors. CD26 Jurkat transfectants were incubated at 37° C. in culture media alone or culture media containing etoposide (FIG. 15A) for 14 hours or doxorubicin (FIG. 15B) for 16 hours at the concentrations indicated. Cells were then harvested and Annexin V/PI assays were performed as described in Example 3. wtCD26: wild-type CD26 Jurkat transfectant; S630A: Jurkat cells transfected with mutant CD26 containing an alanine at the putative catalytic serine residue at position 630, resulting in a mutant CD26-positive/DPPIV-negative Jurkat transfectant; control: nontransfected parental Jurkat; 340-4: Jurkat cells transfected with mutant CD26 containing point mutations at the ADA-binding site residues 340-343,with amino acids L₃₄₀, V₃₄₁, A₃₄₂, and R₃₄₃ being replaced by amino acids P₃₄₀, S₃₄₁, E₃₄₂, and Q₃₄₃, resulting in a mutant CD26-positive/DPPIV-positive mutant CD26 Jurkat transfectant incapable of binding ADA. Data are representative of three separate experiments.

FIG. 16. CD26/DPPIV-associated enhancement in PARP cleavage induced by topoisomerase II inhibitors. CD26 Jurkat transfectants were incubated at 37° C. with media containing etoposide for 16 hours or doxorubicin for 18 hours at the indicated doses. Cells were then harvested, and whole cell lysates were obtained. Following SDS-PAGE of lysates, immunoblotting studies for PARP and β-actin were performed as described in Example 3. The cleaved product of PARP was detected at ˜85 kDa Each lane was loaded with 30 μg of protein.

FIG. 17. Time course study of the effect of CD26/DPPIV surface expression on etoposide-induced apoptosis. Jurkat cells were incubated at 37° C. with media containing etoposide (3 μM) for the indicated time periods at the indicated doses. Cells were then harvested, and cytosol fractions were obtained as described in Example 3. Following SDS-PAGE of lysates, immunoblotting studies with specific antibodies for PARP, caspase-9, caspase-3, Apaf-1, Bcl-xl, and β-actin were performed as described in Example 3 (*): caspase-3 cleaved products; (**): Bcl-xl cleaved products. Each lane was loaded with 30 μg of protein.

FIG. 18. Effect of caspase-9 inhibitor z-LEHD-fmk on etoposide-induced apoptosis in wtCD26 Jurkat transfectant. wtCD26 Jurkat transfectants were incubated at 37° C. for 2 hours of preincubation with z-LEHD-fmk at varying doses, and then treated with 3 μM etoposide for 16 hours. Cells were then harvested, and whole cell lysates were obtained as described in Example 3. Following SDS-PAGE of lysates, immunoblotting studies for PARP, caspase-3, caspase-9, and β-actin were performed as described in Example 3. (*): caspase-3 cleaved products. Each lane was loaded with 30 μg of protein.

FIGS. 19A, 19B, 19C, 19D & 19E. Effect of inhibition of DPPIV activity on topoisomerase II alpha expression. (FIG. 19A) After incubation of Jurkat cells at 37° C. for 24 hours in culture media, cells were harvested, and nuclear extracts were obtained. Following SDS-PAGE of lysates, immunoblotting studies were performed for topoisomerase II alpha or β-actin as described in Example 3. Each lane was loaded with 30 μg of protein. Lane 1: wtCD26 Jurkat transfectant, lane 2: S630A mutant transfectant, lane 3: parental Jurkat. (FIG. 19B) wtCD26 Jurkat transfectants or parental Jurkat were incubated in culture media alone (DFP−), culture media containing 100 μM DFP for 2 hours or for 6 hours (DFP+). A representative sample of cells reflecting each treatment condition was obtained, and DPPIV enzyme activity assays were then performed as described in Example 3. (FIG. 19C) wtCD26 Jurkat transfectants (lanes 1-3) or parental Jurkat (lanes 4-6) were incubated in culture media alone (lanes 1, 3), culture media containing 100 μM DFP for 2 hours (lanes 2, 5) or for 6 hours (lanes 3, 6). Cells were harvested, and nuclear extracts were obtained. Following SDS-PAGE of lysates, inmmunoblotting studies for topoisomerase II alpha or β-actin were performed as described in Materials and Methods. Each lane was loaded with 30 μg of protein. (FIG. 19D) wtCD26 Jurkat transfectants were incubated in culture media (bar I), or in-culture media with 100 μM DFP for 4 hours (bar II), or they were incubated in culture media with 100 μM DFP for 4 hours, then washed twice in PBS to ensure removal of DFP followed by incubation in culture media for 2 hours (bar III) or 8 hours (bar IV). A representative sample of cells reflecting each treatment condition was obtained, and DPPIV enzyme activity assays were then performed. (FIG. 19E) wtCD26 Jurkat transfectants were incubated in culture media (lane 1), or in culture media with 100 μM DFP for 4 hours (lane 2), or they were incubated in culture media with 100 μM DFP for 4 hours, then washed twice in PBS to ensure removal of DFP followed by incubation in culture media for 2 hours (lane 3) or 8 hours (lane 4). Cells were then harvested, and nuclear extracts were obtained. Following SDS-PAGE of lysates, immunoblotting studies for topoisomerase II alpha or β-actin were performed. Each lane was loaded with 30 μg of protein.

FIG. 20. Effect of soluble CD26 molecules on topoisomerase II alpha expression. Parental Jurkat cells were incubated overnight in culture media alone (−) or culture media containing soluble CD26 (sCD26) molecules (300 μg/ml) (+) at 37° C. Cells were then harvested, and nuclear extracts were obtained. Following SDS-PAGE of lysates, immunoblotting studies for topoisomerase II alpha or β-actin were performed. Each lane was loaded with 30 μg of protein.

FIG. 21. sCD26-associated enhancement of doxorubicin-induced PARP cleavage. Parental Jurkat cells were incubated overnight in culture media alone (−) or culture media containing soluble CD26 (sCD26) molecules (300 μg/ml) (+) at 37° C., followed by incubation with doxorubicin at the indicated concentrations for 16 hours. Cells were then harvested, and whole cell lysates were obtained. Following SDS-PAGE of lysates, immunoblotting studies for PARP or β-actin were performed. Each lane was loaded with 30 μg of protein.

FIGS. 22A, 22B. Effect of CD26/DPPIV on DR5 expression induced by etoposide treatment. (FIG. 22A) Jurkat cells were incubated at 37° C. in culture media containing etoposide (3 μM) for the indicated time periods at the indicated doses. Cells were then harvested, and whole cell lysates were obtained. Following SDS-PAGE of lysates, immunoblotting studies for DR5 and β-actin were performed. Each lane was loaded with 30 μg of protein. Anti-DR5 mAb detects two bands of 58 kDa and 32 kDa. (FIG. 22B) Following 2 hours of pre-incubation at 37° C. with varying doses of z-LEHD-fink, wtCD26 Jurkat transfectants were treated with 3 μM etoposide for 48 hours. Cells were then harvested, and whole cell lysates were obtained. Following SDS-PAGE of lysates, immunoblotting studies for DR5, caspase-9, and β-actin were performed. Each lane was loaded with 30 μg of protein.

### DESCRIPTION OF ILLUSTRATIVE EMBODIMENTS

The field of cancer therapy is hindered by the fact that not all types of cancer cells are susceptible to chemotherapeutic agents or radiotherapeutic agents. Furthermore, even when one or more chemotherapeutic/radiotherapeutic agents are effective against a particular cancer, they cause several side effects. As a result, there is an acute need for methods that increase the susceptibility of cancer cells to chemotherapeutic/radiotherapeutic agents and/or methods that allow the effective use of lower doses of chemotherapeutic/radiotherapeutic agents.

The present invention provides therapeutic methods that deliver CD26 peptides or proteins or increase the expression of CD26 peptides or proteins in cells, which in turn increase the susceptibility of the cell to chemotherapeutic DNA damaging agents and/or radiotherapeutic agents. Methods for selectively expressing the CD26 peptides or proteins in cancer cells versus normal cells are also provided. The present inventors have shown that the expression of CD26 in cancer cells, followed by treatment with chemotherapeutic agents disrupts cell cycle events, induces cell-cycle arrest, and causes growth inhibition of the cancer cells. Therefore, the methods of the present invention significantly increase the efficacy of existing methods for the treatment of cancers.

One effect of the expression of CD26 and/or its enzymatic DPPIV activity is an increase in the expression of topoisomerase II which in turn increases the sensitivity of the cell to chemotherapeutic agents that inhibit or poison topoisomerase II. Topoisomerase inhibitors are well known in the art and are widely used to treat cancers. Other effects of CD26 on enhancing sensitivity of cancer cells to chemotherapeutic agents are also contemplated. Although, the CD26 protein is known to be involved in a variety of functional aspects, the increase in topoisomerase II expression in cells following the expression of CD26, is the first demonstration of a functional association between CD26 and topoisomerase.

The present inventors have also demonstrated that expression of CD26 or the presence of a CD26protein in a cell increases the sensitivity of a cell to apoptosis. In some embodiments, this enhanced sensitivity to apopotosis is due to the the increase in topoisomerase II expression caused by the CD26 protein. Thus, the invention also provides methods for inducing apoptosis as well as methods for enhancing apoptosis in a cancer cell following either a) inducing the expression of CD26, and/or b) expressing recombinant CD26, and/or 3) providing to the cell a CD26 protein composition.

The present inventors have also demonstrated that CD26 enhances T-cell immune responses to antigens by directly affecting antigen presenting cells (APC's). Therefore, in addition to providing effective therapy against cancers, the present inventors contemplate therapeutic utility of CD26 for potentiating immune responses by activating APCs. Hence, the use of CD26 for the treatment of infections, tumors, and immunosuppressive conditions is also provided.

### A. CD26

CD26 is a 110-kd surface glycoprotein with an array of diverse functional properties that is expressed on a number of tissues, including epithelial cells and leukocyte subsets (Morimoto and Schlossman, 1998; von Bonin et al., 1998). The CD26 protein is a membrane-associated ectopeptidase that possesses dipeptidyl peptidase IV (DPPIV) activity in its extracellular domain and is able to cleave amino-terminal dipeptides from polypeptides with either L-proline or L-alanine at the penultimate position.

Work over the past decade has shown CD26 to be a molecule with a plethora of functions in basic human T-cell physiology. For example, CD26 cleaves certain chemokines involved in T-cell and monocyte function (Oravecz et al., 1997; Proost et al., 1998). Other studies have identified CD26 as the adenosine deaminase (ADA) binding protein which regulates ADA surface expression. It is believed that the CD26/ADA complex plays a key role in the catalytic removal of local adenosine to regulate immune system function (Dang et al., 1996; Kameoka et al., 1993; Morrison et al., 1993).

Although constitutively expressed in the liver, intestine and kidney, CD26 expression level is tightly regulated on T-cells, and its density is markedly enhanced after T-cell activation. In resting T-cells, CD26 is expressed on a subset of CD4+ memory T-cells, and this CD4+CD26 high T-cell population has been shown to respond maximally to recall antigens. In fact, CD26 itself is involved in the signal transducing process of T-cells under certain experimental conditions. Cross-linking of CD26 and CD3 with immobilized monoclonal antibodies (mAbs) can induce T-cell activation and IL-2 production. Anti-CD26 antibody treatment of T-cells results in an enhanced proliferative response to anti-CD3 or anti-CD2 stimulation. Moreover, while ligation of the CD26 molecule by anti-CD26 mAbs, such as 1F7, induces increased tyrosine phosphorylation of signaling molecules such as CD3zeta and p561ck, soluble anti-CD26 mAbs and other DPPIV inhibitors suppress T-cell growth and function in certain instances.

In addition, activation of T-cell by various stimuli increases CD26 surface expression and thus, CD26 is often used as a T-cell activation marker (Fox et al., 1984; Morimoto et al., 1989). CD26 is also a co-stimulatory surface molecule involved in the CD3 and CD2 pathways of T-cell activation.

While some reports show that the ability of CD26 to mediate activation signals is dependent on a functional CD3/TcR complex (von Bonin et al., 1998; Dang et al., 1990), the present inventors have show that CD26 can transmit signals resulting in alterations of T-cell biological responses in the absence of a functional CD3/TcR complex (Ho et al., 2001). In normal T-cells, engagement of CD26 results in an increased phosphorylation of proteins involved in T-cell signal transduction, mediated in part through the physical association of CD26 and CD45 (Hegen et al., 1997; Torimoto et al., 1991).

One aspect of the present invention demonstrates that CD26 enhances T-cell immune responses-to antigens by directly affecting antigen presenting cells (APC's). Previous studies by the inventors had demonstrated that soluble CD26 enhanced the proliferation of T-lymphocytes that is induced by the recall antigen tetanus toxoid (TT), (Tanaka et al., 1994). The inventors have now shown that CD26 upregulates the expression of the co-stimulatory molecule CD86 but not CD80 or HILA-DR Ag on monocytes. Thus, the-invention provides therapeutic uses of CD26 for potentiating immune responses, especially during infections and immunosuppressive conditions.

Besides its involvement in immunoregulation, CD26 may-have a role in the development of certain human tumors. Most lung adenocarcinomas are DPPIV-positive, while other histological types of lung carcinoma are DPPIV-negative (Asada et al., 1993). In addition, CD26 expression is high in differentiated thyroid carcinomas but is absent in benign thyroid diseases (Tanaka et al., 1995). High levels of CD26 protein expression and mRNA transcripts are found in B-chronic lymphocytic leukemia cells and activated B-cells, as compared to normal resting B-cells (Bauvois et al., 1999). Meanwhile, CD26 expression on T-cell malignancies appears to be restricted to aggressive pathologic entities such as T-cell lymphoblastic lymphomas/acute lymphoblastic leukemias (LBL/ALL) and T-cell CD30+ anaplastic large cell lymphomas, being detected only on a small percentage of indolent diseases such as mycosis fungoides. Significantly, within the T-cell LBL/ALL subset, CD26 expression is an independent marker of poor prognosis patients (Carbone et al., 1995; Carbone et al., 1994). CD26 also appears to have a role in development of melanoma as CD26 expression is lost with malignant transformation of melanocytes (Morrison et al., 1993; Wesley et al., 1999). Enforced CD26 expression was shown to induce G1 arrest in melanoma cells (Wesley et al., 1999). However, the present invention is the first to demonstrate that expression of CD26 increases the sensitivity of cancer cells to chemotherapeutic agents.

### B. TOPOISOMERASES AND TOPOISOMERASE INHIBITORS

The present invention demonstrates that one effect of expressing CD26 in a cell is an increase in the expression of topoisomerase II. Therefore, the expression of CD26 in a cell causes sensitivity of the cell to inhibitors of topoisomerase II.

Topoisomerases are a group of enzymes known to be important in DNA replication, DNA repair, genetic recombination and DNA transcription. They catalyze the introduction and relaxation of superhelicity in DNA. Several types of topoisomerases are known. For example, the topoisomerase I enzymes relax superhelical DNA, a process that is energetically spontaneous. The topoisomerase II enzymes, also known as the gyrases, catalyze the energy-requiring requiring and ATP-dependent introduction of negative superhelical twists into DNA. In DNA replication, topoisomerases I and II have the function of relaxing the positive superhelicity that is introduced ahead of the replicating forks by the action of helicases. In addition, gyrases introduce negative twists into segments of DNA that allow single-strand regions to appear. Thus, topoisomerization reactions of DNA such as supercoiling/relaxation, knotting/unknotting and catenation/decantenation are carried out by topoisomerases.

As a result of their role in replication of cells, DNA repair, recombination and transcription, topoisomerases have been implicated as targets for anticancer chemotherapeutic drugs. Thus, topoisomerase inhibitors, which are compounds that inhibit topoiosmerase activity, are an important category of anticancer drugs.

An example of a topoisomerase I inhibitor is an anti-tumor alkaloid, camptothecin. Camptothecin and its analogs topotecan and irinotecan are approved for clinical use. Other topoisomerase I inhibitors may also be used.

A diverse group of anti-tumor agents target DNA topoisomerase II, including epipodophyllotoxins, anthracyclines, acridines, anthracenediones ellipticines and mitroxantones (MacDonald et al., 1991, incorporated herein by reference). Under the influence of such inhibitory drugs, topoisomerase II is believed to cleave DNA and form a concomitant covalent association with the broken strand(s) of duplex DNA. The formation of such “cleavable complexes” of inhibitor, DNA and topoisomerase II enzyme has been attributed to the stabilization by the inhibitor of a covalent, DNA-bound catalytic intermediate in the cleavage-resealing resealing sequence of the enzyme.

Etoposide is an example of a epipodophyllotoxin, a widely-used antineoplastic agent which inhibits mammalian DNA topoisomerase II isoenzymes (Drake et al., 1989; Watt and Hickson, 1994; and Pommier, 1993). Various etoposide derivatives have also been developed in order to improve anti-tumor activity, cytotoxicity against drug resistant cells and drug-formulation formulation characteristics including the 4′-O-demethylepipodophyllotoxins (Zhang and Lee, 1994, incorporated herein by reference). Various other topoisomerase II inhibitors are exemplified by the procyanidins (described in U.S. Pat. No. 6,156,791, incorporated herein by reference); azatoxin and its derivatives (described in U.S. Pat. No. 5,747,520, incorporated herein by reference); colchicine derivatives (described in U.S. Pat. No. 5,639,793, incorporated herein by reference). Yet other examples of inhibitors of both topoisomerase I and topoisomerase II are described in U.S. Pat. No. 6,207,673 also incorporated herein by reference.

However, not all topoisomerase inhibitors are topoisomerase-type specific. For example, a 7-H-benzopyrido-(4,3-β)indole-derivative (inotoplicine), inhibits both topoisomerases I and II simultaneously and circumvents some topoisomerase-mediated mechanisms of drug-resistance (Podderin et al., 1993).

In addition, numerous small molecule inhibitors of topoisomerases have been identified and synthesized recently. Some examples are XR11576 (an angular benzophenazine; Vicker et al., 2002; Mistry et al., 2002), and XR5944 (Stewart et al., 2001), manufactured by Xenova Research, U.K., which are dual topoisomerase inhibitors designed for the treatment of solid tumors. Several other second and third generation small molecule inhibitors are known and the present invention contemplates the use of such compounds in the methods.

In addition, liposomal formulations and other formulations designed for easy uptake of the chemotherapeutic agents are also contemplated as especially useful.

The present invention provides that the expression of CD26 and/or exhibition of the DPPIV enzyme activity enhances the sensitivity of cells to chemotherapeutic agents. As topoisomerase inhibitors, are an important class of chemotherapeutic agents their use in conjunction with CD26 is set forth herein. Moreover, as CD26 is known to specifically increase the levels of topoisomerase II alpha, the use of topoisomerase II inhibitors is also provided. However, it will be appreciated by one of skill in the art that the present invention is not limited to the use of the specific topoisomerase inhibitors discussed here and that any other inhibitor of a topoisomerase enzyme may also be used in the practice of the present invention. Furthermore, the present invention is not limited to the use of chemotherapeutic agents that are merely inhibitors of topoisomerase. Increasing topoisomerase II levels is only one of the varied effects of CD26. Therefore, other classes of chemotherapeutic agents may also be used. A discussion of other types of chemotherapeutic agents is presented in the sections below

### C. OTHER CHEMOTHERAPEUTIC AGENTS

The present invention provides that the expression of CD26 peptides or proteins enhances the sensitivity of a cancer cell to chemotherapeutic agents. Chemotherapeutic agents are molecules that damage DNA. These can be, for example, agents that directly cross-link DNA, agents that intercalate into DNA, and agents that lead to chromosomal and mitotic aberrations by affecting nucleic acid synthesis. Agents that damage DNA also include compounds that interfere with DNA replication, mitosis, and chromosomal segregation.

Examples of chemotherapeutic agents that are contemplated as useful to the practice of the present invention include antibiotic chemotherapeutics such as, doxorubicin, liposomal doxorubicin, daunorubicin, mitomycin (also known as mutamycin and/or mitomycin-C), actinomycin D (dactinoinycin), bleomycin, plicomycin; plant alkaloids such as taxol, vincristine, vinblastine; alkylating agents such as, carmustine, melphalan (also known as alkeran, L-phenylalanine mustard, phenylalanine mustard, L-PAM, or L-sarcolysin, is a phenylalanine derivative of nitrogen mustard), cyclophosphamide, chlorambucil, busulfan (also known as myleran), lomustine; and miscellaneous agents such as visplatin, etoposide (VP16), tumor necrosis factor, cisplatin (CDDP), carboplatin, procarbazine, mechlorethamine, camptothecin, ifosfamide, nitrosurea, tamoxifen, raloxifene, estrogen receptor binding agents, gemcitabien, navelbine, famesyl-protein transferase inhibitors, transplatinum, 5-fluorouracil, methotrexate, temazolomide (an aqueous form of DTIC), or any analog or derivative variant of the foregoing. In addition, liposomal formulations and other formulations designed for easy uptake of the chemotherapeutic agents are also contemplated as especially useful.

### D. RADIOTHERAPEUTIC AGENTS

The methods of the invention also provide that treatment of a cell with a CD26 composition increase the sensitivity of a cell to radiotherapeutic agents. Radiotherapeutic agents include radiation and waves that induce DNA damage for example, γ-irradiation, X-rays, UV-irradiation, microwaves, electronic emissions, radioisotopes, and the like. Therapy may be achieved by irradiating the localized tumor site with the above described forms of radiations. It is most likely that all of these factors effect a broad range of damage DNA, on the precursors of DNA, the replication and repair of DNA, and the assembly and maintenance of chromosomes.

Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

In the methods of the present invention, it is envisioned that the radiotherapeutic agent may be further conjugated to a targeting agent and/or the CD26 composition. This in one embodiment, the invention provides a biomolecule that can target a tumor, for example, an antibody against a tumor marker, a ligand that binds to a growth factor or other receptor molecule that is differentialy expressed by tumor cells, etc., that is conjugated to a CD26 composition as well as a radiotherapeutic agent.

### E. VECTORS FOR EXPRESSION OF CD26 AND FRAGMENTS THEREOF

Within certain embodiments, expression constructs (also referred to as expression vectors), are employed to express a CD26 peptide or protein product. In some embodiments the expression vectors encode an enzymatically active fragment of CD26 which exhibits the DPPIV activity. Specifically, it is contemplated that the expression vectors/constructs of the invention may encode for DNA sequences encoded by SEQ ID NO. 1; SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID-NO: 21, SEQ ID NO: 22, SEQ ID NO: 23, SEQ ID NO: 24, SEQ ID-NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 36, and/or GenBank Accession Numbers M74777, NM_{—}001935, AH005372, XM_{—}02930, BC133029, NM_{—}010074, AH003239, AF461806, (GenBank Accession Numbers incorporated herein by reference), and/or any fragment, variant, isoform, mutations or biologically functional equivalents thereof and/or any other CD26 sequence. The expression vectors/constructs of the invention may also encode for a peptide or protein having SEQ. ID NO.2, SEQ ID NO: 4, SEQ ID NO: 33, SEQ ID NO: 35, SEQ ID NO: 37, and/or encoded by GenBank Accession Numbers M74777, NM_{—}001935, AH005372, XM_{—}02930, BC133029, NM_{—}010074, AH003239, AF461806, and/or any fragment, isoform, variant, mutation, biologically functionally equivalent, or mimetic thereof.

Various nucleic acid segments may be designed based on a particular nucleic acid sequence, and may be of any length. By assigning numeric values to a sequence, for example, the first residue is 1, the second residue is 2, etc., an algoritlun defining all nucleic acid segments can be created:
n to n+y

where n is an integer from 1 to the last number of the sequence and y is the length of the nucleic acid segment minus one, where n+y does not exceed the last number of the sequence. Thus, for a 10-mer, the nucleic acid segments correspond to bases 1 to 10, 2 to 11, 3 to 12 . . . and/or so on. For a 15-mer, the nucleic acid segments correspond to bases 1to 15, 2 to 16, 3 to 17 . . . and/or so on. For a 20-mer, the nucleic segments correspond to bases 1 to 20, 2 to 21, 3 to 22 . . . and/or so on.

In context of the present invention, the CD26 encoding nucleic acid(s), regardless of the length of the sequence itself, may be combined with other nucleic acid sequences, including but not limited to, promoters, enhancers, polyadenylation signals, restriction enzyme sites, multiple cloning sites, coding segments, and the like, to create one or more nucleic acid construct(s). The overall length may vary considerably between nucleic acid constructs. Thus, a nucleic acid segment of almost any length may be employed, with the total length preferably being limited by the ease of preparation or use in the intended recombinant nucleic acid protocol.

In a non-limiting example, one or more nucleic acid constructs may be prepared that include a contiguous stretch of nucleotides identical to or complementary to SEQ ID NO:1 or any of the other sequences described above. Such a stretch of nucleotides, or a nucleic acid construct, may be or be about 10, about 15, about 20, about 25, about 30, about 35, about 40, about 45, about 50, about 55, about 60, about 65, about 70, about 75, about 80, about 85, about 90, about 95, about 100, about 105, about 110, about: 115, about 120, about 125, about 130, about 135, about 140, about 145, about 150, about 155, about 160, about 165, about 170, about 175, about 180, about 185, about 190, about 195, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, about 600, about 610, about 620, about 630, about 640, about 650, about 660, about 670, about 680, about 690, about 700, about 710, about 720, about 730, about 740, about 750, about 760, about 770, about 780, about 790, about 800, about 810, about 820, about 830, about 840, about 850, about 860, about 480, about 880, about 890, about 900, about 910, about 920, about 930, about 940, about 950, about 960, about 970, about 980, about 990, about 1,000, about 1010, about 1020, about 1030, about 1040, about 1050, about 1060, about 1070, about 1080, about 1090, about 1,100, about 1110, about 1120, about 1130, about 1140, about 1150, about 1160, about 1170, about 1180, about 1190, about 1200, 1210, about 1220, about 1230, about 1240, about 1250, about 1260, about 1270, about 1280, about 1290, about 2000, about 2100, about 2200, about 2300 to about 2311, nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges,” as used herein, means any length or range including or between the given values (i.e., all integers including and between such values).

A biologically functional equivalent is molecule where modifications and/or changes may be made in the structure of the polynucleotides and and/or proteins encoding the molecule, while obtaining molecules having similar or improved characteristics. In the context of vectors encoding nucleic acids a biological functional equivalent may comprise a polynucleotide that has been engineered to contain distinct sequences while at the same time retaining the capacity to encode the “wild-type” or standard protein. This can be accomplished to the degeneracy of the genetic code, i.e., the presence of multiple codons, which encode for the same amino acids. Methods for preparing such equivalents are well known in the art.

Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

(i) Regulatory Elements

Throughout this application, the term “expression construct” or “expression vector” is meant to include any type of genetic construct containing a nucleic acid coding for a gene, product in which part or all of the nucleic acid encoding sequence is capable of being transcribed and translated into a polypeptide product. The nucleic acid encoding the gene product in a expression construct is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In certain embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Further, selection of a promoter that is regulated in response to specific physiologic signals can permit inducible expression of the gene product. Tables 2 and 3 list several regulatory elements that may be employed, in the context of the present invention, to regulate the expression of the gene of interest. This list is not intended to be exhaustive of all the possible elements involved in the promotion of gene expression but, merely, to be exemplary thereof.

Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of viral promoters, cellular promoters/enhancers and inducible promoters/enhancers that could be used in combination with the nucleic acid encoding a gene of interest in an expression construct (Table 1 and Table 2). Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

TABLE 1
Promoter and/or Enhancer

| Promoter/Enhancer | References |
|---|---|
| Immunoglobulin Heavy Chain | Banerji et al., 1983; Gilles et al., 1983; Grosschedl et |
| | al., 1985; Atchinson et al., 1986, 1987; Imler et al., |
| | 1987; Weinberger et al., 1984; Kiledjian et al., |
| | 1988; Porton et al.; 1990 |
| Immunoglobulin Light Chain | Queen et al., 1983; Picard et al., 1984 |
| T-Cell Receptor | Lurina et al., 1987; Winoto et al., 1989; Redondo et |
| | al.; 1990 |
| HLA DQ a and/or DQ | Sullivan et al., 1987 |
| Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| Actin | Kawamoto et al., 1988; Ng et al.; 1989 |
| Muscle Creatine Kinase (MCK) | Jaynes et al., 1988; Horlick et al., 1989; Johnson et |
| | al., 1989 |
| Prealbumin (Transthyretin) | Costa et al., 1988 |
| Elastase I | Ornitz et al., 1987 |
| Metallothionein (MTII) | Karin et al., 1987; Culotta et al., 1989 |
| Collagenase | Pinkert et al., 1987; Angel et al., 1987a |
| Albumin | Pinkert et al., 1987; Tronche et al., 1989, 1990 |
| Fetoprotein | Godbout et al., 1988; Campere et al., 1989 |
| t-Globin | Bodine et al., 1987; Perez-Stable et al., 1990 |
| Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| ₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et |
| | al., 1985; Firak et al., 1986; Herr et al., 1986; Imbra |
| | et al., 1986; Kadesch et al., 1986; Wang et al., 1986; |
| | Ondek et al., 1987; Kuhl et al., 1987; Schaffner et |
| | al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; Hen et |
| | al., 1986; Satake et al., 1988; Campbell and/or |
| | Villarreal, 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Choi et |
| | al., 1988; Reisman et al., 1989 |
| Papilloma Virus | Campo et al., 1983; Lusky et al., 1983; Spandidos |
| | and/or Wilkie, 1983; Spalholz et al., 1985; Lusky et |
| | al., 1986; Cripe et al., 1987; Gloss et al., 1987; |
| | Hirochika et al., 1987; Stephens et al., 1987 |
| Hepatitis B Virus | Bulla et al., 1986; Jameel et al., 1986; Shaul et al., |
| | 1987; Spandau et al., 1988; Vannice et al., 1988 |
| Human Immunodeficiency Virus | Muesing et al., 1987; Hauber et al., 1988; Jakobovits |
| | et al., 1988; Feng et al., 1988; Takebe et al., 1988; |
| | Rosen et al., 1988; Berkhout et al., 1989; Laspia et |
| | al., 1989; Sharp et al., 1989; Braddock et al., 1989 |
| Cytomegalovirus (CMV) | Weber et al., 1984; Boshart et al., 1985; Foecking et |
| | al., 1986 |
| Gibbon Ape Leukemia Virus | Holbrook et al., 1987; Quinn et al., 1989 |

TABLE 2
Inducible Elements

| Element | Inducer | References |
|---|---|---|
| MT II | Phorbol Ester (TFA) | Palmiter et al., 1982; |
| | Heavy metals | Haslinger et al., 1985; |
| | | Searle et al., 1985; Stuart et |
| | | al., 1985; Imagawa et al., |
| | | 1987, Karin et al., 1987; |
| | | Angel et al., 1987b; |
| | | McNeall et al., 1989 |
| MMTV (mouse mammary | Glucocorticoids | Huang et al., 1981; Lee et |
| tumor virus) | | al., 1981; Majors et al., |
| | | 1983; Chandler et al., 1983; |
| | | Ponta et al., 1985; Sakai et |
| | | al., 1988 |
| Interferon | poly(rI)x | Tavernier et al., 1983 |
| | poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| CRP | IL-6, IL-1 | Ku & Mortensen, 1993 |
| SAA | IL-6, IL-1 | Jiang et al., 1995 |
| SV40 | Phorbol Ester (TPA) | Angel et al., 1987b |
| Murine MX Gene | Interferon, Newcastle Disease | Hug et al., 1988 |
| | Virus | |
| GRP78 Gene | A23187 | Resendez et al., 1988 |
| -2-Macroglobulin | IL-6 | Kunz et al., 1989 |
| Vimentin | Serum | Rittling et al., 1989 |
| MHC Class I Gene H-2b | Interferon | Blanar et al., 1989 |
| HSP70 | ElA, SV40 Large T Antigen | Taylor et al., 1989, 1990a, |
| | | 1990b |
| Proliferin | Phorbol Ester-TPA | Mordacq et al., 1989 |
| Tumor Necrosis Factor | TPA | Hensel et al., 1989 |
| Thyroid Stimulating | Thyroid Hormone | Chatterjee et al., 1989 |
| Hormone Gene | | |

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

(ii) Selectable Markers

In certain embodiments of the invention, the cells contain nucleic acid constructs of the present invention, a cell may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

(iii) Polyadenylation Signals

In expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Also contemplated as an element of the expression cassette is a transcriptional termination site. These elements can serve to enhance message levels and/or to minimize read through from the cassette into other sequences.

(iv) Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosurids, viruses (bacteriophage, animal viruses, and plant viruses), and artificial chromosomes (e.g., YACs). One of skill in the art would be well equipped to construct a vector through standard recombinant techniques, which are described in Sambrook et al. (1989) and Ausubel et al. (1994), both incorporated herein by reference.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

(v) Delivery of Expression Vectors

There are a number of ways in which expression vectors may be introduced into cells. In certain embodiments of the invention, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubenstein, 1988; Temin, 1986).

Adenovirus. One of the methods for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete. For example, leakage of viral gene expression has been observed with the currently available vectors at high multiplicities of infection (MOI) (Mulligan, 1993).

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell innoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors, as described by Karlsson et al. (1986), or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹² plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1991). Recently, animal studies suggested that recombinant adenovirus could be used for gene therapy (Stratford-Perricaudet & Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

Retrovirus. The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et. al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell-receptor were used. The antibodies were coupled via the biotin components by using streptavidin (Roux et. al., 1989). Using antibodies against major histocompatibility complex class I and class II antigens, they demonstrated the infection of a variety of human cells that bore those surface antigens with an ecotropic virus in vitro (Roux et al., 1989).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Vannus et al., 1981). Another concern with the use of defective retrovirus vectors is the potential appearance of wild-type replication-competent virus in the packaging cells. This can result from recombination events in which the intact-sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Adeno-Associated Viruses. Adeno-associated virus (AAV) is an attractive virus for delivering foreign genes to mammalian subjects (Ridgeway, 1988; Baichwal and Sugden, 1986; Hermonat and Muzycska, 1984). AAV utilizes a linear, single-stranded DNA of about 4700 base-pairs. Inverted terminal repeats flank the genome. Two genes are present within the genome, giving rise to a number of distinct gene products. The first, the cap gene, produces three different virion proteins (VP), designated VP-1, VP-2 and VP-3. The second, the rep gene, encodes four non-structural proteins (NS). One or more of these rep gene products is responsible for transactivating AAV transcription. The sequence of AAV is provided by U.S. Pat. No. 5,252,479 (entire text of which is specifically incorporated herein by reference).

The three promoters in AAV are designated by their location, in map units, in the genome. These are, from left to right, p5, p19 and p40. Transcription gives rise to six transcripts, two initiated at each of three promoters, with one of each pair being spliced. The splice site, derived from map units 42-46, is the same for each transcript. The four non-structural proteins apparently are derived from the longer of the transcripts, and three virion proteins all arise from the smallest transcript.

AAV is not associated with any pathologic state in humans. Interestingly, for efficient replication, AAV requires “helping” functions from viruses such as herpes simplex virus I and II, cytomegalovirus, pseudorabies virus and, of course, adenovirus. The best characterized of the helpers is adenovirus, and many “early” functions for this virus have been shown to assist with AAV replication. Low level expression of AAV rep proteins is believed to hold AAV structural expression in check, and helper virus infection is thought to remove this block.

The terminal repeats of the AAV vector of the present invention can be obtained by restriction endonuclease digestion of AAV or a plasmid such as p201, which contains a modified AAV genome (Samulski et al., 1987). Alternatively, the terminal repeats may be obtained by other methods known to the skilled artisan, including but not limited to chemical or enzymatic synthesis of the terminal repeats based upon the published sequence of AAV. The ordinarily skilled artisan can determine, by well-known methods such as deletion analysis, the minimum sequence or part of the AAV ITRs which is required to allow function, i.e., stable and site-specific integration. The ordinarily skilled artisan also can determine which minor modifications of the sequence can be tolerated while maintaining the ability of the terminal repeats to direct stable, site-specific integration.

Other Viruses. Other viral vectors may be employed as expression constructs in the present invention. Vectors derived from viruses such as vaccinia virus (Ridgeway, 1988; Baichwal and Sugden, 1986; Coupar et al., 1988), herpesviruses, and lentiviruses, may be employed. They offer several attractive features for various mammalian cells (Friedmann, 1989; Ridgeway, 1988; Baichwal and Sugden, 1986; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. The hepatotropism and persistence (integration) were particularly attractive properties for liver-directed gene transfer. Chang et al. recently introduced the chloramphenicol acetyltransferase (CAT) gene into duck hepatitis B virus genome in the place of the polymerase, surface, and pre-surface coding sequences. It was co-transfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

Non-Viral Methods. Several non-viral methods for the transfer of expression constructs into mammalian cells also are contemplated by the present invention. These include DEAE-dextran (Gopal; 1985), electroporation (Tur-Kaspa et al., 1986; Potter et al., 1984), direct microinjection (Harland and Weintraub, 1985), lipofectamine-DNA complexes, cell sonication (Fechheimer et al., 1987), and receptor-mediated transfection (Wu and Wu, 1987; Wu and Wu, 1988).

In yet another embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

Liposomes. In a further embodiment of the invention, the expression construct encoding a CD26 peptide or protein may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are Lipofectamine-DNA complexes.

Liposome-mediated nucleic acid delivery and expression of foreign DNA in vitro has been very successful. Wong et al. (1980) demonstrated the feasibility of liposome-mediated delivery and expression of foreign DNA in cultured chick embryo, HeLa and hepatoma cells. Nicolau et al. (1987) accomplished successful liposome-mediated gene tasfer in rats after intravenous injection.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol. et al., 1993; Perales et al., 1994) and epidermal growth factor (EGF) has also been used to deliver genes to squamous carcinoma cells (Myers, EPO 0 273 085).

In other embodiments, the delivery vehicle may comprise a ligand and a liposome. For example, Nicolau et al. (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type by any number of receptor-ligand systems with or without liposomes. For example, epidermal growth factor (EGF) may be used as the receptor for mediated delivery of a nucleic acid into cells that exhibit upregulation of EGF receptor. Mannose can be used to target the mannose receptor on liver cells.

### F. CD26 PEPTIDES AND PROTEINS

CD26 peptides and/or proteins may be directly provided to a cancer cell or an immune cell, such as an APC, according to the therapeutic methods of the present invention. In some embodiments a full-length or a substantially full-length or a fragment of a CD26 polypeptide may be used. The term “full-length” refers to a CD26 polypeptide that encodes the entire CD26 protein such as that encoded by all the amino acids of SEQ. ID NO.2 (766 amino acids); SEQ. ID NO.4; SEQ. ID NO.33 (688 amino acids); SEQ. ID NO.35; SEQ. ID NO.37; and/or GenBank Accession Numbers M74777, NM₁₃ 001935, AH005372, XM_{—}02930, BC133029, NM_{—}010074, AH003239, or AF461806, (GenBank numbers incorporated herein by reference), and/or any variant, isoform, or mutation thereof. Alternatively, the CD26 protein or peptide may be any fragment, domain, variant, or mutation of the amino acid sequence encoded by SEQ. ID NO.2; SEQ. ID NO.4; SEQ. ID NO.33; SEQ. ID NO.35; SEQ. ID NO.37; and/or GenBank Accession Numbers M74777, NM_{—}001935, AH005372, XM_{—}02930, BC133029, NM_{—}010074, AH003239, or AF461806. Soluble forms of the CD26 protein and fragments with the ability to exhibit the enzymatic DPPIV activity are also contemplated as useful.

The invention also contemplates the use of a biologically functional equivalent of a CD26 protein or peptide. The terms “CD26 protein” “CD26 peptide,” or “CD26 polypeptide” is used herein to refer to a CD26 protein or peptide, irrespective of whether the it occurs naturally, is substantially purified, is partially purified, or is produced by recombinant DNA methods, fusion-protein methods, protein synthesis methods, etc., or is a biological functional equivalent, mimetic or derivative thereof.

The term “biologically functional equivalent” is well understood in the art and is further defined in detail herein. Accordingly, a sequence that has between about 70% and about 80%; or more preferably, between about 81% and about 90%; or even more preferably, between about 91% and about 99%; of amino acids that are identical or functionally equivalent to the amino acids of CD26 encoding amino acid sequences such as the ones described above will be a sequence that is “essentially as set forth” in these sequences provided the biological activity of the protein, polypeptide, or peptide is maintained.

The term “functionally equivalent codon” is used herein to refer to codons that encode the same amino acid, such as the six codons for arginine and serine, and also refers to codons that encode biologically equivalent amino acids (see Table 5).

Excepting intronic and flanking regions, and allowing for the degeneracy of the genetic code, nucleic acid sequences that have between about 70% and about 79%; or more preferably, between about 80% and about 89%; or even more particularly, between about 90% and about 99%; of nucleotides that are identical to the nucleotides of the CD26 sequences set forth above, will be nucleic acid sequences that are “essentially as set forth” in these CD26 sequences.

It will also be understood that this invention is not limited to the particular nucleic acid and amino acid sequences of CD26 descnbed above. Recombinant vectors and isolated nucleic acid segments may therefore variously include these coding regions themselves, coding regions bearing selected alterations or modifications in the basic coding region, and they may encode larger polypeptides or peptides that nevertheless include such coding regions or may encode biologically functional equivalent proteins, polypeptide or peptides that have variant amino acids sequences.

The nucleic acids of the present invention encompass biologically functional equivalent CD26 proteins or peptides. Such sequences may arise as a consequence of codon redundancy or functional equivalency that are known to occur naturally within nucleic acid sequences or the proteins or peptides thus encoded. Alternatively, functionally equivalent proteins or peptides may be created via the application of recombinant DNA technology, in which changes in the protein, polypeptide or peptide structure may be engineered, based on considerations of the properties of the amino acids being exchanged. Recombinant changes may be introduced, for example, through the application of site-directed mutagenesis techniques as discussed herein below, e.g., to introduce improvements or alterations to the antigenicity of the protein or peptide, or to test mutants in order to examine CD26 protein, polypeptide, or peptide activity at the molecular level.

Fusion proteins, polypeptides or peptides may be prepared, e.g., where the CD26 coding regions are aligned within the same expression unit with other proteins or peptides having desired functions. Non-limiting examples of such desired functions of expression sequences include purification or immunodetection or even target recognition purposes for the added expression sequences, e.g., proteinaceous compositions that may be purified by affinity chromatography or the enzyme labeling of coding regions, respectively or targeting to a cancer or an immune cell by making a fusion with a molecule that recognizes and binds to such a cell.

(i) Fragments

The present invention also provides that fragments of the CD26 polypeptide that may retain their anti-tumor or immune potentiating properties. Such fragments may be exemplified by the enzymatic DPPIV domain, or any other domain, and may be generated by genetic engineering of translation stop sites within the coding region (discussed below). Alternatively, treatment of the CD26 molecule with proteolytic enzymes, known as proteases, can produces a variety of N-terminal, C-terminal and internal fragments. Examples of fragments may include contiguous residues of the CD26 sequence given in SEQ ID NO:2, SEQ ID NO:4, SEQ ID NO:33, SEQ ID NO:35, SEQ ID NO: 37, and/or GenBank Accession Numbers M74777, NM_{—}001935, AH005372, XM_{—}02930, BC133029, AH003239, and AF461806, of 5, 6, 7, 8, 9, 10, 11, 12, 13, 14, 15, 16, 17, 18, 19, 20, 21, 22, 23, 24, 25, 30, 35, 40, 45, 50, 55, 60, 65, 75, 80, 85, 90, 95, 100, 125, 150, 175, 200, 225, 250, 275, 300, 325, 350, 375, 400, 425, 450, 475, 500, 525, 550, 575, 600, 625, 650, 675, 688, 700, 725, 750, 766, 775, 800, 825, 850, 875, 900, 925, 950, 975, 1000 or more amino acids in length. Intermediate lengths are also contemplated and exemplified by 110, 209, 306, 555, amino acids. These fragments may be purified according to known methods, such as precipitation (e.g., ammonium sulfate), HPLC, ion exchange chromatography, affinity chromatography (including immunoaffinity chromatography) or various size separations (sedimentation, gel electrophoresis, gel filtration).

(ii) Variants and Mutants of CD26

Amino acid sequence variants of CD26 also are encompassed by the present invention. Amino acid sequence variants of the polypeptide can be substitutional, insertional or deletion variants. Deletion variants lack one or more residues of the native protein which are not essential for function or immunogenic activity, and are exemplified by the variants lacking a transmembrane sequence. Soluble formulations of CD26 are contemplated as useful and as CD26 is a membrane protein variants that lack the transmembrane domain are contemplated. Another common type of deletion variant is one lacking secretory signal sequences or signal sequences directing a protein to bind to a particular part of a cell. Insertional mutants typically involve the addition of material at a non-terminal point in the polypeptide. This may include the insertion of an immunoreactive epitope or simply a single residue. Terminal additions, called fusion proteins, are discussed below.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tytosine, leucine or methionine; serine to threonine; threonine to serine; tryptophan to tyrosine; tyrosine to tryptophan or phenylalanine; and valine to isoleucine or leucine.

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and its underlying DNA coding sequence, and nevertheless obtain a protein with like properties. It is thus contemplated that various changes may be made in the DNA sequences of genes without appreciable loss of their biological utility or activity, as discussed below. Table 3 shows the codons that encode particular amino acids.

TABLE 3

CODON TABLE

| | |
|---|---|
| Amino Acids | Codons |

| | | | | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Typtophan | Trp | W | UGG | | | | | |
| Tyrosine | Tyr | Y | UAC | UAU | | | | |

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those which are within ±1 are particularly preferred, and those within ±0.5 are even more particularly preferred.

It also is understood in the art that the substitution of like amino acids can be made effectively on the basis of hydrophilicity. U.S. Pat. No. 4,554,101, incorporated herein by reference, states that the greatest local average hydrophi licity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtain a biologically equivalent and immunologically equivalent protein. In such changes, the substitution of amino acids whose hydrophilicity values are within ±2 is preferred, those that are within ±1 are particularly preferred, and those within ±0.5are even more particularly preferred.

As outlined above, amino acid-substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutanine and asparagine; and valine, leucine and isoleucine.

Another embodiment for the preparation of polypeptides according to the invention is the use of peptide mimetics. Mimetics are peptide-containing molecules that mimic elements of protein secondary structure. See, for example, Johnson et al., (1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of CD26, but with altered and even improved characteristics.

(iii) Fusion Proteins

A specialized kind of insertional variant is the fusion protein. This molecule generally has all or a substantial portion of the native molecule, linked at the N— or C-terminus, to all or a portion of a second polypeptide. For example, fusions typically employ leader sequences from other species to permit the recombinant expression of a protein in a heterologous host. Another useful fusion includes the addition of a immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, or cellular targeting signals. Cellular targeting signals to target CD26 proteins or peptides to cancer cells or immune cells are an important aspect of the invention. Furthermore, fusion to a polypeptide that can be used for purification of the substrate-CD26 complex would serve to isolated the substrate for identification and analysis.

Examples of such fusion protein expression systems are the glutathione S-transferase (GST) system (Pharmacia, Piscataway, N.J.), the maltose binding protein system (NEB, Beverley, Mass.), the FLAG system (IBI, New Haven, Conn.), and the 6xHis system (Qiagen, Chatsworth, Calif.).

Some of these systems produce recombinant polypeptides bearing only a small number of additional amino acids, which are unlikely to affect the antigenic ability of the recombinant polypeptide. For example, both the FLAG system and the 6xHis system add only short sequences, both of which are known to be poorly antigenic and which do not adversely affect folding of the polypeptide to its native conformation.

In other embodiments, fusion construct can be made which will enhance the targeting of the CD26 related compositions to a specific site or cell, such as a cancer cell or a immune cell For example, fusing CD26 or a CD26 type protein to a ligand will be an effective means to target the composition to a site expressing the receptor for such a ligand. In this manner the CD26 or CD26 related composition may be delivered into a cell via receptor mediated delivery. CD26 can be attached covalently or fused to a ligand. This can be used as a mechanics for delivery into a cell. The ligand with the CD26 attached may then be internalized by a receptor bearing cell.

Other fusion systems produce polypeptide hybrids where it is desirable to excise the fusion partner from the desired polypeptide. In one embodiment, the fusion partner is linked to the recombinant CD26 cancer polypeptide by a peptide sequence containing a specific recognition sequence for a protease. Examples of suitable sequences are those recognized by the Tobacco Etch Virus protease (Life Technologies, Gaithersburg, Md.) or Factor Xa (New England Biolabs, Beverley, Mass.).

As CD26 has transmembrane sequences which are often deleterious when a recombinant protein is synthesized in many expression systems, especially E. coli, as it leads to the production of insoluble aggregates that are difficult to renature into the native conformation of the protein. Deletion of transmembrane sequences typically does not significantly alter the conformation of the remaining protein structure. Deletion of transmembrane-encoding sequences from the genes used for expression can be achieved by standard techniques. For example, fortuitously-placed restriction enzyme sites can be used to excise the desired gene fragment, or PCR-type amplification can be used to amplify only the desired part of the gene. Thus, soluble versions of CD26 proteins or peptides ma be obtained.

(iv) Synthetic Peptides

The present invention also describes smaller CD26-related peptides for use in various embodiments of the present invention. Such peptides should generally be at least five or six amino acid residues in length, and may contain up to about 35-50 residues or so. Because of their relatively small size, the peptides of the invention can also be: synthesized in solution or on a solid support in accordance with conventional techniques.

Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Short peptide sequences, or libraries of overlapping peptides, usually from about 6 up to about 35 to 50 amino-acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

U.S. Pat. No. 4,554,101 (Hopp, incorporated herein by reference) also teaches the identification and preparation of epitopes from primary amino acid sequences on the basis of hydrophilicity. Through the methods disclosed in Hopp, one of skill in the art would be able to identify epitopes from within any amino acid sequence encoded by any of the DNA sequences disclosed herein.

(v) Recombinant Protein Expression

Irrespective of whether a full length, unmodified protein, a variant, a fusion protein, or a peptide of CD26 is used, recombinant vectors evidently form further important aspects of the present invention.

The vectors will generally have the coding portion of the DNA segment, whether encoding a full length protein or smaller peptide, positioned under the control of a promoter. The promoter may be in the form of the promoter that is naturally associated with a CD26 gene, e.g., in a variety of cancer cells including melanoma, glioblastomas, astrocytomas and carcinomas of the breast, gastric, colon, pancreas, renal, ovarian, lung, prostate, hepatic, and lung cells, and hematological cancer cells, as may be obtained by isolating the non-coding sequences located upstream of the coding segment or exon, for example, using recombinant cloning and/or PCR technology, (PCR technology is disclosed in U.S. Pat. No. 4,683,202 and U.S. Pat. No. 4,682,195, each incorporated herein by reference).

In other embodiments, it is contemplated that certain advantages will be gained by positioning the coding DNA segment under the control of a recombinant, or heterologous, promoter. As used herein, a recombinant or heterologous promoter is intended to refer to a promoter that is not normally associated with a CD26 gene in its natural environment. Such promoters may include tissue promoters normally associated with other genes, and/or promoters isolated from any other bacterial, viral, eukaryotic, or mammalian cell.

Naturally, it will be important to employ a promoter that effectively directs the expression of the DNA segment in the cell type, organism, or even animal, chosen for expression. The use of promoter and cell type combinations for protein expression is generally known to those of skill in the art of molecular biology, for example, see Sambrook et al. (2001). The promoters employed may be constitutive, or inducible, and can be used under the appropriate conditions to direct high level expression of the introduced DNA segment, such as is advantageous in the large-scale production of recombinant proteins or peptides.

The preparation and engineering of expression vectors for use in prokaryotic or eukaryotic systems is well known to those of skill in the art. It is believed that virtually any expression vector and system may be employed in the expression of one or more antigens of this invention.

Both cDNA and genomic sequences are suitable for eukaryotic expression, as the host cell will generally process the genomic transcripts to yield functional mRNA for translation into protein. Generally speaking, it may be more convenient to employ as the recombinant gene a cDNA version of the gene. It is believed that the use of a cDNA version will provide advantages in that the size of the gene will generally be much smaller and more readily employed to transfect the targeted cell than will a genomic gene, which will typically be up to an order of magnitude larger than the cDNA gene. However, the present invention do not exclude the possibility of employing a genomic version of a particular gene where desired.

As used herein, the terms “engineered” and “recombinant” cells are intended to refer to a cell into which an exogenous DNA segment or gene, such as a cDNA or gene encoding CD26, has been introduced. Therefore, engineered cells are distinguishable from naturally occurring cells which do not contain a recombinantly introduced exogenous DNA segment or gene. Engineered cells are thus cells having a gene or genes introduced through the hand of man. Recombinant cells include those having an introduced cDNA or genomic gene, and also include genes positioned adjacent to a promoter not naturally associated with the particular introduced gene.

To express a recombinant CD26, in accordance with the present invention, one would prepare an expression vector that comprises the coding nucleic acid under the control of one or more promoters. To bring a coding sequence “under the control of” a promoter, one positions the 5 end of the transcription initiation site of the transcriptional reading frame generally between about 1 and about 50 nucleotides “downstream” of (i.e., 3 of) the chosen promoter. The “upstream” promoter stimulates transcription of the DNA and promotes expression of the encoded recombinant protein. This is the meaning of “recombinant expression” in this context.

Many standard techniques are available to construct expression vectors containing the appropriate nucleic acids and transcriptional/translational control sequences in order to achieve protein or peptide expression in a variety of host-expression systems. Cell types available for expression include, but are not limited to, bacteria, such as E. coli and B. subtilis transformed with recombinant bacteriophage DNA, plasmid DNA or cosmid DNA expression vectors.

Certain examples of prokaryotic hosts are E. coli strain RR1, E. coli LE392, E. coli B, E. coli X 1776 (ATCC No. 31537) as well as E. coli W3110 (F-, lambda-, prototrophic, ATCC No. 273325); bacilli such as Bacillus subilis; and other enterobacteriaceae such as Salmonella typhimurium, Serratia marcescens, and various Pseudomonas species.

In general, plasmid vectors containing replicon and control sequences which are derived from species compatible with the host cell are used in connection with these hosts. The vector ordinarily carries a replication site, as well as marking sequences which are capable of providing phenotype selection in transformed cells. For example, E. coli is often transformed using pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, promoters which can be used by the microbial organism for expression of its own proteins.

In addition, phage vectors containing replicon and control sequences that are compatible with the host microorganism can be used as transforming vectors in connection with these hosts. For example, the phage lambda GEM™-11 may be utilized in making a recombinant phage vector which can be used to transform host cells, such as E. coli LE392.

Further useful vectors include pIN vectors (Inouye et al., 1985); and pGEX vectors, for use in generating glutathione S-transferase (GST) soluble fusion proteins for later purification and separation or cleavage. Other suitable fusion proteins are those with—galactosidase, ubiquitin, the like.

Promoters that are most commonly used in recombinant DNA construction include the β-lactamase (penicillinase), lactose and tryptopban (trp) promoter systems. While these are the most commonly used, other microbial promoters have been discovered and utilized, and details concerning their nucleotide sequences have been published, enablin g those of skill in the art to ligate them functionally with plasmid vectors.

For expression in Saccharomyces, the plasmid YRp7, for example, is commonly used (Stinchcomb et al., 1979; Kingsman et al., 1979; Tschemper et al., 1980). This plasmid already contains the trp1 gene which provides a selection marker for a mutant strain of yeast lacking the ability to grow in tryptopban, for example ATCC No. 44076 or PEP4-1 (Jones, 1977). The presence of the trp1 lesion as a characteristic of the yeast host cell genome then provides an effective environment for detecting transformation by growth in the absence of tryptophan.

Suitable promoting sequences in yeast vectors include the promoters for 3-phosphoglycerate kinase (Hitzeman et al., 1980) or other glycolytic enzymes (Hess et al., 1968;Holland et al., 1978), such as enolase, glyceraldehyde-3-phosphate dehydrogenase, hexokinase, pyruvate decarboxylase, phosphofructolinase, glucose-6phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also ligated into the expression vector 3 of the sequence desired to be expressed to provide polyadenylation of the mRNA and termination.

Other suitable promoters, which have the additional advantage of transcription controlled by growth conditions, include the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization.

In addition to micro-organisms, cultures of cells derived from multicellul ar organisms also may be used as hosts. In principle, any such cell culture is workable, whether from vertebrate or invertebrate culture. In addition to mammalian cells, these include insect cell systems infected with recombinant virus expression vectors (e.g., baculovirus); and plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing one or more coding sequences.

In a useful insect system, Autograph californica nuclear polyhidrosis virus (AcNPV) is used as a vector to express foreign genes. The virus grows in Spodoptera frugiperda cells. The CD26 coding sequences are cloned into non-essential regions (for example the polyhedrin gene) of the virus and placed under control of an AcNPV promoter (for example the polyhedrin promoter). Successful insertion of the coding sequences results in the inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed (e.g., U.S. Pat. No. 4,215,051, incorporated herein by reference). A useful exemplary baculovirus vector is the pBlueBac vector (Invitrogen, Sorrento, Calif.).

Examples of useful mammalian host cell lines are VERO and HeLa cells, Chinese hamster ovary (CHO) cell lines, W138, BHK, COS-7, 293, HepG2, 3T3, RIN and MDCK cell lines. In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein, and it is reasonable to assume that cellular mechanisms modulate metastasis-related genes in response to changing times and conditions.

Different host cells have characteristic and specific mechanisms for the post-translational processing and modification of proteins. Appropriate cells lines or host systems can be chosen to ensure the correct modification and processing of the foreign protein expressed. To this end, eukaryotic host cells will often be preferred.

Expression vectors for use in such cells ordinarily include an origin of replication (as necessary), a promoter located in front of the gene to be expressed, along with any necessary ribosome binding sites, RNA splice sites, polyadenylation site, and transcriptional terminator sequences. The origin of replication may be provided either by construction of the vector to include an exogenous origin, such as may be derived from SV40 or other viral (e.g., Polyoma, Adeno, VSV, BPV) source, or may be provided by the host cell chromosomal replication mechanism. If the vector is integrated into the host cell chromosome, the latter is often sufficient.

The promoters may be derived from the genome of mammalian cells ( e.g., metallothionein promoter) or from mammalian viruses (e.g., the adenovirus late promoter, the vaccinia virus 7.5K promoter). Further, it also is possible, and may be desirable, to utilize promoter or control sequences normally associated with the desired CD26 cancer gene sequence, provided such control sequences are compatible with the host cell systems.

A number of viral based expression systems may be utilized, for example, commonly used promoters are derived from polyoma, Adenovirus 2, and most frequently Simian Virus 40 (SV40). The early and late promoters of SV40 virus are particularly useful because both are obtained easily from the virus as a fragment which also contains the SV40 viral origin of replication. Smaller or larger SV40 fragments also may be used, provided there is included the approximately 250 bp sequence extending from the HindIII site toward the BglI site located in the viral origin of replication.

In cases where an adenovirus is used as an expression vector, the coding sequences may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing CD26 proteins or peptides in infected hosts.

Specific initiation signals also may be required for efficient translation of CD26 peptides or proteins. These signals include the ATG initiation codon and adjacent sequences. Exogenous translational control signals, including the ATG initiation codon, may additionally need to be provided. One of ordinary skill in the art would readily be capable of determining this and providing the necessary signals. It is well known that the initiation codon must be in-frame (or in-phase) with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators (Bittner et al., 1987).

In eukaryotic expression, one will also typically desire to incorporate into the transcriptional unit an appropriate polyadenylation site (e.g., 5-AATAAA-3) if one was not contained within the original cloned segment. Typically, the poly A addition site is placed about 30 to 2000 nucleotides “downstream” of the termination site of the protein at a position prior to transcription termination.

For long-term, high-yield production of recombinant CD26 peptides or proteins stable expression is preferred. For example, cell lines that stably express constructs encoding CD26 peptides or proteins may be engineered. Rather than using expression vectors that contain viral origins of replication, host cells can be transformed with vectors controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines.

A number of selection systems may be used, including, but not limited, to the herpes simplex virus thymidine kinase (Wigler et al., 1977), hypoxanthine-guanine phosphoribosyltransferase (Szybalska et al., 1962) and adenine phosphoribosyltransferase genes (Lowy et al., 1980), in tk-, hgprt- or aprt-cells, respectively. Also, antimetabolite resistance can be used as the basis of selection for dhfr, that confers resistance to methotrexate (Wigler et al., 1980; O'Hare et al., 1981); gpt, that confers resistance to mycophenolic acid (Mulligan et al. 1981); neo, that confers resistance to the aminoglycoside G418 (Colberre-Garapin et al., 1981); and hygro, that confers resistance to hygromycin (Santerre et al., 1984).

(vi) Protein Purification

In addition, as purified CD26 peptides or proteins are also useful to the methods of the invention, the following is a discussion on protein purification techniques. These techniques are well known to those of skill in the art and involve, at one level, the crude fractionation of the cellular milieu to polypeptide and non-polypeptide fractions. Having separated the polypeptide from other proteins, the polypeptide of interest may be ether purified using chromatographic and electrophoretic techniques to achieve partial or complete purification (or purification to homogeneity). Analytical methods particularly suited to the preparation of a pure peptide are ion-exchange chromatography, exclusion chromatography, polyacrylamide gel electrophoresis; isoelectric focusing. A particularly efficient method of purifying peptides is fast protein liquid chromatography or even HPLC.

Certain aspects of the present invention concern the purification, the partial purification, and in particular embodiments, the substantial purification, of the CD26 protein or peptide. The term “purified protein or polypeptide or peptide” or “partially purified protein or polypeptide or peptide” as used herein, is intended to refer to a composition, isolatable from other components, wherein the protein or polypeptide or polypeptide or peptide is purified to any degree relative to its naturally-obtainable state. A purified protein or polypeptide or peptide therefore also refers to a protein or polypeptide or peptide, free from the environment in which it may naturally occur.

Generally, “purified” will refer to a composition comprising a protein or polypeptide or peptide that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition. The term “isolated”, when used to describe the composition disclosed herein, means protein that has been identified and separated and/or recovered from a component of its natural environment. Contaminant components of its natural environment are materials that would interfere with preventive or therapeutic uses for the protein, and may include other proteinaceous or non-proteinaceous solutes. “Essentially pure” protein means a composition comprising at least about 90% by weight of the protein, based on total weight of the composition, preferably at least about 95% by weight. “Essentially homogeneous” protein means a composition comprising at least about 99% by weight of protein, based on total weight of the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “fold purification number.” The actual units used to represent the amount of activity will, of course, be dependent upon the particular assay technique chosen to follow the purification and whether or not the protein or peptide exhibits a detectable activity. In the instant invention the CD26 peptide, or polypeptide can be detected by using antibodies or by its DPPIV enzymatic activity. Such enzymatic assay's and antibodies, such as IF7 and SF8 etc., are described infra in the section called ‘Examples’.

Other techniques for purifying a protein include, for example, precipitation with ammonium sulphate, PEG, antibodies and the like or by heat denaturatibon, followed by centrifugation; chromatography steps such as ion exchange, gel filtration, reverse phase, hydroxylapatite and affinity chromatography; isoelectric focusing, gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide comprising always be provided in their most purified state. Indeed, it is contemplated that less substantially purified products will have utility in certain embodiments. Partial purification may be accomplished by using fewer purification steps in combination, or by utilizing different forms of the same general purification scheme, to obtain a “partially purified protein”. For example, it is appreciated that a cation-exchange column chromatography performed utilizing an HPLC apparatus will generally result in a greater “fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi. et al., 1977). It will therefore be appreciated that under differing electrophoresis conditions, the apparent molecular weights of purified or partially purified expression products may vary.

High Performance Liquid Chromatography (HPLC) and FPLC are characterized by a very rapid separation with extraordinary resolution of peaks. This is achieved by the use of very fine particles and high pressure to maintain an adequate flow rate. Separation can be accomplished in a matter of minutes or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple-matter to molecular weight.

Affinity chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH; ionic strength, temperature, etc.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography.

### G. THERAPEUTIC METHODS

(i) Protein Therapy

One method for preventing and/or treating cancer is the provision, to a subject, of a peptide or polypeptide encoding a CD26 molecule in combination with a chemotherapeutic agent and/or with a radiotherapeutic agent. Another therapeutic method of the present invention is the provision of a CD26 peptide or polypeptide to an individual to potentiate immune responses during conditions such as infections, cancer or immunosuppression. Such polypeptides may encode the entire CD26 molecule or a fragment of CD26. For example, the fragment may encode a catalytically active fragment for the enzyme. The polypeptides further may encode wildtype, isoforms, mutants, fusions, or any biologically functionally equivalent molecule of CD26.

Alternatively, the therapeutic polypeptides described herein can be synthetic peptides, or mimetics or any other analog thereof The polypeptide may be produced by recombinant expression means or, if small enough, generated by an automated peptide synthesizer. The polypeptides may also be substantially or partially purified by methods described supra. Formulations would be selected based on the route of administration and purpose including but not limited to liposomal formulations and classic pharmaceutical preparations.

(ii) Gene Therapy

Another set of therapeutic embodiments contemplated by the present invention is to provide, to a cancer cell, an expression construct that expresses a CD26 polypeptide and/or the DPPIV activity, in that cell, in conjunction with a chemotherapeutic and/or radiotherapeutic agent. Another aspect of the invention is therapeutic methods to potentiate immune responses during conditions such as infections, cancer or immunosuppression by providing to an individual an expression vector encoding a CD26 peptide or polypeptide.

Because the sequence homology between the human, mouse, rat, rabbit, bovine, primate and dog genes, any of the nucleic acids encoded by the equivalent CD26 genes of these animals could be used in human therapy, as could any of the gene sequence variants which would encode the same, or a biologically equivalent polypeptide. The lengthy discussion above of expression vectors and the genetic elements employed therein is incorporated into this section by reference. Particularly preferred expression vectors are viral vectors.

Those of skill in the art are well aware of how to apply gene delivery to in vivo and ex vivo situations. For viral vectors, one generally will prepare a viral vector stock. Depending on the kind of virus and the titer attainable, one will deliver 1 to 100, 10 to 50, 100-1000, or up to 1×10⁴, 1×10⁵, 1×10⁶, 1×10⁷, 1×10⁸, 1×10⁹, 1×10¹⁰, 1×10¹¹, or 1×10¹² infectious particles to the patient. Similar figures may be extrapolated for liposomal or other non-viral formulations by comparing relative uptake efficiencies. Formulation as a pharmaceutically acceptable composition is discussed below.

Various routes of delivery are contemplated. The vector may be delivered systemically or directly at the inflammation site by local and regional approaches. In some embodiments of the present invention, a subject is exposed to a viral vector and the subject is then monitored for expression construct-based toxicity, where such toxicity may include, among other things, causing a condition that is injurious to the subject.

### H. THERAPEUTIC REGIMENS

Cancers that can be treated by the present invention include, but are not limited to, solid cell tumors and cancers that can be treated include those such as: tumors of the brain (glioblastomas, medulloblastoma, astrocytoma, oligodendroglioma, ependymomas), lung, liver, spleen, kidney, lymph node, small intestine, pancreas, colon, stomach, breast, bone, endocrine glands, endometrium, prostate, testicle, thyroid, ovary, skin, head and neck, esophagus and hematological malignancies including: B-cell leukemias, T-cell leukemia, blood cancer, myeloid leukemia, monocytic leukemia, myelocytic leukemia, promyelocytic leukemia, myeloblastic leukemia, acute myelogenous leukemic, chronic myelogenous leukemic, lymphoblastic leukemia, hairy cell leukemia. Furthermore, the cancer may be a precancer, a metastatic and/or a non-metastatic cancer.

The invention provides the treatment of cancers using effective amounts of 1) a CD26 composition and 2) a chemotherapeutic agent and/or a radiotherapeutic agent. “Effective amount” is defined as an amount of each agent that in combination will decrease, reduce, inhibit or otherwise abrogate the growth of a cancer cell, arrest-cell growth, induce apoptosis, inhibit metastasis, induce tumor necrosis, kill cells or induce cytotoxicity in cells.

As two agents are used they may be administered either simultaneously or at different times. For example, one may provide the CD26 composition before the chemotherapeutic agent and/or a radiotherapeutic agent or after therapy with the chemotherapeutic/radiotherapeutic agent. The administration of the chemotherapeutic/radiotherapeutic agent may precede or follow the administration of the CD26 composition by intervals ranging from minutes to days to weeks. In embodiments where the chemotherapeutic/radiotherapeutic agent and the CD26 composition are administered together, one would generally ensure that a significant period of time did not expire between the time of each delivery. In such instances, it is contemplated that one would administer to a patient both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either the chemotherapeutic/radiotherapeutic agent and the CD26 composition will be required to achieve complete cancer therapy. Various combinations may be employed, where the chemotherapeutic/radiotherapeutic agent is “A” and the CD26 composition is “B”, as exemplified below:

| | | | | | | | |
|---|---|---|---|---|---|---|---|
| A/B/A | B/A/B | B/B/A | A/A/B | B/A/A | A/B/B | B/B/B/A | B/B/A/B |
| A/A/B/B | A/B/A/B | A/B/B/A | B/B/A/A | B/A/B/A | B/A/A/B | B/B/B/A | |
| A/A/A/B | B/A/A/A | A/B/A/A | A/A/B/A | A/B/B/B | B/A/B/B | B/B/A/B | |

Other combinations also are contemplated. The exact dosages and regimens of each agent can be suitable altered by those of ordinary skill in the art.

Although only cancer related treatments are described here, this section is also applicable to the treatment of immune conditions and is useful in the methods of the invention that potentiate immune responses during infections, immunosuppressive conditions, cancers etc., by providing a CD26 composition to an antigen presenting cell (APC) or other immune cell. In the combination therapy scheme described above other agents used to treat the immune condition, such as antibiotics, antiviral agents, anti-tumor agent, and/or other immune effectors are represented by “B” and the CD26 formulation is represented by “A.”

Provided below is a description of some other agents and adjunct therapies that are also effective in the treatment of cancer and may be used in combination with the methods of the present invention.

### I. AJUCNT CANCER THERAPIES

In order to further enhance the efficacy of the cancer treatment, use of other therapies used to treat cancer are also contemplated in combination with the methods of the invention. Thus, another therapeutic agent such as a surgery, another gene therapeutic agent, another protein/peptide/polypeptide therapeutic agent, an immunotherapeutic agent, etc. may be used. Such agents are well known in the art.

(i) Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

(ii) Immunotherapy

Immunotherapeutics, generally, rely on the use of immune effector cells and molecules to target and destroy cancer cells. The other immune effector may be, for example, an antibody specific for a marker on the surface of a tumor cell. This antibody in itself may serve as an effector of therapy or it may recruit other cells to actually effect cell killing. Such a therapeutic antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T-cells and NK cells.

In one aspect the immunotherapy can be used to target a tumor cell. Many tumor markers exist and any of these may be suitable for targeting in the context of the present invention. Common tumor markers include carcinoembryonic antigen, prostate specific-antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and , p155. Alternate immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with the CD26-based therapy of this invention will enhance anti-tumor effects.

In yet other aspects, an immunotherapeutic antibody or a targeting antibody maybe conjugated to a CD26 formulation (peptide/protein or expression vector endoding CD26), and be also conjugated to a radiotherapeutic agent or another anticancer agent.

(a) Passive Immunotherapy

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

(b) Active Immunotherapy

In active immunotherapy, an antigenic peptide, polypeptide or protein, or an autologous or allogenic tumor cell composition or “vaccine” is administered, generally with a distinct bacterial adjuvant (Ravindranath & Morton, 1991; Morton et al., 1993).

(c) Adoptive Immunotherapy

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989). To achieve this, one would administer to an animal, or human patient, an immunologically effective amount of activated lymphocytes in combination with an adjuvant-incorporated antigenic peptide composition as described herein. The activated lymphocytes will most preferably be the patient's own cells that were earlier isolated from a blood or tumor sample and activated (or “expanded”) in vitro.

(iii) Gene Therapy

In yet another embodiment, gene therapy in conjunction with the CD26based therapy described in the invention is contemplated. A variety of nucleic acids and proteins encoded by nucleic acids are encompassed within the invention, some of which are described below. Table 4 lists various genes that may be targeted for gene therapy of some form in combination with the present invention.

TABLE 4

| | | | |
|---|---|---|---|
| Gene | Source | Human Disease | Function |

Growth Factors

| | | |
|---|---|---|
| HST/KS | Transfection | FGF family member |
| INT-2 | MMTV promoter | FGF family member |
| | Insertion | |
| INTI/WNTI | MMTV promoter | Factor-like |
| | Insertion | |
| SIS | Simian sarcoma virus | PDGF B |

Receptor Tyrosine Kinases

| | | | |
|---|---|---|---|
| ERBB/HER | Avian erythroblastosis | Amplified, deleted | EGF/TGF-α/ |
| | virus; ALV promoter | squamous cell | Amphiregulin/ |
| | insertion; amplified | cancer; glioblastoma | Hetacellulin receptor |
| | human tumors | | |
| ERBB-2/NEU/HER-2 | Transfected from rat | Amplified breast, | Regulated by NDF/ |
| | Glioblastomas | ovarian, gastric cancers | Heregulin and EGF- |
| | | | Related factors |
| FMS | SM feline sarcoma virus | | CSF-1 receptor |
| KIT | HZ feline sarcoma virus | | MGF/Steel receptor |
| | | | Hematopoieis |
| TRK | Transfection from | | NGF (nerve growth |
| | human colon cancer | | Factor) receptor |
| MET | Transfection from | | Scatter factor/HGF |
| | human osteosarcoma | | Receptor |
| RET | Translocations and point | Sporadic thyroid cancer; | Orphan receptor Tyr |
| | mutations | familial medullary | Kinase |
| | | thyroid cancer; | |
| | | multiple endocrine | |
| | | neoplasias 2A and 2B | |
| ROS | URII avian sarcoma | | Orphan receptor Tyr |
| | Virus | | Kinase |
| PDGF receptor | Translocation | Chronic | TEL(ETS-like |
| | | Myelomonocytic | transcription factor)/ |
| | | Leukemia | PDGF receptor gene |
| | | | Fusion |
| TGF-β receptor | | Colon carcinoma | |
| | | mismatch mutation | |
| | | target | |

NONRECEPTOR TYROSINE KINASES

| | | | |
|---|---|---|---|
| ABI. | Abelson Mul.V | Chronic myelogenous | Interact with RB, RNA |
| | | leukemia translocation | polymerase, CRK, |
| | | with BCR | CBL |
| FPS/FES | Avian Fujinami SV; GA | | |
| | FeSV | | |
| LCK | Mul.V (murine leukemia | | Src family; T-cell |
| | virus) promoter | | signaling; interacts |
| | insertion | | CD4/CD8 T-cells |
| SRC | Avian Rous sarcoma | | Membrane-associated |
| | Virus | | Tyr kinase with |
| | | | signaling function; |
| | | | activated by receptor |
| | | | kinases |
| YES | Avian Y73 virus | | Src family; signaling |

SER/THR PROTEIN KINASES

| | | |
|---|---|---|
| AKT | AKT8 murine retrovirus | Regulated by PI(3)K?; |
| | | regulate 70-kd S6 k? |
| MOS | Maloney murine SV | GVBD; cystostatic |
| | | factor, MAP kinase |
| | | kinase |
| PIM-1 | Promoter insertion | |
| | Mouse | |
| RAF/MIL | 3611 murine SV; MH2 | Signaling in RAS |
| | avian SV | Pathway |

MISCELLANEOUS CELL SURFACE¹

| | | | |
|---|---|---|---|
| APC | Tumor suppressor | Colon cancer | Interacts with catenins |
| DCC | Tumor suppressor | Colon cancer | CAM domains |
| E-cadherin | Candidate tumor | Breast cancer | Extracellular homotypic |
| | Suppressor | | binding; intracellular |
| | | | interacts with catenins |
| PTC/NBCCS | Tumor suppressor and | Nevoid basal cell cancer | 12 transmembrane |
| | Drosophilia homology | syndrome (Gorline | domain; signals |
| | | syndrome) | through Gli homogue |
| | | | CI to antagonize |
| | | | hedgehog pathway |
| TAN-1 Notch | Translocation | T-ALI. | Signaling? |
| homologue | | | |

MISCELLANEOUS SIGNALING

| | | | |
|---|---|---|---|
| BCL-2 | Translocation | B-cell lymphoma | Apoptosis |
| CBL | Mu Cas NS-1 V | | Tyrosine- |
| | | | Phosphorylated RING |
| | | | finger interact Ab1 |
| CRK | CT1010 ASV | | Adapted SH2/SH3 |
| | | | interact Ab1 |
| DPC4 | Tumor suppressor | Pancreatic cancer | TGF-β-related signaling |
| | | | Pathway |
| MAS | Transfection and | | Possible angiotensin |
| | Tumorigenicity | | Receptor |
| NCK | | | Adaptor SH2/SH3 |

GUANINE NUCLEOTIDE EXCHANGERS AND BINDING PROTEINS

| | | | |
|---|---|---|---|
| BCR | | Translocated with ABL | Exchanger, protein |
| | | in CML | Kinase |
| DBL | Transfection | | Exchanger |
| GSP | | | |
| NF-1 | Hereditary tumor | Tumor suppressor | RAS GAP |
| | Suppressor | neurofibromatosis | |
| OST | Transfection | | Exchanger |
| Harvey-Kirsten, N-RAS | HaRat SV; Ki RaSV; | Point mutations in many | Signal cascade |
| | Balb-MoMuSV; | human tumors | |
| | Transfection | | |
| VAV | Transfection | | S112/S113; exchanger |

NUCLEAR PROTEINS AND TANSCRIPTION FACTORS

| | | | |
|---|---|---|---|
| BRCA1 | Heritable suppressor | Mammary | Localization unsettled |
| | | cancer/ovarian cancer | |
| BRCA2 | Heritable suppressor | Mammary cancer | Function unknown |
| ERBA | Avian erythroblastosis | | thyroid hormone |
| | Virus | | receptor (transcription) |
| ETS | Avian E26 virus | | DNA binding |
| EVII | MuLV promotor | AML | Transcription factor |
| | Insertion | | |
| FOS | FBI/FBR murine | | 1 transcription factor |
| | osteosarcoma viruses | | with c-JUN |
| GLI | Amplified glioma | Glioma | Zinc finger; cubitus |
| | | | interruptus homologue |
| | | | is in hedgehog |
| | | | signaling pathway; |
| | | | inhibitory link PTC |
| | | | and hedgehog |
| HMGI/LIM | Translocation t(3:12) | Lipoma | Gene fusions high |
| | t(12:15) | | mobility group |
| | | | HMGI-C (XT-hook) |
| | | | and transcription factor |
| | | | LIM or acidic domain |
| JUN | ASV-17 | | Transcription factor |
| | | | AP-1 with FOS |
| MLL/VHRX + ELI/MEN | Translocation/fusion | Acute myeloid leukemia | Gene fusion of DNA- |
| | ELL with MLL | | binding and methyl |
| | Trithorax-like gene | | transferase MLL with |
| | | | ELI RNA pol II |
| | | | elongation factor |
| MYB | Avian myeloblastosis | | DNA binding |
| | Virus | | |
| MYC | Avian MC29; | Burkitt's lymphoma | DNA binding with |
| | Translocation B-cell | | MAX partner; cyclin |
| | Lymphomas; promoter | | regulation; interact |
| | Insertion avian | | RB?; regulate |
| | leukosis | | apoptosis? |
| | Virus | | |
| N-MYC | Amplified | Neuroblastoma | |
| L-MYC | | Lung cancer | |
| REL | Avian | | NF-κB family |
| | Retriculoendotheliosis | | transcription factor |
| | Virus | | |
| SKI | Avian SKV770 | | Transcription factor |
| | Retrovirus | | |
| VHL | Heritable suppressor | Von Hippel-Landau | Negative regulator or |
| | | syndrome | elongin; transcriptional |
| | | | elongation complex |
| WT-1 | | Wilm's tumor | Transcription factor |

CELL CYCLE/DNA DAMAGE RESPONSE¹⁰⁻²¹

| | | | |
|---|---|---|---|
| ATM | Hereditary disorder | Ataxia-telangiectasia | Protein/lipid kinase |
| | | | homology; DNA |
| | | | damage response |
| | | | upstream in P53 |
| | | | pathway |
| BCL-2 | Translocation | Follicular lymphoma | Apoptosis |
| FACC | Point mutation | Fanconi's anemia group | |
| | | C (predisposition | |
| | | leukemia | |
| MDA-7 | Fragile site 3p14.2 | Lung carcinoma | Histidine triad-related |
| | | | diadenosine 5′,3″″- |
| | | | tetraphosphate |
| | | | asymmetric hydrolase |
| hMLI/MutL | | HNPCC | Mismatch repair; MutL |
| | | | Homologue |
| hMSH2/MutS | | HNPCC | Mismatch repair; MutS |
| | | | Homologue |
| hPMS1 | | HNPCC | Mismatch repair; MutL |
| | | | Homologue |
| hPMS2 | | HNPCC | Mismatch repair; MutL |
| | | | Homologue |
| INK4/MTS1 | Adjacent INK-4B at | Candidate MTS1 | p16 CDK inhibitor |
| | 9p21; CDK complexes | suppressor and MLM | |
| | | melanoma gene | |
| INK4B/MTS2 | | Candidate suppressor | p15 CDK inhibitor |
| MDM-2 | Amplified | Sarcoma | Negative regulator p53 |
| p53 | Association with SV40 | Mutated >50% human | Transcription factor; |
| | T antigen | tumors, including | checkpoint control; |
| | | hereditary Li-Fraumeni | apoptosis |
| | | syndrome | |
| PRAD1/BCL1 | Translocation with | Parathyroid adenoma; | Cyclin D |
| | Parathyroid hormone | B-CLL | |
| | or IgG | | |
| RB | Hereditary | Retinoblastoma; | Interact cyclin/cdk; |
| | Retinoblastoma; | osteosarcoma; breast | regulate E2F |
| | Association with many | cancer; other sporadic | transcription factor |
| | DNA virus tumor | cancers | |
| | Antigens | | |
| XPA | | xeroderma | Excision repair; photo- |
| | | pigmentosum; skin | product recognition; |
| | | cancer predisposition | zinc finger |

(iv) Other Agents

It is contemplated that other agents may be used in combination with the present invention to improve the therapeutic efficacy of treatment. One form of therapy for use in conjunction with chemotherapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Hormonal therapy may also be used in conjunction with the present-invention. The use of hormones may be employed in the treatment of certain cancers such as breast, prostate, ovarian, or cervical cancer to lower the level or block the effects of certain hormones such as testosterone or estrogen and this often reduces the risk of metastases.

### J. KITS FOR ADMINISTERING CD26 OR VECTORS CODING THEREFOR

The present invention also provides therapeutic kits. In some embodiment, such kits will generally contain, in suitable container means, a pharmaceutically acceptable formulation of a CD26 composition, including a vector or vectors encoding CD26 peptides or polypeptides and/or CD26 proteins or polypeptide formulations, in a form suitable for administration to a subject. The kits may also contain other pharmaceutically acceptable formulations, such as buffers or agents that increase gene uptake or expression or protein uptake.

The kits may have a single container means that contains the protein/peptide or expression construct in a form suitable for administration or the kit may have storage stable forms, along with buffers or diluents in separate and distinct containers. For example, when the components of the kit are provided in one or more liquid solutions, the liquid solution is an aqueous solution, with a sterile aqueous solution being particularly preferred. However, the components of the kit may be provided as dried powder(s). When reagents or components are provided as a dry powder, the powder can be reconstituted by the addition of a suitable solvent. It is envisioned that the solvent may also be provided in another container means.

The container means of the kit may also include at least one device for administration of the CD26 protein/peptide or expression construct. For example, a syringe or inhaler may be included. In some embodiments, the CD26 protein/peptide or expression construct may be pre-mixed and aliquoted into a unit dosage form and loaded into such a device. The kits may contain multiple devices for repeat administration or administration to more than one subject.

The kits of the present invention will also typically include a means for containing the vials, devices or such in close confinement for shipment, storage or commercial sale, such as, e.g., injection or blow-molded plastic containers into which the desired vials and other apparatus are placed and retained. The kits also may contain instructions for administration, including self-administration.

### K. PHARMACEUTICAL COMPOSITIONS

Where clinical applications are contemplated, it will be necessary to prepare pharmaceutical compositions comprising the therapeutic protein(s), and/or therapeutic nucleic acid(s), and/or therapeutic antibodies, and/or expression vectors, and/or virus stocks, and/or drugs, in a form appropriate for the intended application. Generally, this will entail preparing compositions that are essentially free of pyrogens, as well as other impurities that could be harmful to humans or animals.

One will generally desire to employ appropriate salts and buffers. Buffers also will be employed when recombinant cells are introduced into a patient. Aqueous compositions of the present invention comprise an effective amount of the proteins, vectors or drugs to a patient, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the vectors or cells of the present invention, its use in therapeutic compositions is contemplated. Supplementary active ingredients also can be incorporated into the compositions.

Administration of these compositions according to the present invention will be via any common route so long as the target tissue is available via that route. This includes oral, nasal, buccal, rectal, vaginal or topical. Alternatively, administration may be by intradermal, subcutaneous; intramuscular, intraperitoneal or intravenous injection.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, arabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

For oral administration the polypeptides of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The composition may be formulated as a “unit dose.” For example, one unit dose could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### L. ROUTES OF ADMINISTRATION

The routes of administration will vary, naturally, with the location and nature of the cancer or immune cell, and include, e.g., intradermal, intrathecal, intrarthricular, transderimal, parenteral, intravenous, intra-arterial intramuscular, intranasal, subcutaneous, percutaneous, intratracheal, intraperitoneal, intratumoral, perfusion, lavage, direct injection, topical application, and oral administration. Intratumoral injection, or injection into the tumor vasculature is specifically contemplated for discrete, solid, accessible tumors. Local, regional or systemic administration also may be appropriate. In the case of surgical intervention, the present invention may be used before surgery, at the time of surgery, and/or thereafter, to treat residual or metastatic disease. For example, a resected tumor bed may be injected or perfused with a formulation comprising the CD26 formulation and followed by treatment with chemotherapeutic agent. The perfusion may be continued post-resection, for example, by leaving a catheter implanted at the site of the surgery. Periodic post-surgical treatment also is envisioned.

Continuous administration also may be applied where appropriate, for example, where a tumor is excised and the tumor bed is treated to eliminate residual, microscopic disease. Delivery may be via syringe or catherization. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous person will be equivalent to that given by a single or multiple injections, adjusted over a period of time during which the perfusion occurs. It is further contemplated that limb perfusion may be used to administer therapeutic compositions of the present invention, particularly in the treatment of melanomas and sarcomas.

Treatment regimens may vary as well, and often depend on tumor type, tumor location, disease progression, and health and age of the patient. Obviously, certain types of tumors will require more aggressive treatment, while at the same time, certain patients cannot tolerate more taxing protocols. The clinician will be best suited to make such decisions based on the known efficacy and toxicity (if any) of the therapeutic formulations.

In addition to the treatment of cancers, the invention also provides that the CD26 formulations may be used alone or in conjunction with antigens to potentiate the immune system of an individual. In such aspects, antigen-presenting cells are contacted with a CD26 composition. The APC may also optionally be contacted with a tumor or pathogenic antigen or be induced to express a tumor or pathogenic antigen. Alternatively, a human may be administered with the CD26 composition to activate the APCs and optionally also be adminsitered the antigenic composition. The methods and routes of administeratibn described herein apply to these methods as well.

In some embodiments, liposomal formulations comprising CD26 compositions are contemplated. Liposomal encapsulation of pharmaceutical agents prolongs their half-lives when compared to conventional drug delivery systems. Because larger quantities can be protectively packaged, this allow the opportunity for dose-intensity of agents so delivered to cells.

“Liposome” is a generic term encompassing a variety of single and multilamellar lipid vehicles formed by the generation of enclosed lipid bilayers. Phospholipids are used for preparing the liposomes according to the present invention and can carry a net positive charge, a net negative charge or are neutral. Dicetyl phosphate can be employed to confer a negative charge on the liposomes, and stearylamine can be used to confer a positive charge on the liposomes. Liposomes are characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are cationic lipid-nucleic acid complexes, such as lipofectamine-nucleic acid complexes.

### M. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Expression Of CD26 And Its, Associated Dipeptidyl Peptidase IV Enzyme Activity Enhances Sensitivity To Doxorubicin-Induced Cell Cycle Arrest At The G₂/M Checkpoint

Materials and Methods

Cells and Reagents

Human T-cell leukemia Jurkat stable transfectants have been described Dong et al., 1996; Dong et al., 1997; Tanaka et al., 1993). The Jurkat cell lines include: (a) wild-type CD26-transfected Jurkat cell lines (wtCD26); (b) Jurkat cell lines transfected with mutant CD26 containing an alanine at the putative catalytic serine residue at position 630, resulting in a mutant CD26-positive/DPPIV-negative Jurkat transfectant (S630A); (c) Jurkat cell lines transfected with mutant CD26 containing point mutations at ADA-binding site residues 340-343, with amino acids L₃₄₀, V₃₄₁, A₃₄₂, and R₃₄₃ being replaced by amino acids P₃₄₀, S₃₄₁, E₃₄₂, and Q₃₄₃, resulting in a mutant CD26-positive/DPPIV-positive Jurkat transfectant incapable of binding ADA (340-4); (d) vector-only Jurkat transfectant (neo); and (e) nontransfected control Jurkat cells (control). Jurkat transfectants were maintained in culture media, which consisted of RPMI 1640 supplemented with 10% FCS, penicillin (100 units/ml), streptomycin (100 μg/ml), and G418 (0.25 mg/ml; Life Technologies, Inc.). Nontransfected control Jurkat cells were maintained in the same culture media without G418. Jiyoye cells were maintained in the same media but supplemented with 20% FCS, whereas Namalwa cells were maintained in culture supplemented with 7.5% FCS. Anti-p34^{cdc2}, anti-cdc25C, and anti-cyclin B1 were from Santa Cruz Biotechnology (Santa Cruz, Calif.), and anti-actin were from Sigma Chemical Co. Tetrazolium salt MTT (Sigma Chemical Co.) was dissolved at a concentration of 5 mg/ml in sterile PBS at room temperature, with the solution being filter-sterilized and stored at 4° C. in the dark. Extraction buffer was prepared as follows: 20% w/v of SDS was dissolved at 37° C. in a solution of 50% each of N,N-dimethyl fonnamide (Sigma Chemical Co.) and distilled water, pH was adjusted to 4.7 by the addition of 1 M HCl. sDPPIV was produced by Chinese hamster ovary cells as described previously (Ikushima et al., 2000). Doxorubicin was purchased from Calbiochem and was dissolved in sterile PBS.

MTT Assay

Cell growth assay was performed as described previously (Hansen et al., 1989). Cells were incubated in microplates in the presence of culture media alone, culture media and sDPPIV (50 μg/ml), culture media and doxorubicin at the indicated concentrations, or culture media, sDPPIV (50 μl/ml), and doxorubicin at the indicated concentrations, for a total volume of 100 μl (50,000 cells/well). After 48 h of incubation at 37° C., 25 μl of MTT was added to the wells at a final concentration of 1 mg/ml. The microplates were then incubated for 2 h at 37° C., followed by the addition of 100 μl of extraction buffer. After overnight incubation at 37° C., A_{570/nm} measurements at 570 nm were performed, with the SE of the triplicate wells being less than 15%.

Cytotoxicity index was calculated as follows:

$$cytoxicity index ( \% of control ) = 1 - \frac{{A}_{570 / nm} of treated cells}{{A}_{570 / nm} of control cells} \times 100 \%$$

Cell Cycle Analysis

Cells were incubated in culture media alone or culture media and doxorubicin (0.05 μM) at 37° C. for 24 h. Cells were then collected, washed twice with PBS, and resuspended in PBS containing 10 μg/ml propidium iodide, 0.5% Tween 20, and 0.1% RNase at room temperature for 30 min. Samples were then analyzed (FACScan; Becton Dickinson) for DNA content. Cell debris and fixation artifacts were gated out and G₀/G₁, S, and G₂-M populations were quantified using the CellQuest and ModFit LT programs.

SDS-PAGE and Immunoblotting

After incubation at 37° C. in culture media and doxorubicin at the concentrations indicated for 24 h, cells were harvested from wells, washed with PBS, and lysed in lysis buffer, consisting of 1% Brij 97, 5 mM EDTA, 0.02 M HEPES (pH 7.3), 0.15 M NaCl, 1 mM phenylmethylsulfonyl fluoride, 0.5 mM NaF, 10 μg/ml aprotinin, and 0.2 mM sodium orthovanadate. After incubating on ice for 15 min, nuclei were removed by centrifugation and supernatants were collected. Sample buffer (2×) consisting of 20% glycerol, 4.6% SDS, 0.125 M Tris (pH 6.8), and 0.1% bromphenol blue was added to the appropriate aliquots of supernatants. After boiling, protein samples were submitted to SDS-PAGE analysis on an 8% gel under standard conditions using a mini-Protean II system (Bio-Rad). For immunoblotting, the proteins were transferred onto nitrocellulose (Immobilon-P; Millipore). After overnight blocking at 4° C. in blocking solution consisting of 0.1% Tween 20 and 5% BSA in Tris-buffered saline, membranes were blotted with the appropriate primary antibodies diluted in blocking solution for 1 h at room temperature. Membranes were then washed with blocking solution, and appropriate secondary antibodies diluted in blocking solution were then applied for 1 h at room temperature. Secondary antibodies were goat antimouse or goat antribbit horseradish peroxidase conjugates (Dako). Membranes were then washed with blocking solution, and proteins were subsequently detected by chemiluminescence (Amersham Pharmacia Biotech).

Kinase Assays

Cells were treated with doxorubicin at the indicated concentrations for 24 h, and cell extracts were prepared in lysis buffer as described above. Total protein (320 μg) was then diluted to 1 mg/ml protein concentration in HB buffer [20 mM HEPES (pH 7.7), 75 mM NaCl, 2.5 mM EDTA (pH 8.0), 0.05% Triton X-100, 20 mM β-glycerophosphate, 0.5 mM sodium orthovanadate, 1 mM DTT, 2 μg/ml leupeptin, and 1 mM phenylmethylsulfonylfluoride] and immunoprecipitated with anti-p34^{cdc2} monoclonal antibody (Santa Cruz Biotechnology) and protein A-Sepharose beads for 3-4 h. Immunoprecipitates were washed twice with HB buffer and twice with kinase buffer [25 mM HEPES (pH 7.6), 20 mM MgCl₂, and 20 mM β-glycerophosphate]. Kinase assays were performed with cold ATP (50 μM) and [γ-³²P]ATP (5 μCi) in the presence of 1 μg of histone H1 (Life Technologies, Inc.) for 30 min at room temperature, and the reactions were stopped by boiling in Laemmli buffer. Samples were submitted to SDS-PAGE analysis on 12% gel under standard conditions, and the bands were visualized by autoradiography.

Detection of DPPIV Enzyme Activity

DPPIV enzyme activity was detected by using an Enzyme Overlay Membrane system (Enzyme Systems Products, Dublin, Calif.) to which the substrate Ala-Pro-7-amino-4-trifluoromethyl coumarin has been coupled, as described previously (Torimoto et al., 1992) After incubation at 37° C. with media alone or doxorubicin for 24 h, cell lysates were prepared, and sample buffer consisting of 20% glycerol, 4.6%: SDS, 0.125 M Tris (pH 6.8), 0.1% bromphenol blue, and 2% 2-mercaptoethanol was added at room temperature. Samples were then submitted to SDS-PAGE analysis on a 8% gel under standard conditions. The Enzyme Overlay Membrane was moistened in 0.5 M Tris-HCl (pH 7.8), placed over the surface of the gel, and incubated at 37° C. for 40 min in a humidified atmosphere. The membrane was then removed from the gel and placed atop a long-wavelength UV lamp box to monitor enzymatic reaction, which involves the removal of the dipeptide Ala-Pro from the fluorogenic 7-amino-4-trifluoromethyl coumarin and results in the appearance of fluorescent bands on the membrane.

Immunoprecipitation Analysis

Immunoprecipitation analysis was performed as described previously (Dong et al., 1996; Kameoka et al., 1993.) Briefly, Jurkat transfectants or control nontransfected Jurkat cells were labeled by lactoperoxidase-catalyzed iodination. The lysates were incubated with 2 μg of anti-CD26 antibody (1F7) before precipitation with goat antimouse coupled to Affi-Gel (Bio-Rad). Immune complexes were then washed extensively, and eluted by boiling for 5 min in 50 μl of SDS sample buffer before analysis by SDS-PAGE.

Results

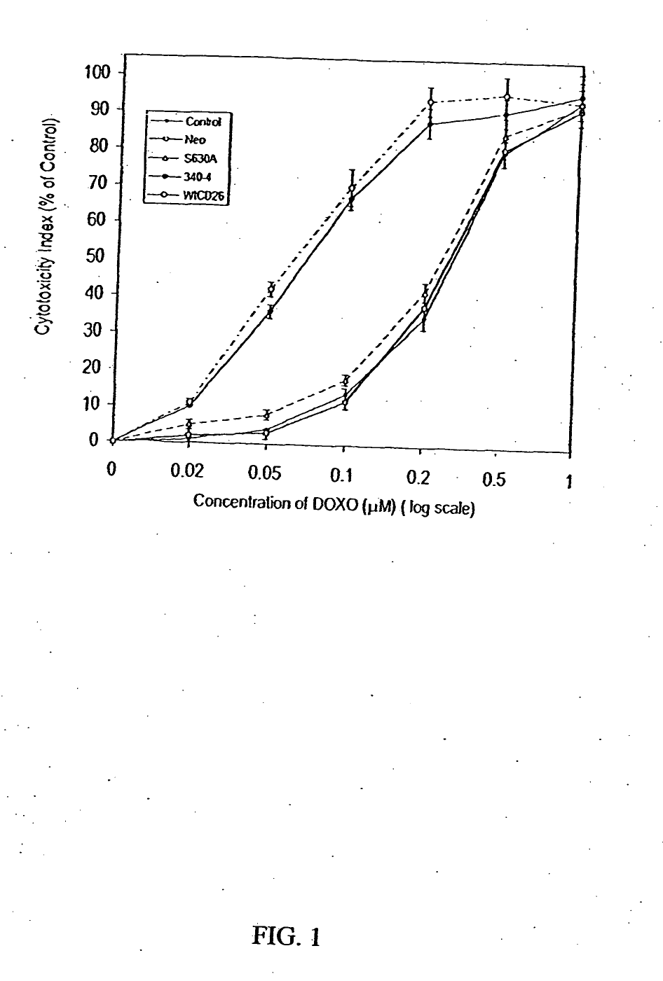

Effect of CD26/DPPIV Expression on Doxorubicin-mediated Growth Inhibition and Cell Cycle Arrest at the G₂-M Checkpoint of Jurkat Cells. Using stable Jurkat transfectants, the effect of CD26 expression on susceptibility to doxorubicin, was investigated by MTT assays. FIG. 1 shows that wild-type CD26 transfectants (wtCD26) displayed markedly increased sensitivity to doxorubicin as compared with parental (control) or vector only (neo) Jurkat cells. CD26 transfectants mutated at the DPPIV catalytic site (S630A) were less sensitive to doxorubicin. On the other hand, Jurkat cells transfected with CD26 mutated at the ADA-binding site while still retaining DPPIV activity (340-4) exhibited greater doxorubicin sensitivity, similar to the wtCD26 transfectants. These data indicate that the presence of CD26, particularly its associated DPPIV enzymatic activity, resulted in enhanced sensitivity to DNA damage mediated by the antineoplastic agent doxorubicin.

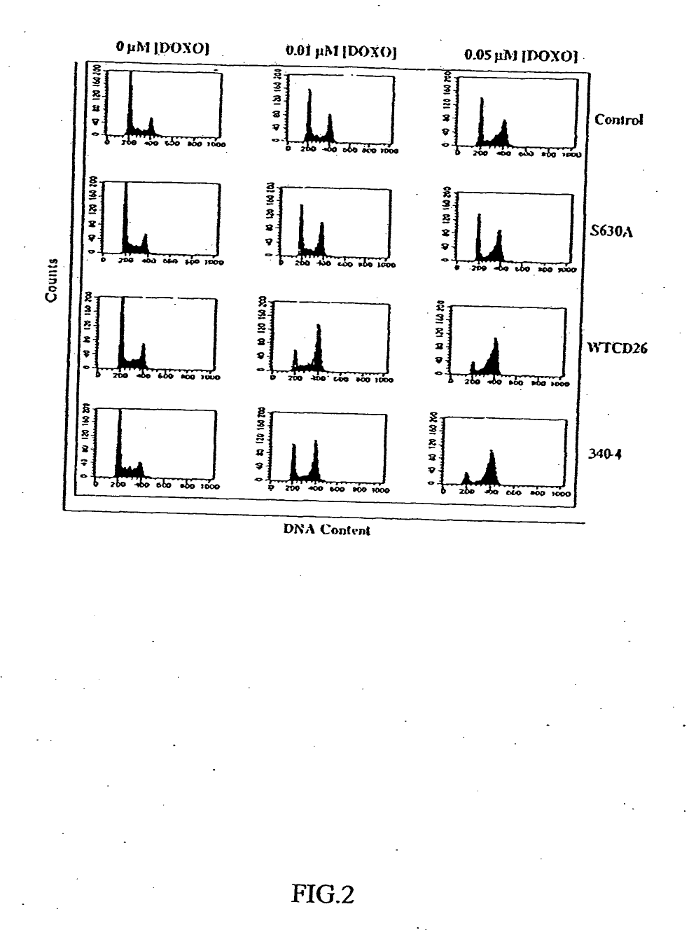

Cell cycle analysis by propidium iodide staining demonstrated that the enhancement in doxorubicin sensitivity seen in wtCD26 transfectants was attributable to an increase in the percentage of cells arresting at G₂-M (FIG. 2; Table 5). Once again, DPPIV enzymatic activity was required for the increased sensitivity to doxorubicin because the G₂-M block in the S630A CD26 mutant was indistinguishable from control parental Jurkat cells, whereas wtCD26 and 340-4 cells exhibited enhanced G₂-M arrest after doxorubicin treatment. Meanwhile, expression of CD26 and its mutant derivatives in the transfected cells were confirmed by immunoprecipitation analysis as well as by immunofluorescence studies, using cells surface-labeled with ¹²⁵I and immunoprecipitated with anti-CD26 monoclonal antibody, as has been described previously (Dong et al., 1996; Dong et al., 1997).

TABLE 5
Enchanced doxorubicin-induced G₂/M arrest in association with
CD26/DPPIV expression^{a}

| | Percentage | Percentage | Percentage |
|---|---|---|---|
| | G₀G₁ | S | G₂/M |
| Media alone | | | |
| Control | 53 | 33 | 14 |
| S630A | 50 | 40 | 10 |
| WtCD26 | 46 | 42 | 12 |
| 340-4 | 49 | 41 | 10 |
| Doxorubicin (0.01 μM) | | | |
| Control | 40 | 26 | 34 |
| S63A | 33 | 32 | 35 |
| WtCD26 | 11 | 27 | 62 |
| 340-4 | 25 | 25 | 50 |
| Doxorubicin (0.05 μM) | | | |
| Control | 32 | 31 | 37 |
| S630A | 27 | 35 | 38 |
| WtCD26 | 6 | 25 | 69 |
| 340-4 | 11 | 11 | 78 |

^{a}Numerical data from FIG 2. CD26 Jurkat transfectants were incubated at 37° C. in media containing doxorubicin for 24 h. Cells wee then harvested, and cell cycle analyses were performed as described in “Materials and Methods.” Data are representative of three separate studies.

DPPIV Enzyme Activity on Jurkat Transfectants. Next, the inventors determined the DPPIV enzyme activity on Jurkat transfectants after treatment with doxorubicin. Jurkat cells were incubated for 24 h at 37° C. with media alone or doxorubicin at 0.01 μM or 0.1 μM. Cells were then harvested, and DPPIV enzyme activity assays were performe. wtCD26 transfectants retained DPPIV enzyme activity after incubation with doxorubicin, whereas S630A transfectants as well as control nontransfected Jurkat cells did not exhibit DPPIV enzyme activity. Therefore, these data indicated that the observed differences in doxorubicin sensitivity in these various Jurkat transfectants were associated with differences in DPPIV enzymatic activity.

Effect of CD26/DPPIV Expression on p34^{cdc2}, Cyclin B1, and cdc25C after Doxorubicin Treatment of Jurkat Cells. p34^{cdc2} is the key regulator of cell cycle progression through G₂-M (King et al., 1994). Treatment with doxorubicin decreased p34^{cdc2} kinase activity, which was associated with cell cycle arrest at the G₂-M checkpoint (Ling et al., 1996; Siu et al., 1999). The activity of p34^{cdc2} kinase was evaluated after doxorubicin treatment in Jurkat cells. For this, Jurkat cells were incubated for 24 h at 37° C. with media containing doxorubicin at the indicated doses. Cells were then harvested, and kinase assays and immunoblotting studies were performed as described above. Lysates were prepared and p34^{cdc2} kinase activity was measured by immunocomplex kinase assay with histone H1 as a substrate. After quantification with phosphoimager, p34^{cdc2} kinase activity of untreated cells was given an arbitrary value of 1, and other activities were measured relative to this value. Protein levels of p34^{cdc2} were examined by immunoblotting studies with anti-p34^{cdc2}. Protein levels of cdc25C were examined by immunoblotting studies with anti-cdc25C. Two major electrophoretic forms reflecting differences in serine-216 phosphorylation were detected. After quantification with phosphoimager, intensity of the cdc25C-P band of untreated cells was given an arbitrary value of 1, and other activities were measured relative to this value. Protein levels of cyclin B1 were examined by immunoblotting studies with anti-cyclin B1 and protein levels of actin were examined by immunoblotting studies with anti-actin. Inhibition of p34^{cdc2} kinase activity occurred at lower concentrations of doxorubicin in wtCD26 Jurkat transfectants as compared with control nontransfectants or S630A transfectants. p34^{cdc2} enzyme activity hence correlated with the observed differences in sensitivity of these Jurkat lines to G₂-M arrest after doxorubicin-induced DNA damage.

Previous studies (Siu et al., 1999; Dunphy, 1994; Poon et al., 1997) have established that hyperphosphorylation of p34^{cdc2} at the inhibitory residues Thr14Tyr15 after doxorubicin-induced DNA damage correlated with inhibition of p34^{cdc2} kinase activity. As assessed by Western blot analysis, doxorubicin-treated wtCD26 Jurkat transfectants had higher levels of hyperphosphorylated p34^{cdc2}, particularly at lower doxorubicin doses as compared with nontransfected Jurkat control cells and S630A transfectants, which only exhibited a slight enhancement in the level of phosphorylated p34^{cdc2} at the higher doses of doxorubicin. the data therefore showed that the relative sensitivity of CD26 Jurkat transfectants to doxorubicin-mediated G₂-M arrest correlated with the relative kinase activity and phosphorylation level p34^{cdc2}, with increasing p34^{cdc2} hyperphosphorylation being associated with decreased p34^{cdc2} kinase activity and enhanced G₂-M arrest.

The cdc25C protein phosphatase is a key regulator of p34^{cdc2} phosphorylation status and kinase activity by dephosphorylating p34^{cdc2} Thr14/Tyr15 residues (Dunphy, 1994; Poon et al., 1997; Nilsson and Hoffman, 2000). Although hyperphosphorylation of cdc25C on NH₂ terminal serine and threonine residues increases its intrinsic phosphatase activity (Lew and Kombluth, 1996), phosphorylation on residue serine-216 results in 14-3-3 protein binding and negatively regulates cdc25C phosphatase activity by preventing cdc25C from interacting with p34^{cdc2} (Peng et al., 1997). Asynchronously growing Jurkat cells have been shown to express two major electrophoretic forms of cdc25C, reflecting differences in serine-216 phosphorylation status (Peng et al., 1997). Consistent with previous findings, the inventors also found that lysates from parental Jurkat cells contained these two major electrophoretic forms of cdc25C. Lysates from wild-type CD26 Jurkat transfectants and S630A transfectants also contained the two forms of cdc25C differing in serine-216 phosphorylation. Importantly, wtCD26 transfectants consistently showed a detectable enhancement in cdc25C serine-216 phosphorylation as compared with S630A transfectants and nontransfected control cells when treated with doxorubicin. These results hence indicated that the increase in cdc25C serine-216 phosphorylation was concordant with enhanced- p34^{cdc2} phosphorylation and concomitant decrease in its kinase activity in doxorubicin-treated wtCD26 Jurkat transfectants as compared with nontransfectants or S630A transfectants.

Next, the inventors investigated the effect of doxorubicin treatment on cyclin B1 expression in the various CD26 Jurkat transfectants, in view of the fact that the p34^{cdc2}/cyclin B1 complex plays a key role in regulating cell cycle progression at the G₂-M checkpoint. Levels of cyclin B1 were higher in wtCD26 Jurkat transfectants as compared with those in nontransfectants or S630A transfectants after treatment with doxorubicin. In some experiments, the inventors did note that the level of cyclin B1 in untreated wtCD26 transfectants were slightly higher than that seen with untreated nontransfectants or S630A transfectants. Significantly, in all of the cases tested, the relative increase in cyclin B1 level in cells treated with doxorubicin as compared with untreated cells was greater in wtCD26 transfectants than it was in parental cells or S630A transfectants. Consistent with previous research that showed that doxorubicin treatment led to cyclin B1 accumulation in murine leukemia P388 cells (Ling et al., 1996), the data thus indicated that doxorubicin treatment in Jurkat cells resulted in alterations in the regulation of cyclin B1 expression, resulting in cyclin B1 accumulation. Importantly, as was the case with p34^{cdc2} phosphorylation status and kinase activity as well as cdc25C serine-216 phosphorylation, the inventors found that the relative level of cyclin B1 expression correlated with the relative sensitivity of CD26 Jurkat transfectants to doxorubicin.

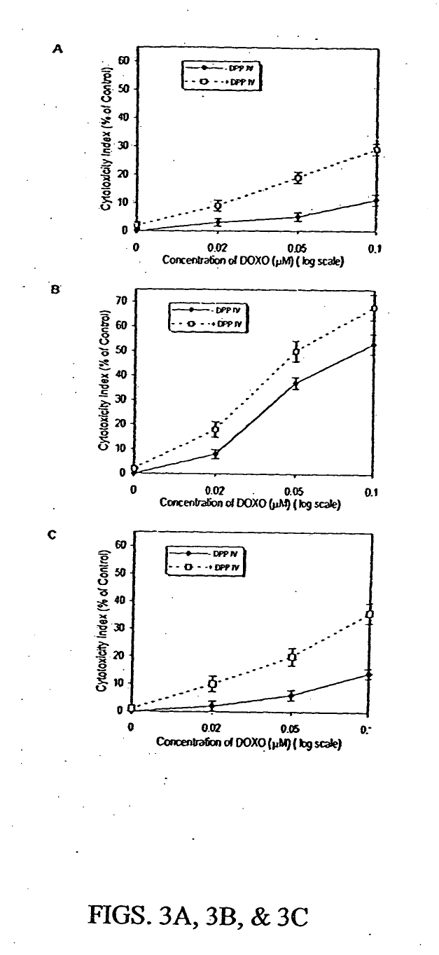

Enhancement of Sensitivity of Jurkat Cells to Doxorubicin by Exogenous sDPPIV. Because the data indicated that the presence of DPPIV enzymatic activity was critical to doxorubicin sensitivity, the inventors next examined the effect of exogenous sDPPIV on doxorubicin-treated CD26 Jurkat transfectants. For these studies, low doses of doxorubicin were used to optimally detect the potential effect of exogenous sDPPIV on doxorubicin sensitivity. Although sDPPIV by itself did not affect MTT uptake in cells incubated in medium alone, the inventors found that the presence of exogenous sDPPIV resulted in significantly enhanced sensitivity to doxorubicin (FIGS. 3A, 3B, & 3C). These data further supported the conclusion that DPPIV enzymatic activity plays a key role in the relative sensitivity of Jurkat cells to doxorubicin.

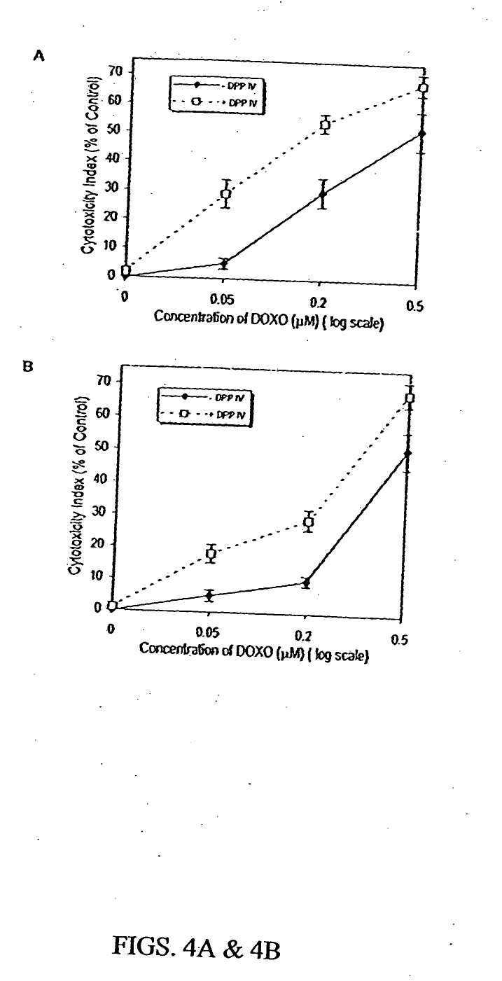

Enhancement of Sensitivity of Cell Lines to Doxorubicin by Exogenous sDPPIV. In view of the fact that the presence of DPPIV activity led to enhanced sensitivity of Jurkat cells to doxorubicin, the inventors examined the effect of sDPPIV on doxorubicin-mediated growth inhibition of other lymphoid cell lines. As seen in FIGS. 4A & 4B, the addition of sDPPIV also enhanced the growth inhibitory effect of doxorubicin in the B-cell lines Namalwa and Jiyoye. The data thus demonstrated that DPPIV-mediated enhancement in sensitivity to doxorubicin was not restricted to Jurkat cell lines alone but was applicable to other lymphoid cell lines.

### Example 2

### Effect Of CD26/Dipeptidyl Peptidase IV On Expression Of Topoisomerase-II Alpha And Cellular Sensitivity To Topoisomerase-II Inhibitors

Materials And Methods

Cells and Reagents. Human T cell leukemia Jurkat stable transfectants have been described (Tanaka et al., 1993; Aytac et al., 2001). The Jurkat cell lines include: 1) wild type CD26-transfected Jurkat cell lines (wtCD26); 2) Jurkat cell lines transfected with mutant CD26 containing an alanine at the putative catalytic serine residue at position 630, resulting in a mutant CD26-positive/DPPIV-negative Jurkat transfectant (S630A); and 3) nontransfected parental Jurkat cells (parental). Jurkat transfectants were maintained in culture media, which consisted of RPMI 1640 supplemented with 10% FCS, penicillin (100 units/ml), streptomycin (100 μg/ml), and G418 (0.25 mg/ml) (Gibco BRL). Nontransfected parental Jurkat cells were maintained in the same culture media without G418. For certain experiments, AIM V serum free media (Gibco) was used rather than culture media containing RPMI 1640 and 10% FCS. In experiments involving AIM V serum-free media, cells were washed extensively with sterile PBS, and then pre-incubated for 24 h at 37° C. with AIM V-containing culture media to prevent contamination with serum. Following pre-incubation, cells were then washed with sterile PBS, and then further incubate in AIM V-containing culture media with or without chemotherapeutic agents at the indicated doses. Anti-p34^{cdc2}, anti-cdc25C, anti-cyclin B1 were from Santa Cruz (Calif.), anti-topoisomerase II alpha was from (Boehringer Mannheim), anti-actin was from Sigma, and the anti-CD26 antibody 1F7 was a murine antibody that has been described previously (Morimoto et al., 1989). Tetazolium salt MTT (3,(4,5-diimethylthiazol-2-yl)2,5-diphenyltetrazolium bromide) (Sigma) was dissolved at a concentration of 5 mg/ml in sterile PBS at room temperature, with the solution being filter sterilized and stored at 4° C. in the dark. Extraction buffer was prepared as follows: 20% w/v of SDS was dissolved at 37° C. in a solution of 50% each of N,N-dimethyl formamide (DMF) (Sigma) and distilled water, pH was adjusted to 4.7 by the addition of 1M HCl. Etoposide and doxorubicin were purchased from Calbiochem and was dissolved in sterile PBS.

MTT Assay. Cell growth assay was performed as described previously (Aytac et al., 2001). Cells were incubated in microplates in the presence of culture media alone, culture media and doxorubicin or etoposide at the indicated concentrations for a total volume of 100 ul (50,000 cells/well). Following 36-48 h of incubation at 37° C., 25 ul of MET was added to the wells at a final concentration of 1 mg/ml. The microplates were then incubated for 2 h at 37° C., followed by the addition of 100 ul of extraction buffer. Following overnight incubation at 37° C., OD measurements at 570 nm were performed, with the standard errors of the mean of the triplicate wells being less than 15%. Cytotoxicity index was calculated as follows:

$$cytoxicity index ( \% of control ) = 1 - \frac{{A}_{570 / nm} of treated cells}{{A}_{570 / nm} of control cells} \times 100 \%$$

Cell Cycle Analysis. Cells were incubated in culture media alone, culture media and doxorubicin or etoposide at indicated concentrations at 37° C. for 24 h. Cells were then collected, washed twice with PBS and resuspended in PBS containing 10 ug/ml propidium iodide, 0.5% Tween-20 and 0.1% RNase at room temperature for 30 min. Samples were then analysed (FACScan, Becton Dickinson) for DNA content. Cell debris and fixation artifacts were gated out and Go-G1, S, and G2-M populations were quantified using the CellQuest and ModFit LT programs.

SDS-PAGE and Immunoblotting. After incubation at 37° C. in culture media and doxorubicin or etoposide at the concentrations indicated for 24 h, cells were harvested from wells, washed with PBS and lysed in lysis buffer, consisting of 1% Brij 97, 5 mM EDTA, 0.02 M HEPES pH 7.3, 0.15 M NaCl, 1 mM PMSF, 0.5 mM NaF, 10 ug/ml aprotinin, and 0.2 mM sodium orthovanadate. After incubating on ice for 15 min, nuclei were removed by centrifugation and supernatants were collected. 2× sample buffer consisting of 20% glycerol, 4.6% SDS, 0.125 M Tris pH 6.8 and 0.1% Bromophenol Blue was added to the appropriate aliquots of supernatants. Following boiling, protein samples were submitted to SDS-PAGE analysis on an 8% gel under standard conditions using a mini-Protean II system (Bio-Rad). For immunoblotting, the proteins were transferred onto nitrocellulose (Immobilon-P, Millipore). Following overnight blocking at 4° C. in blocking solution consisting of 0.1% Tween-20 and 5% bovine serum albumin in TBS, membranes were blotted.with the appropriate primary antibodies diluted in blocking solution for 1 hour at room temperature. Membranes were then washed with blocking solution, and appropriate secondary antibodies diluted in blocking solution were then applied for 1 hour at room temperature. Secondary antibodies were goat anti-mouse or goat anti-rabbit HRP conjugates (Dako, Calif.). Membranes were then washed with blocking solution and proteins were subsequently detected by chemiluminescence (Amersham Pharmacia Biotech).

Detection of DPPIV Enzyme Activity. Dipeptidyl peptidase IV (DPPIV) enzyme activity was detected by using an Enzyme Overlay Membrane system (EOM, Enzyme Systems Products, Dublin, Calif.) to which the substrate Ala-Pro-AFC (7-amino-4-trifluoromethyl Coumarin) has been coupled, as described previously (Aytac et al., 2001). Following incubation at 37° C. with media alone or media with etoposide for 24 h, cell lysates were prepared, and sample buffer consisting of 20% glycerol, 4.6% SDS, 0.125 M Tris pH 6.8, 0.1% Bromophenol Blue and 2% 2-mercaptoethanol was added at room temperature. Samples were then submitted to SDS-PAGE analysis on an 8% gel under standard conditions. The EOM was moistened in 0.5 M Tris HCl, pH 7.8, placed over the surface of the gel, and incubated at 37° C. for 40 min in a humidified atmosphere. The membrane was then removed from the gel and placed atop a long-wavelength ultraviolet lamp box to monitor enzymatic reaction, which involves the removal of the dipeptide Ala-Pro from the fluorogenic AFC and results in the appearance of fluorescent bands on the membrane.

Immunofluorescence. All procedures were carried out at 4° C., and flow cytometry analyses were performed (FACScan, Becton Dickinson) as described previously (Dang et al., 1990). Cells were stained with anti-CD26 antibody and washed two times with PBS and then with goat antimouse IgG FITC (Coulter). Cells were then washed two times with PBS prior to flow cytometry analyses. Negative controls were stained with second antibody alone.

Preparation of Nuclear Extracts for Detection of Topoisomerase II Alpha. For detection of topoisomerase II alpha by immunoblotting, isolation of nuclear fractions from Jurkat cells were prepared as follows. In brief, 10×10⁶ cells were harvested and allowed to swell for 15 min on ice in cytoplasmic extraction buffer (10 mM HEPES, 10 mM KCL, 0.1 mM EDTA, 0.1 mM EGTA, 1 mM DTT, 1 mM PMSF, 2 μg/ml leupeptin, 2 μg/ml Aprotinin, and 0.5 mg/ml Benzamidine). Then NP-40 (final concentration 0.3%) was added into that cell suspension and vortexed for 10 sec. After 1 min centrifugation at 16000×G, the supernatant was removed. The pellet was then incubated with nuclear extraction buffer (20 mM HEPES, 400 mM KCL, 1 mM EDTA, 1 mM EGTA, 1 mM DTT, 0.5 mM PMSF, 2 μg/ml leupeptin, 2 μg/ml Aprotinin, and 0.5 mg/ml Benzamidine) for 30 min on ice with intermittent vortexing. The suspension was centrifuged at 16000×G for 5 min, and supernatant was saved as the nuclear extract. SDS-PAGE and immunoblotting were then performed on the nuclear extracts. Each lane was equally loaded with 15 ug of protein.

Results

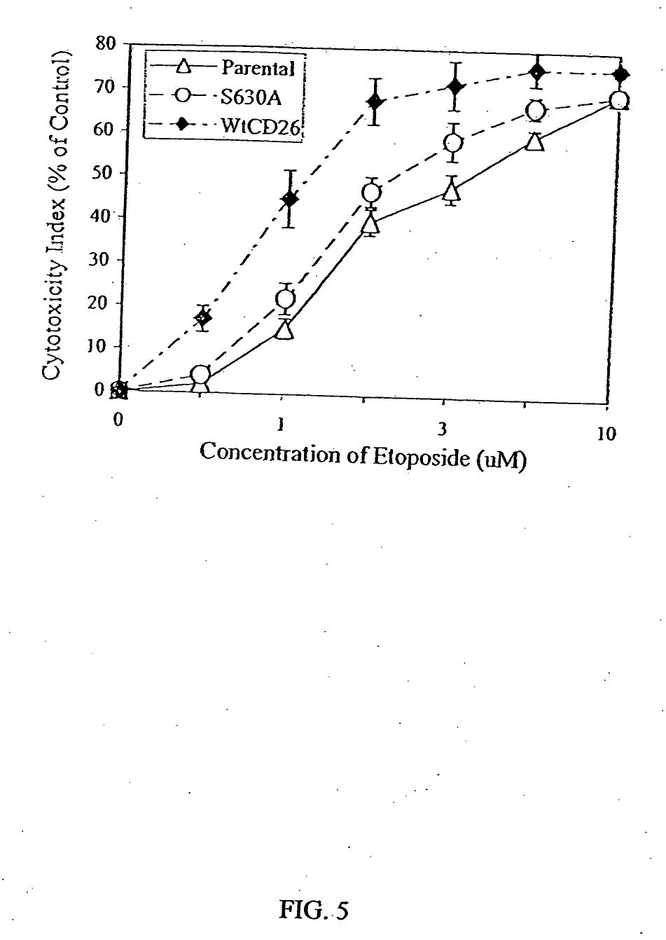

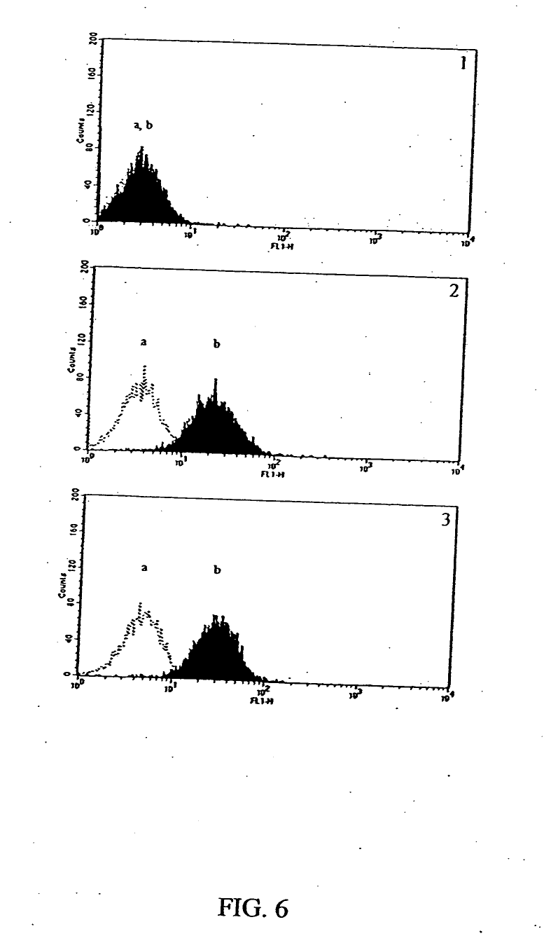

Effect of CD26/DPPIV Expression on Etoposide-Mediated Growth Inhibition and Cell Cycle Arrest at the G₂-M Checkpoint of Jurkat Cells. The effect of CD26/DPPIV on sensitivity to the topoisomerase II etoposide, was examined using stable CD26 transfectants of the Jurkat T leukemia cell line. FIG. 5 shows that wild type CD26 transfectants (wtCD26) displayed significantly increased sensitivity to etoposide compared to parental cells, as monitored by MTT uptake assays. Vector-only Jurkat transfectants also exhibited the same degree of drug sensitivity as parental cells. Significantly, CD26 transfectants mutated at the DPPIV catalytic site (S630A) were also less sensitive to etoposide than wtCD26 transfectants. These data indicate that the presence of CD26 enhances sensitivity to DNA damage mediated by the antineoplastic agent etoposide.

Cell cycle analysis by PI staining demonstrated that the enhancement in etoposide sensitivity seen with wtCD26 transfectants as compared to parental Jurkat cells was due to an increase in the percentage of cells arresting at G₂-M (Table 6). Once again, DPPIV enzymatic activity was required to maximize drug sensitivity to etoposide as the G₂-M block in the S630A CD26 mutant was significantly less than that seen with wtCD26 transfectants.

CD26 Expression and DPPIV Enzyme Activity on Jurkat Transfectants. Data from FIG. 2 showed that CD26 is expressed on the surface of wtCD26 and S630A Jurkat transfectants, and is not expressed on parental Jurkat cells. It was also shown that only wtCD26 Jurkat transfectants expressed DPPIV enzyme activity, which still remains after treatment with etoposide. For this, Jurkat cells were incubated for 24 hours at 37° C. with media alone or etoposide at.0.10 uM or 0.50 uM. Cells were then harvested, and DPPIV enzyme activity assays were performed. On the other hand, S630A transfectants as well as parental Jurkat cells did not exhibit DPPIV enzyme activity. These data indicated that the observed differences in etoposide sensitivity in these various CD26 Jurkat transfectants were associated with differences in DPPIV enzymatic activity.

Effect of CD26/DPPIV Expression on p34^{cdc2} Cyclin B1and cdc25C Following Etoposide Treatment of Jurkat Cells. To elucidate the mechanisms involved in CD26/DPPIV-associated enhancement in etoposide-induced G₂-M arrest, the status of key regulators of this checkpoint were investigated. Jurkat cells were incubated for 24 hours at 37° C. with media containing etoposide at the indicated doses. Cells were then harvested, and immunoblotting studies were performed with appropriate antibodies. It was seen that p34^{cdc2} undergoes hyperphosphorylation at the inhibitory residues Thr14/Tyr15 following etoposide treatment, leading to decreased kinase activity associated with G₂-M arrest (King et al., 1994; Poon et al., 1997; Aytac et al., 2001). As assessed by Western blot analysis, etoposide-treated wtCD26 Jurkat transfectants had higher levels of hyperphosphorylated p34^{cdc2} as compared to parental Jurkat cells and S630A transfectants, which only exhibited a slight enhancement in the level of phosphorylated p₃₄^{cdc2} at the higher doses of etoposide.

The cdc25C protein phosphatase regulates p34^{cdc2} phosphorylation and kinase activity by dephosphorylating p34^{cdc2} Thr14/Tyr15 residues (Poon et al., 1997). While phosphorylation of serine and threonine residues at its amino terminal increases cdc25C intrinsic phosphatase activity (Lew & Kombluth, 1996), phosphorylation on residue serine-216 results in the interaction of 14-3-3 protein with cdc25C to prevent its binding to p34^{cdc2} . (Peng et al., 1997; Dalal et al., 1999), effectively inhibiting cdc25C from dephosphorylating the inhibitory residues of p34^{cdc2}. As demonstrated previously (Peng et al., 1997; Aytac et al., 2001), lysates from asynchronously growing Jurkat cells contained two major electrophoretic forms of cdc25C differing in serine-216 phosphorylation. The studies consistently showed that wtCD26 transfectants displayed increased overall expression of cdc25C and enhancement in cdc25C serine-216 phosphorylation as compared to S630A transfectants and parental cells when treated with etoposide. The increase in cdc25C serine-216 phosphorylation was therefore concordant with enhanced p34^{cdc2} inhibitory phosphorylation in etoposide-treated wtCD26. Jurkat transfectants as compared to parental cells or S630A transfectants.

The studies also showed that the levels of cyclin B1, which associates with p34^{cdc2} as part of the key complex regulating cell cycle progression at the G₂-M checkpoint, were higher in wtCD26 Jurkat transfectants as compared to those in parental cells or S630A transfectants following treatment with etoposide. Cyclin B1 levels were found to be slightly higher in untreated wtCD26 transfectants as compared to untreated parental cells or S630A transfectants (Aytac et al., 2001). Nevertheless, treatment with etoposide consistently resulted in a significant rise in cyclin B1 level in wtCD26 transfectants, an increase greater than that seen in parental Jurkat cells or S630A transfectants. The relative level of cyclin B1 expression therefore correlated with the relative sensitivity of CD26 Jurkat transfectants to etoposide, similar to the case seen with p34^{cdc2} phosphorylation status and cdc25C serine-216 phosphorylation.

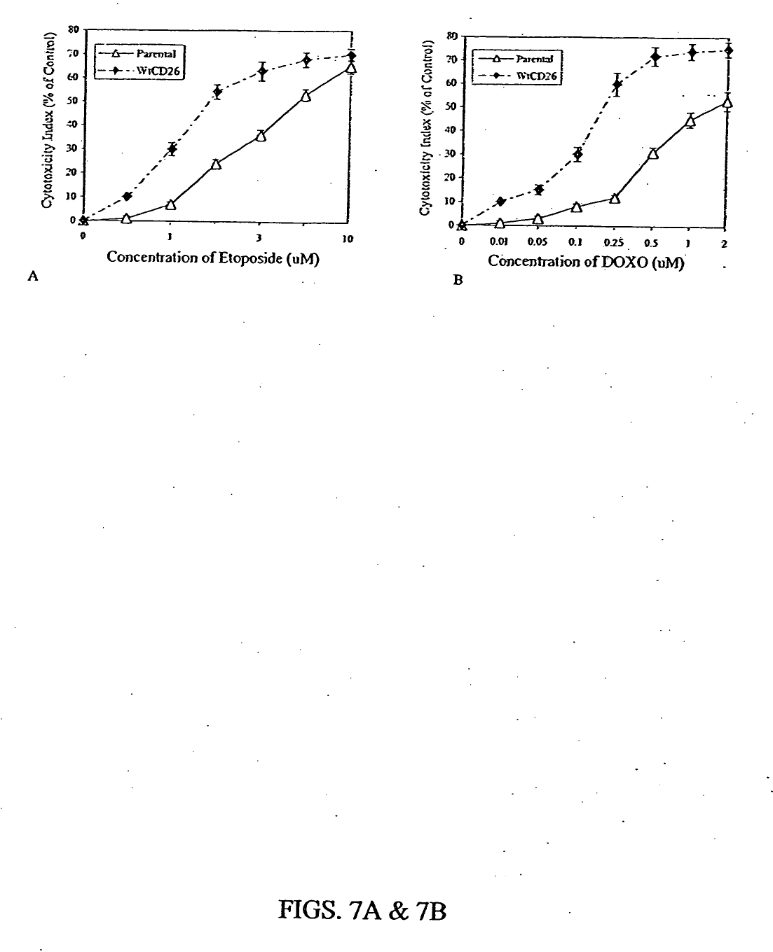

Effect of CD26 Expression on Drug Sensitivity was Independent of Serum. CD26 is able to cleave amino-terminal dipeptides from biological factors with either L-proline or L-alanine at the penultimate position through its DPPIV activity to alter their physiologic functions (Oravecz et al., 1997; Proost et al., 1998). To evaluate the possible role of serum-derived factors in CD26-associated enhancement in drug sensitivity, wtCD26 transfectants or parental Jurkat cells were incubated in AIM V serum-free media, and then performed similar studies as described above following doxorubicin or etoposide treatment. The presence of CD26 resulted in enhanced sensitivity to drug-induced G₂-M arrest in serum-free media, as assessed by MTT uptake studies (FIGS. 7A & 7B) and cell cycle analyses (Table 7). Similarly, wtCD26 Jurkat transfectants exhibited greater p34^{cdc2} phosphorylation, overall expression and serine-216-phosphorylation of cdc25C, and cyclin B1 level when treated with etoposide or doxorubicin as compared to parental Jurkat cells. This effect of CD26 expression on G₂/M checkpoint regulators following drug treatment in serum-free media was studies as as follows: after pre-treatment with AIM V serum-free media at 37° C. for 24 hours, wtCD26 and parental Jurkat cells were incubated at 37° C. in serum-free media containing etoposide or doxorubicin for 24 hours at 37° C. Cells were then harvested and immunoblotting studies were done with appropriate antibodies. These findings hence suggested that the enhanced drug sensitivity associated with CD26/DPPIV expression in CD26 Jurkat transfectants is due to its effect on cell-derived processes rather than due to its interaction with serum-derived factors.

CD26/DPPIV Expression is Associated with Enhanced Topoisomerase II Alpha Level. As antineoplastic agents with key role in the treatment of hematological malignancies, doxorubicin and etoposide both target topoisomerase II alpha. To further explore the potential mechanism of CD26/DPPIV-associated enhancement in drug sensitivity, topoisomerase II alpha expression was examined in CD26 Jurkat transfectants. For this, Jurkat cells were incubated in normal culture media, and nuclear extracts were collected for immunoblotting studies. WtCD26 Jurkat transfectants expressed higher level of topoisomerase II alpha than parental Jurkat cells or S630A transfectants, showing that the increased drug sensitivity in Jurkat cells expressing CD26/DPPIV was associated with enhanced topoisomerase II alpha level.

TABLE 6
Enhanced etoposide-induced G2/M arrest associated with CD26/DPPIV

| | % G₀/G₁ | % S | % G₂/M |
|---|---|---|---|
| Media alone | | | |
| Parental | 49 | 38 | 13 |
| S630A | 47 | 42 | 11 |
| wtCD26 | 46 | 40 | 14 |
| Etoposide (0.10 uM) | | | |
| Parental | 41 | 38 | 21 |
| S630A | 34 | 37 | 29 |
| wtCD26 | 25 | 19 | 56 |
| Etoposide (0.25 uM) | | | |
| Parental | 27 | 32 | 41 |
| S630A | 22 | 30 | 48 |
| wtCD26 | 12 | 19 | 69 |

CD26 Jurkat transfectants and parental cells were incubated at 37° C. in media containing etoposide for 24 h. Cells were then harvested and cell cycle analyses were performed with PI staining. Data are representative of three separate experiments.

TABLE 7
Enhanced sensitivity to etoposide and doxorubicin in serum-free media

| | % G₀/G₁ | % S | % G₂/M |
|---|---|---|---|
| Media alone | | | |
| Parental | 54 | 36 | 10 |
| wtCD26 | 55 | 34 | 11 |
| Etoposide (0.25 uM) | | | |
| Parental | 51 | 30 | 19 |
| wtCD26 | 34 | 23 | 43 |
| Etoposide (0.50 uM) | | | |
| Parental | 36 | 28 | 36 |
| wtCD26 | 18 | 18 | 64 |
| Doxorubicin (0.10 uM) | | | |
| Parental | 43 | 30 | 27 |
| wtCD26 | 31 | 20 | 49 |
| Doxorubicin (0.25 uM) | | | |
| Parental | 36 | 23 | 41 |
| wtCD26 | 14 | 12 | 74 |

Following pre-treatment with AIM V serum-free media at 37° C. for 24 h, wtCD26 Jurkat transfectants and parental cells were incubated at 37° C. in serum-free media with etoposide or doxorubicin for 24 h. Cells were then harvested and cell cycle analyses were performed with PI staining. Data are representative of three separate experiments.

### Example 3

### CD26/Dipeptidyl Peptidase IV Enhances Sensitivity To Apoptosis By Induction of Topoisomerase II Inhibitors

In addition to the finding that CD26/DPPIV expression enhances sensitivity of Jurkat transfectants to G2/M cell cycle arrest induced by topoisomerase II inhibitors and other DNA damaging agent the inventors have also found that CD26/DPPIV enhances sensitivity of the CD26 Jurkat transfectants to apoptosis induced by DNA damaging agents such as doxorubicin and etoposide. In particular, CD26/DPPIV presence is associated with increased susceptibility to the mitochondrial pathway of apoptosis, documented by enhanced cleavage of poly (ADP ribose) polymerase (PARP), caspase-3 and caspase-9, Bcl-xl, and Apaf-1, as well as increased expression of death receptor 5 (DR5). The inventors also show that the caspase-9 specific inhibitor z-LEHD-fmk inhibits etoposide-mediated apoptosis in CD26 Jurkat transfectants, leading to decreased PARP and caspase-3 cleavage, and reduced DR5 expression. Thus, the enhanced cellular sensitivity to topoisomerase II inhibitors in CD26 Jurkat transfectants is due to increased topoisomerase II alpha expression, associated with DPPIV activity. Similarly, addition of soluble CD26 enhances topoisomerase II alpha expression and augments susceptibility to doxorubicin-induced apoptosis. These findings imply a functional interaction between CD26 and topoisomerase II alpha, further evidence of a key role played by CD26 in biological processes.

Topoisomerase II inhibitors are widely used agents in the treatment of solid tumors and hematological malignancies (Froeclich-Ammon and Osheroff, 1995). These drugs selectively exploit the catalytic activity of topoisomerase II alpha by increasing the frequency and duration of DNA cleavage sites, resulting in DNA damage by permanent double stranded breaks (Kellner et al., 2002; Beck et al., 1999). Previous findings have shown that DNA damage mediated by topoisomerase II inhibitors induces apoptosis (Beck et al., 1999; Kaufmann, 1998; Mow et al., 2001), particularly through cytochrome c release (Liu et al., 1996; Kluck et al., 1997; Grad et al., 2001; Yang et al., 1997) and Apaf-1 involvement, which is activated by binding with cytochrome c and dADP (Kuida, 2000; Lauber et al., 2001; Perkins et al., 2000), and then subsequent activation of caspase-9 (Mow et al., 2001). The inventors have shown that surface expression of CD26/DPPIV enhances sensitivity of CD26 Jurkat T-cell transfectants to G2/M arrest mediated by the topoisomerase II inhibitor doxorubicin (see Example 2 and Aytac et al., 2001). In this Example it is demonstrated that surface expression of CD26/DPPIV enhances sensitivity of CD26 Jurkat T-cell transfectants to apoptosis induced by topoisomerase II inhibitors, associated with increased cleavage of Apaf-1, pro-caspase-9, pro-caspase-3,and PARP. Further, CD26 Jurkat transfectants exhibit enhanced Bcl-xl cleavage and increased expression of DR5 following drug treatment. Meanwhile, pre-treatment with the caspase-9 specific inhibitor, z-LEHD-fmk, significantly reduces etoposide-mediated apoptotic events.

Thus, CD26-associated enhancement in sensitivity to topoisomerase II inhibitors in these transfectants is related to enhanced expression of the target enzyme topoisomerase II alpha Further evidence of the association between CD26/DPPIV and topoisomerase II alpha stem from treatment with the DPPIV chemical inhibitor diisopropyl fluorophosphate (DFP) which reduces expression of topoisomerase II alpha in CD26 Jurkat transfectants. The addition of soluble CD26 molecules also enhances topoisomerase II alpha level, with a resultant increase in sensitivity to doxorubicin-mediated apoptosis. These findings emphasize the increasingly important role of the multifaceted CD26/DPPIV molecule in biological processes, while the functional association between CD26/DPPIV and topoisomerase II alpha may be exploited for designing treatments of cancers.

Cells and Reagents. Human:CD26 Jurkat T-cell leukemia stable transfectants have been described and characterized previously regarding CD26 surface expression and associated DPPIV enzyme activity (Dong et al., 1996; Aytac et al. 2001; Dong et al., 1997; Tanaka et al., 1993). The Jurkat cell lines include: (a) wild-type CD26-transfected Jurkat cell lines (wtCD26); (b) Jurkat cell lines transfected with mutant CD26 containing an alanine at the putative catalytic serine residue at position 630, resulting in a mutant CD26-positive/DPPIV-negative Jurkat transfectant (S630A); (c) Jurkat cell lines transfected with mutant CD26 containing point mutations at ADA-binding site residues 340-343, with amino acid L340, V341, A342, and R343 being replaced by amino acids P340, S341, E342 and Q343, resulting in a mutant CD26-positive/DPPIV-positive Jurkat transfectant incapable of binding ADA (340-4); and (d) nontransfected control Jurkat cells (parental). Jurkat transfectants were maintained in culture media, which consisted of RPMI 1640 supplemented with 10% FCS, penicillin (100 units/ml), streptomycin (100 μg/ml), and G418 (0.25 mg/ml; Life Technologies Inc.). Nontransfectant control Jurkat cells were maintained in the same culture media without G418. Annexin V-FITC was from BD PharMingen. Anti-PARP, cytochrome c, and caspase-3 Abs were from BD PharMingen; anti-actin was from Sigma Chemical Co; anti-caspase-9 was from Cayman. Anti-DR5 Abs were from Cayman. Anti-Bcl-xl and Apaf-1 Abs were from BD Transduction Laboratories. Anti-topoisomerase II alpha was from Roche. Caspase-9 inhibitor (z-LEHD-fmk) was from BD PharMingen. Diisopropyl fluorophosphate (DFP) was obtained from SIGMA. Substrate for DPPIV, Gly-Pro-p-nitroanilide-tosylate (GPNT), was purchased from WAKO, Japan. Etoposide was purchased from SIGMA and was dissolved in sterile DMSO. Doxorubicin was purchased from Calbiochem and was dissolved in sterile PBS. Soluble CD26 molecules were produced by Chinese hamster ovary cells and purified as described previously (Tanaka et al., 1994).

Annexin/PI Assays. Exposure of phosphatidylserine residues was quantified by surface annexin V staining as previously described (Vermes et al., 1995; Raynal and Pollard, 1994; Martin et al., 1995)). Briefly, cells were washed in binding buffer (10 mM HEPES, pH 7.4, 2.5 mM CaCl₂, 140 mM NaCl), resuspended in 100 μl and incubated with 0.5 μl/ml annexin V-fluoresencein isothiocyanate (FITC) and 2.5 μg/ml propiodium iodide (PI) for 15 minutes in the dark. Cells were then washed again and resuspended in 400 μl of binding buffer, then flow cytometric analysis (FACScan; Becton Dickinson) was performed. Ten thousand cells were acquired per sample using Cellquest software and data were analyzed with the Paint-a-gate software (Becton Dickinson).

SDS-PAGE and Immunoblotting. After incubation at 37° C. in culture media and etoposide or doxorubicin at the concentrations and duration indicated, cells were harvested from wells, washed with PBS, and lysed in lysis buffer consisting of 1% NP-40, 0.5% deotxycolate, 0.1% SDS, 1 mM phenyhmethylsulfonyl fluoride, 1 mM benzamidine, 10 μg/ml aprotinin, 50 μg/ml leupeptin, 10 μg/ml soybean trypsin inhibitor and 1 μg/ml pepstatin. After incubating on ice for 5 min, nuclei were removed by centrifugation and supernatants were collected as whole cell lysates. Sample buffer (4×) consisting of 20% glycerol, 4.6% SDS, 0.5 M Tris (pH 6.8), 4% β-mercaptoethanol, and 0.2% bromophenol blue was added to the appropriate aliquots of supernatants. After boiling, protein samples were submitted to SDS-PAGE analysis on an 8% gel under standard conditions using a mini-Protean II system (Bio-Rad). For immunoblotting, the proteins were transferred onto nitrocellulobse (Immobilon-P; Millipore). After overnight. blocking at 4° C. in blocking solution consisting of 0.1% Tween 20 and 5% BSA in Tris-buffered saline, membranes were blotted with the appropriate primary antibodies diluted in blocking solution for 1 hour at room temperature. Membranes were then washed with blocking solution, and appropriate secondary antibodies diluted in blocking solution were then applied for 1 hour at room temperature. Secondary antibodies were goat antimouse or goat antirabbit horseradish peroxidase conjugates (Dako). Membranes were then washed with blocking solution, and proteins were subsequently detected by chemiluininescence (Amersham Pharmacia Biotech).

DPPIV Enzyme Activity Assays. As previously described (Kajiyama et al., 2002), DPPIV enzyme activity was measured spectrophotometrically using Gly-P-nitroanilide-tosylate (GPNT), a substrate for DPPIV. 1× PBS-washed whole cell suspension was prepared and 5×10⁵ cells were resuspended in 200 μl of PBS into 96-well plate, then GPNT was added at a final concentration of 0.24 mM. The absorption was measured at 405 nm using microplate spectrophotometer (BIO-TEK Instruments, inc.) twice: just before the addition of the substrate and after 60 min incubation at 37° C. DPPIV enzyme activity was calculated from the increase of absorption between 0 min and 60 min.

Inhibition of DPPIV Enzyme Activity. As described previously (Kajiyama et al., 2002; Koreeda et al., 2001), DFP was used as the DPPIV chemical inhibitor for inhibition assays. To evaluate effect of continuous exposure to DFP, wtCD26 transfectants or parental Jurkat cells were incubated in culture media alone (DFP−), culture media containing 100 μM DFP for 2 hours or for 6 hours (DFP+). A representative sample of cells reflecting each treatment condition was obtained for DPPIV enzyme activity assays or to examine topoisomerase II alpha expression. Alternatively, wtCD26 Jurkat transfectants were incubated in culture media; or in culture media with 100 μM DFP for 4 hours; or they were incubated in culture media with 100 μM DFP for 4 hours, then washed twice in PBS to ensure-removal of DFP followed by incubation in culture media for 2 hours or 8 hours. A representative sample of cells reflecting each treatment condition was obtained for DPPIV enzyme activity assays or to examine topoisomerase II alpha expression. For all treatment conditions, trypan blue uptake assays consistently showed >90% cell viability (data not shown).

Preparation of Cytosol Fractions. As previously described (Haridas et al., 2001; Nishimura et al., 2001), Jurkat cells (4.0×10⁷) were suspended in I ml sucrose buffer (250 mM sucrose in 30 mM Tris HCl, pH 7.4) and transferred into an N₂ cavitation chamber (PARR Instruments, Moline, Ill.). The cells were subjected to N₂ cavitation (250 psi for 5 min) according to the manufacturer's instructions. Under these conditions, most of the cell membrane was disrupted with no change in the mitochondrial respiratory activity. Next, DNA and the nuclear fraction were removed by centrifugation (1,500×g for 2 min). The supernatant was further centrifuged (16,000×g for 10 min), and the supernatant was used as the cytosol fraction.

Preparation of Nuclear Extracts. 10×10⁶ cells were harvested and allowed to swell for 15 minutes on ice in cytoplasmic extraction buffer (10 mM HEPES, 10 mM KCL, 0.1 mM EDTA, 0.1 mM EGTA, 1 mM DTT, 1 mM PMSF, 2 μg/ml leupeptin, 2 μg/ml aprotinin, and 0.5 mg/ml benzamidine). Then NP-40 (final concentration 0.3%) was added into that cell suspension and vortexed for 10 seconds. After 2 minute-centrifugation at 16000×G, the supernatant was discarded. The pellet was then incubated with nuclear extraction buffer (20 mM HEPES, 400 mM KCL, 1 mM EDTA, 1 mM EGTA, 1 mM DTT, 0.5 mM PMSF, 2 μg/ml leupeptin, 2 μg/ml aprotinin, and 0.5 mg/ml benzamidine) for 30 minutes on ice with intermittent vortexing. The suspension was centrifuged at 16000×G for 6 minutes, and supernatant was saved as the nuclear extract.

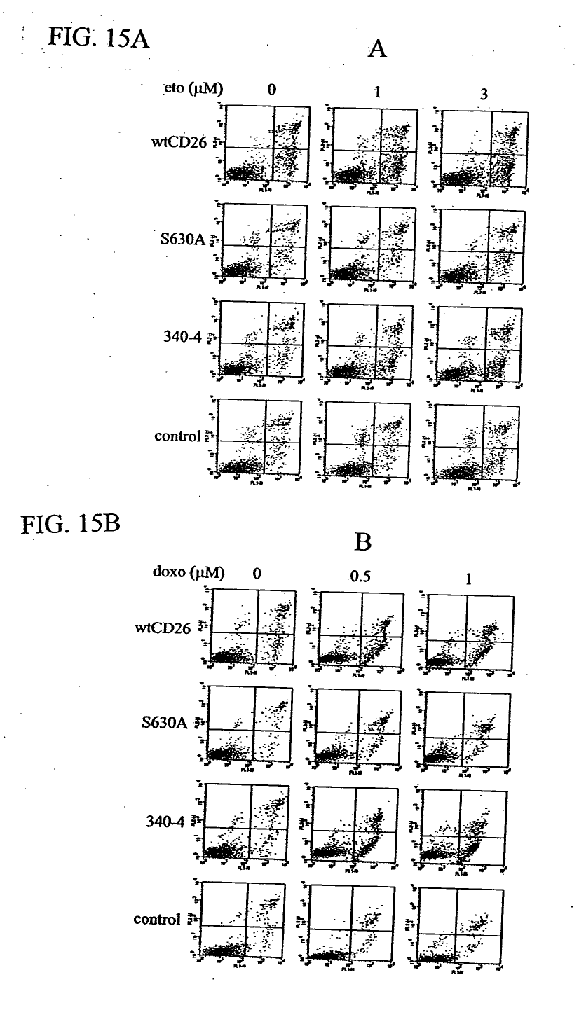

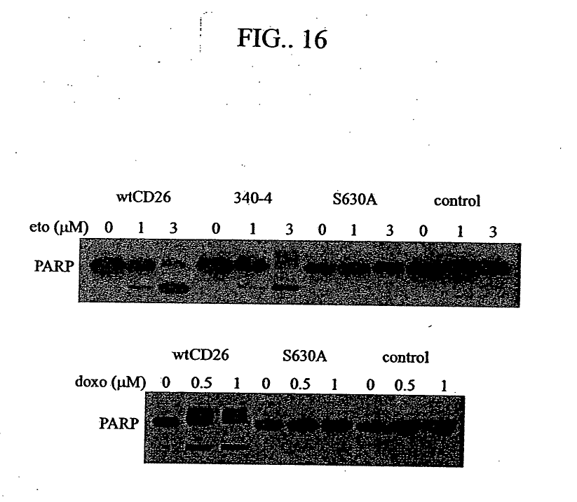

Effect of CD26/DPPIV expression on apoptosis of Jurkat cells mediated by topoisomerase II inhibitors. Using stable Jurkat transfectants, the effect of CD26 expression on susceptibility to etoposide and doxorubicin was investigated. Annexin V/PI assays show that wtCD26. transfectants are more sensitive to the apoptotic effect of etoposide than S630A or parental control Jurkat cells. Meanwhile, 340-4 transfectants (340-4) exhibit higher level of drug-induced apoptosis, similar to that of wtCD26 transfectants (FIG. 15A). Furthermore, wtCD26 and 340-4 cells display greater apoptosis when treated with doxorubicin as compared with parental or S630A Jurkat cells (FIG. 15B). Similarly, both the wtCD26 and 340-4 Jurkat transfectants are more sensitive to etoposide-mediated PARP cleavage (Gao and Dou, 2000; Fulda et al., 2001; Ariumi et al., 1998) (FIG. 16), as compared with parental cells and S630A transfectants. Similar results are seen when cells are treated with doxorubicin. These data indicate that the presence of CD26, especially its associated DPPIV enzymatic activity, enhances apoptosis mediated by topoisomerase II inhibitors.

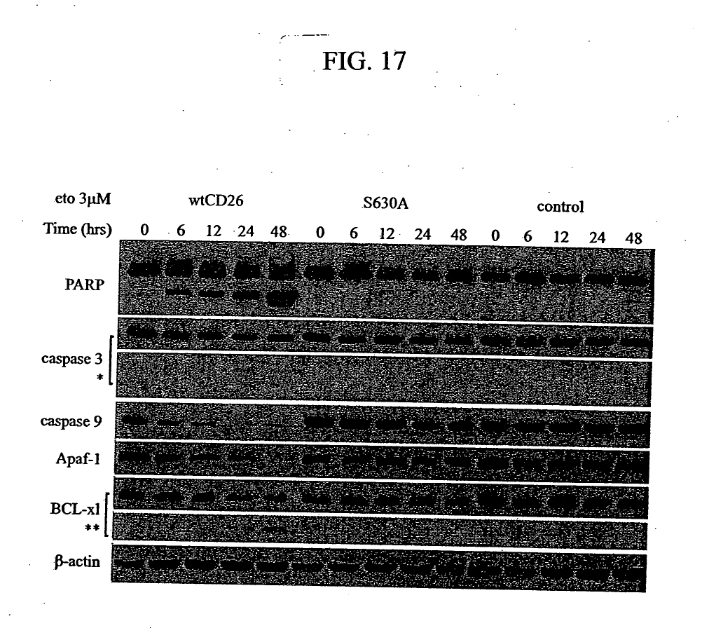

Effect of CD26/DPPIV surface expression on the mitochondrial pathway of apoptosis induced by etoposide. Previous work demonstrated that DNA damage mediated by topoisomerase II inhibitors induces apoptosis through the mitochondrial pathway, leading to subsequent activation of caspase-9 and PARP cleavage (Beck et al., 1999; Kaufmann, 1998; Mow et al., 2001; Liu et al., 1996; Kluck et al., 1997; Grad et al., 2001; Yang et al., 1997; Gao and Dou, 2000a; Gao and Dou, 2000b; Wen et al., 2000). Time-course analyses (FIG. 17) show that etoposide treatment results in enhanced cleavage of PARP and pro-caspase-3, resulting in increased levels of the cleaved 17 kDa caspase-3 bands in wtCD26 transfectants, as compared with S630A and parental Jurkat cells. Moreover, etoposide treatment leads to significantly greater cleavage of pro-caspase-9 in wtCD26 cells compared to S630A and parental control Jurkat. Consistent with previous reports showing that Apaf-1 is a key regulator of the mitochondrial pathway of apoptosis (Kuida, 2000; Lauber et al., 2001; Perkins et al., 2000), it has been demonstrated here that greater cleavage of the 130 kDa proform of Apaf-1 is seen in etoposide-treated wtCD26 Jurkat transfectants as compared-with parental control or S630A Jurkat. Furthermore, the increase in sensitivity to etoposide-induced apoptosis in wtCD26 transfectants is shown herein to be accompanied by greater cleavage of the full-length antiapoptotic molecule Bcl-xl (Fujita et al., 1998; Wang et al., 2001; Basanez et al., 2001) and a resultant rise in the 18 kDa cleaved band. Taken together, these results indicate that CD26/DPPIV enhances etoposide-mediated apoptosis of Jurkat cells by affecting cellular processes known to be involved in drug-mediated apoptosis, including those involving caspase-9 processing and the mitochondrial pathway, as well as processing of bcl-2-related molecules.

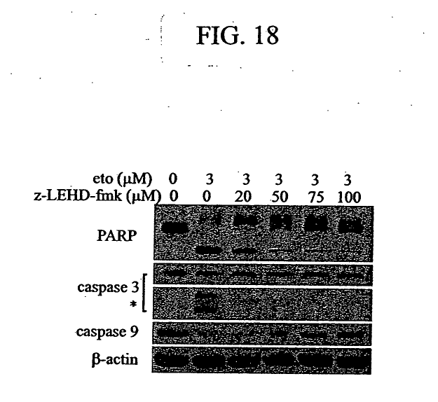

Effect of the caspase-9 inhibitor z-LEHD-fmk on etoposide-induced apoptosis in CD26 Jurkat transfectants. To further confirm the findings that CD26 affects etoposide-induced apoptosis through caspase-9-related events, the present inventors evaluated the effect of the caspase-9 inhibitor z-LEHD-fmk on this process. Western blot analyses show that pre-treatment with z-LEHD-fink, significantly abrogates the effect of etoposide on wtCD26 Jurkat transfectants. As shown in FIG. 18, etoposide-mediated cleavage of pro-caspase-9 is inhibited by z-LEHD-fink in dose-dependent manner. Furthermore, cleavage of pro-caspase-3 and PARP, events downstream of caspase-9 processing, is significantly reduced following pre-treatment with the caspase-9 inhibitor. This data therefore indicate that CD26 augments etoposide-induced apoptosis in CD26 Jurkat transfectants through caspase-9-related events.

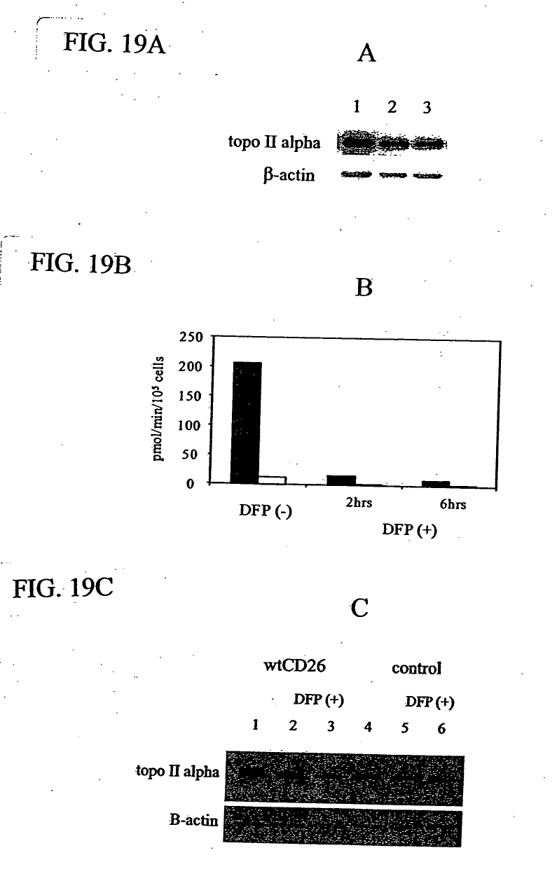

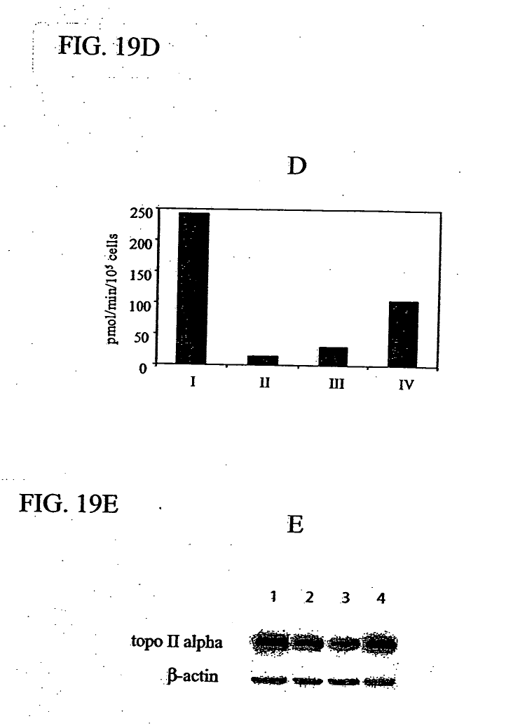

Effect of the DPPIV enzyme inhibitor diisopropyl fluorophosphate on topoisomerase II alpha expression. In view of the effect of CD26, particularly its intrinsic DPPIV enzyme activity, on apoptosis induced by the topoisomerase II inhibitors, topoisomerase II alpha expression in the CD26 Jurkat transfectants was examined. It is demonstrated that topoisomerase II alpha level in wtCD26 Jurkat transfectants is consistently higher than that in S630A transfectants or parental Jurkat (FIG. 19A). In addition, the effect of the DPPIV chemical inhibitor diisopropyl fluorophosphate (DFP) (Kajiyama et al., 2002; Koreeda et al., 2001) on topoisomerase II alpha expression was examined. It is shown that continuous treatment with DFP results in inhibition of DPPIV enzyme activity in wtCD26 Jurkat transfectants (FIG. 19B), associated with decreased expression of topoisomerase II alpha in these cells (FIG. 19C). On the other hand, expression of topoisomerase II alpha in parental Jurkat cells is not significantly affected by continuous exposure to DFP, as expected. Additionally, the status of DPPIV enzyme activity and topoisomerase II alpha expression following DFP treatment was also examined. For this purpose, following treatment with DFP, wtCD26 cells were washed and incubated in culture media for the indicated time periods. It is demonstrated that recovery of DPPIV enzyme activity is associated with recovery of topoisomerase II expression (FIG. 19D and FIG. 19E). These results further corroborate the present inventors earlier findings regarding the importance of DPPIV enzyme activity in topoisomerase II alpha expression in CD26 Jurkat transfectants.

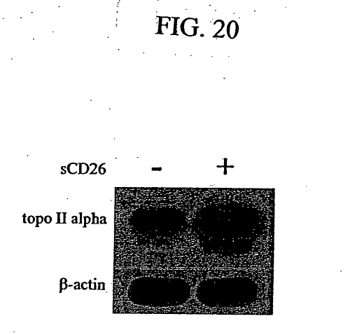

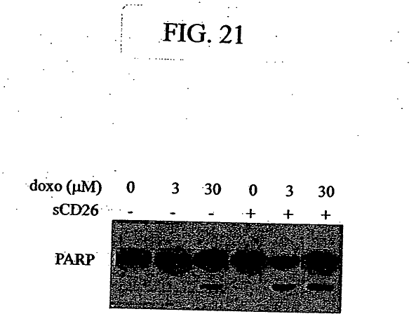

Effect of soluble CD26 molecules on topoisomerase II alpha expression and sensitivity to doxorubicin. To expand on the findings that surface expression of CD26, particularly its intrinsic DPPIV activity, in Jurkat transfectants results in enhanced topoisomerase II alpha level, the effect of soluble CD26 (sCD26) molecules on topoisomerase II alpha expression was examined. As shown in FIG. 20, incubation with sCD26 molecules results in a significant increase in topoisomerase II alpha protein expression in parental control Jurkat cells. Along with an increase in topoisomerase II alpha expression, incubation of parental Jurkat cells with sCD26 molecules also results in enhanced doroxubicin-induced PARP cleavage (FIG. 21). These findings indicate that the presence of CD26/DPPIV augments topoisomerase II alpha expression, leading to a resultant increase in sensitivity to topoisomerase II inhibitors.

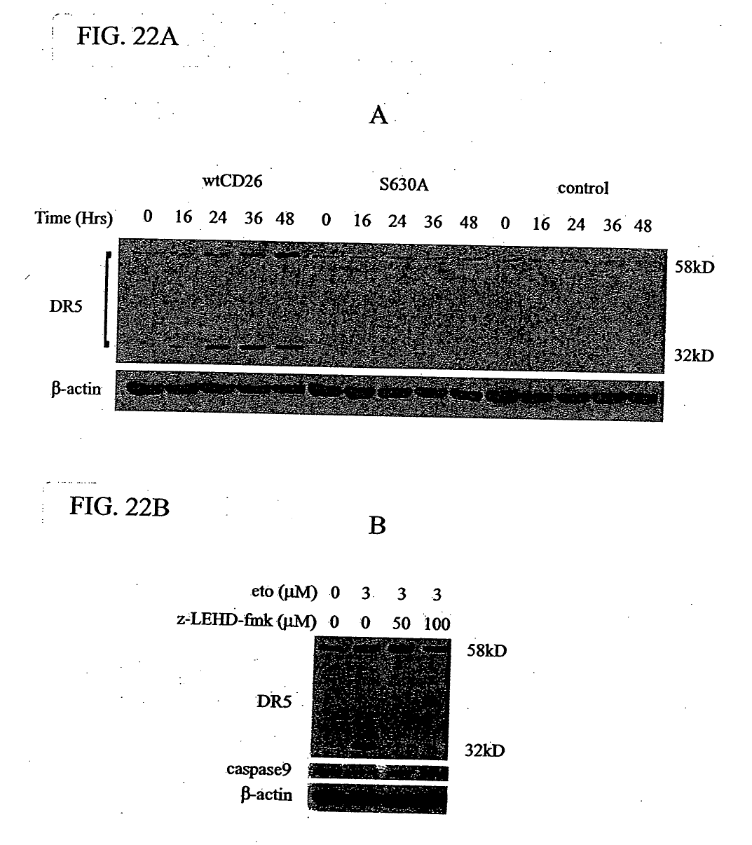

Caspase9-dependent involvement of DR5 in etoposide-induced apoptosis in CD26 Jurkat transfectants. Expression of death receptor 5 (DR5), a member of the TRAIL (tumor necrosis factor-related apoptosis-inducing ligand) family, is upregulated following treatment with such DNA damaging agents as doxorubicin and etoposide (Wen et al., 2000; Nagane et al., 2001; Gibson et al., 2000; Jazirehi et al., 2001). The present inventors demonstrate here that etoposide treatment leads to a greater increase in the levels of the 58 kDa DR5 in wtCD26 transfectants as compared with S630A or parental Jurkat cells (FIG. 22A). Interestingly, Western blotting analyses with anti-DR5 mAb also detect the expression of a smaller 32 kDa band with etoposide treatment, with its levels again being significantly higher in wtCD26Jurkat than S630A or parental cells. In time course studies, the appearance of the 32 kDa band consistently precedes the observed increase in expression levels of the 58 kDa band.

The present inventors have already demonstrated that etoposide-induced apoptosis in wtCD26 Jurkat transfectant involves caspase-9 processing. To determine whether the enhancement in DR5 expression in etoposide-treated wtCD26 Jurkat is dependent on caspase-9-related events, DR5 expression in cells treated with etoposide following pre-incubation with the caspase-9 specific inhibitor z-LEHD-fmk was examiner. As demonstrated in FIG. 22B, the increase in the 58 kDa band seen in etoposide-treated wtCD26 cells is significantly attenuated when cells are pre-incubated with z-LEHD-fink. Similarly, the 32 kDa band induced by etoposide is no longer detectable with z-LEHD-fink pre-incubation.

Thus, in summary it is, shown that the presence of CD26 on Jurkat transfectants, particularly its intrinsic DPPIV enzyme activity, enhances sensitivity to apoptosis induced by the topoisomerase II inhibitors doxorubicin and etoposide. Significantly, this enhanced drug sensitivity is related to increased expression of the target enzyme topoisomerase II alpha Meanwhile, the finding that chemical inhibition of DPPIV activity is associated with reduced topoisomerase II alpha expression, in conjunction with data showing that wtCD26 Jurkat transfectants have higher level of topoisomerase II alpha than the S630A or parental Jurkat cell, provides additional evidence of the key role played by DPPIV in topoisomerase II alpha expression and the resultant sensitivity to topoisomerase II inhibitors. This conclusion is further corroborated by the data showing enhanced topoisomerase II alpha expression and increased drug sensitivity following incubation-of cells with soluble CD26 molecules.

### Example 4

### Soluble CD26/Dipeptidyl Peptidase IV Induces T Cell Proliferation Through CD86 Up-Regulation On APCS

Material and Methods

Isolation and Activation of Human Lymphocyte Populations

Human PBMC, collected from healthy adult volunteers who were immunized with TT within two years before donation, were isolated by centrifugation on Ficoll/Paque (Amersham Pharmacia Biotech, Piscataway, N.J.). PBMC were directly used for the time-course studies. For the reconstitution studies, PBMC were further purified into T cell fractions and APC fractions. To obtain a highly purified T cell population, PBMC were separated into an E rosette-positive (E⁺) population and were used as resting T cells as determined by flow cytometric analysis (FACSCalibur; Nippon BD Biosciences, Tokyo, Japan) using an FITC-labeled anti-CD3 mAb (BD PharMingen, San Diego, Calif.) with purity being >95%. To obtain APC, an E rosette-negative (E⁻) population was adhered to plastic plates for 4 h at 37° C., and adherent cells were used as APC. Monocytes were either purified by a flow cytometer on the basis of PE-labeled anti-CD14 mAb (BD PharMingen)-oriented parameter, or by negative selection through the use of immunomagnetic beads coated with an anti-CD3, CD7, CD19, CD45RA, CD56, and IgE mAb Qailtenyi Biotec, Auburn, Calif.) with purity being >95%. PBMC were cultured in RPMI 1640 medium (Life Technologies, Grand Island, N.Y.) supplemented with 10% FCS, 100 U/ml penicillin, and 100 μg/ml streptomycin (Life Technologies) and TT (Calbiochem, La Jolla, Calif.) at different concentrations for 16 h, followed by sCD26 pulses at various time intervals. T cells were also cultured with the standard medium (10% FCS-RPMI 1640 with penicillin and streptomycin) in the presence or absence of sCD26. Monocytes were incubated with TT at different concentrations for 16 h in the standard medium, followed by the addition of sCD26 in the culture medium at different times. To avoid interference by nonspecific activation of monocytes due to contamination, polymyxin B sulfate (20 IU/ml; Sigma-Aldrich, St. Louis, Mo.) was added to all media and reagents used for APC/monocytes studies.

All cells were preincubated in the standard medium for 24 h to minimize the risk of potential interference from sCD26 present in human serum (Tanaka et al., 1993). In these studies, cells were incubated in 96-well plates (200 μl/well; Falcon, Franklin Lakes, N.J.). For the time-course study, 1.0×10⁵ PBMC were incubated with 0.5 μg/ml TT for 16 h. Then, 0.5 μg/ml sCD26 was added to the well and was incubated for 0, 6, 12, 24, 48, 72, and 96 h. The day when the first sCD26 was added to the culture well was defined as day 0 of culture. All assays in proliferation experiments were performed on day 7 of culture after cells were incubated at 37° C. in a 5% CO₂ humidified atmosphere. For the reconstitution study, 0.5×10⁶ of monocytes were preincubated with TT, at a concentration of 0.5 μg/ml with different concentrations of sCD26 for 24 h. Monocytes were washed with PBS, and then 1×10⁴/well of preincubated monocytes were subjected to the assay with 1×10⁵/well of purified T cells from the same donor as the prepared monocytes. For fixation study, as prefixed stimulation study, after being treated with 0.05% glutaraldehyde for 30 s at room temperature, followed by being washed three times with PBS, 0.5×10⁶ of monocytes were incubated with TT at a concentration of 0.5 μg/ml and with 0.5 μg/ml sCD26. As postfixed stimulation study, 0.5×10⁶ of monocytes were initially incubated with TT at a concentration of 0.5 μg/ml and with 0.5 μg/ml sCD26. Subsequently, the preincubated monocytes were treated with 0.05% glutaraldehyde for 30 s at room temperature, followed by being washed three times with PBS. Monocytes (1.0×10⁴/well) were then subjected to the assay with 1.0×10⁵/well of purified T cells, which originated from the same donor as the prepared monocytes.

Preparation of sCD26

sCD26 with DPPIV (sCD26/DPPIV⁺) was produced according to the method described previously (Manickasingham et al., 1998). Briefly, the expression vector RcSRα-26-days 3-9, which contains a deletion of the coding sequence for amino acids 3-9 of CD26, was transfected into a dihydrofolate reductase-deficit Chinese hamster ovary (CHO) cell line, DXB-11 by electroporation, together with pMT-2 providing the dihydrofolate reductase gene. Mutant sCD26 without DPPIV (sCD26/DPPIV) was produced in the same method except that RcSRα-26d3-9 as further modified to yield RcSRα-26d3-9 S630A, which contains a point mutation at the active site of the DPPIV enzyme (Ser⁶³⁰ was replaced by Ala) by site-directed mutagenesis using the oligonucleotide. The transfected CHO cells, which produce either sCD26 or mutant sCD26, were cultured in serum-free CHO-S-SFM II medium (Life Technologies) supplemented with 1 μM methotrexate (Sigma-Aldrich). The culture supernatant was collected and subjected to affinity chromatography on adenosine deaminase-Sepharose according to the methods described previously (Ikushima et al., 2000).

mAbs and Reagents

The source and working concentration of the mAbs used as primary Abs for flow cytometry are as follows: PE-conjugated anti-CD3 -(UCHT1, mouse IgG1; 10 μg/ml; BD PharMingen), anti-CD14 (Mo-2, mouse IgM; 10 μg/ml; Beckman Coulter, Miami, Fla.), anti-CD19 (HIB19, mouse IgG1; 10 μg/ml; BD PharMingen), and anti-CD56 (NKH1, mouse IgG1; 10 μg/ml; Beckman Coulter); FITC-labeled anti-CD80 (BB1, mouse IgM; 10 μg/ml; BD PharMingen), anti-CD86 (IT2.2, mouse IgG2; 10 μg/ml; BD PharMingen), and anti-HLA-DR (L243, mouse IgG2; 10 μg/ml; BD PharMingen). Oregon green-conjugated sCD26 (sCD26-OG) was made with FluoReporter Oregon green protein labeling kit (Molecular Probes, Eugene, Oreg.) according to the manufacturer's instruction. These Oregon green-conjugated proteins were used at a concentration of 1 μg/ml. Biotinylated anti-CD14 (Mo-2; IgM; 10 μg/ml) was purchased from Beckman Coulter. mAbs for blocking assays were obtained as follows: CD80 (BB-1), CD86 (IT2.2), and HLA-DR. (L243) from were BD PharMingen; the chimeric protein of human CTLA-4 and murine Ig (CTLA-4 Ig) and relevant control murine Ig were purchased from Ancell (Bayport, Minn.); and mouse anti-human M6P/IGFIIR mAb was kindly provided by Dr. V. Horcjsi (Academy of Science of the Czech Republic, Praha, Czech Republic). Texas red-conjugated anti-human M6P/IGF-IIR mAb (M6P/IGF-IIR-red) was made with FluoReporter Texas red protein labeling kit (Molecular Probes) according to the manufacturer's instructions. In all experiments, relevant control-mAbs of the same Ig isotype were included (IgG1 (MOPC-21), IgG2 (G155-178), and IgM (G155-228) were purchased from BD PharMingen). Egg white lysozyme was purchased from Wako Pure Chemical (Osaka, Japan) and was conjugated with Oregon green.

T-Cell Proliferation Assay

T-cell proliferation induced by APC was measured by [³H]TdR (ICN Radiochemicals, Irvine, Calif.) uptake. Seven-day-incubated cells were pulsed with 1 μCi/well of [³H]TdR 8 h before harvesting onto a glass-fiber filter (Wallac, Turk, Finland), and the incorporated radioactivity was quantified by a liquid scintillation counter (Wallac).

Flow Cytometic Analysis

Assessment of the PBMC population that takes up sCD26-OG was performed with PE-conjugated anti-CD3, anti-CD14, anti-CD19, and anti-CD56 mAbs (10 μg/ml). For these experiments, 1×10⁶ PBMC/well were incubated in standard medium containing sCD26-OG for 24 h with or without preincubation with TT. Cells were then washed twice in ice-cold PBS, followed by incubation for 2 min in acidic PBS (pH 3.0) to strip any sCD26-OG attached to the cell surface. Cells were subsequently washed in ice-cold PBS and were incubated with mouse Ig isotypes (1 μg/ml) to block nonspecific binding; this was followed by reaction with PE-conjugated mabs.

In studies assessing the expression of CD80, CD86, and HLA-DR on purified monocytes (0.5×10⁶ monocytes/well), monocytes were preincubated with or without TT (0.5 μg/ml) for 16 h. Following incubation with sCD26 (0.5 μg/ml) for 24 h, FITC-conjugated CD80, CD86, and HLA-DR mAbs (10 μg/ml) were used with PE-conjugated anti-CD14 (10 μg/ml) to gate exclusively on the monocyte population. In experiments assessing the effect of M6P on cellular uptake of sCD26, purified monocytes (0.5×10⁶ monocytes/well) were incubated with the appropriate concentration of M6P in the presence or absence of sCD26-OG for 24 h in standard medium. Cells were then washed twice in ice-cold PBS, followed by incubation for 2 min in acidic PBS (pH 3.0) to strip any sCD26-OG attached to the cell surface. Cells were washed in ice-cold PBS and were incubated with mouse Ig isotypes (1 μg/ml) to block nonspecific binding; this was followed by reaction with PE-conjugated anti-CD14 (10 μg/ml) to gate exclusively on monocyte population.

Flow cytometric analysis of 10,000 viable cells was conducted on FACSCalibur (BD Biosciences, Mountain View, Calif.). Each experiment was repeated at least three times, and the results were provided in the form of a histogram of a representative experiment, or increased mean percent±SE fluorescent intensity compared with control or untreated cells.

Confocal Laser Microscopy

For fluorescent microscopy studies of uptake of sCD26 by monocytes; purified monocytes were incubated with sCD26-OG (1 μg/ml) with or without preincubation with. TT (0.5 μg/ml). Cells were then washed in ice-cold PBS twice and were incubated in acidic PBS (pH 3.0) to strip any sCD26-OG attached to the cell surface. The cells were then attached to microslide glass (Matsunami Glass, Tokyo, Japan) and fixed with 3% paraformaldehyde in PBS for 15 min at room temperature. Cells were blocked with mouse Ig isotypes (1 μg/ml) for 30 min at 4° C., followed by incubation with biotinylated anti-CD14 (10 μg/ml) for 30 min at 4° C., then washed with ice-cold PBS twice and incubated with streptavidin-conjugated Texas red conjugate (1/200; Beckman Coulter) for 30 min at 4° C. In studies of sCD26 uptake via M6P/IGF-IIR on monocytes, purified monocytes were incubated with or without TT for 16 h, and sCD26-OG and M6P/IGF-IIR-red were added to the culture well after being blocked with mouse Ig isotypes (1 μg/ml) for 30 min at 4° C. Cells were then incubated for 30 min at 4° C. For detection of cell surface colocalization, cells were washed in ice-cold PBS twice and were attached to microslide glass, followed by fixation with 3% paraformaldehyde in PBS for 15 min at room temperature. For detection of intracellular incorporation, cells were incubated for 24 and 36 h at 37° C. in a 5% CO₂ humidified atmosphere, and were then washed in ice-cold PBS twice and incubated in acidic PBS (pH 3.0) to strip any sCD26-OG attached to the cell surface. The cells were then attached to microslide glass and fixed with 3% paraformaldehyde in PBS for 15 min at room temperature. Confocal microscopy was performed with an Olympus IX70 confocal microscope with 60 objective lenses (Olympus, Tokyo, Japan), using laser excitation at 496 and 568 nm. The widths of Oregon green and Texas red emission channels were set to maximize specificity.

Relative Quantitative RT-PCR Assay

In studies assessing expression of CD86 mRNA, purified TT-treated monocytes (0.5×10⁶ monocytes/well) were incubated with sCD26 (0.5 μg/ml) at the appropriate time intervals. RNA was then extracted by the use of TRIzol reagent solution according to the manufacturer's instruction (Life Technologies). cDNA was produced by using Thermo-Script II reverse transcriptase (Life Technologies) with oligo(dT)₁₂₋₁₈ primers. The quantities of mRNA were adjusted equally by using PCR of β-actin. Forward primer was 5′-CAAGAGATGGCCACGGCTGCT-3′ (cDNA position 746-766: SEQ ID NO: 38), and reverse primer was 5′-TCCTTCTGCATCCTGTCGGCA-3′ (cDNA-position 1000-1020: SEQ ID NO: 39)) as the internal control. CD86 mRNA was amplified with primers designed to amplify the entire coding sequence of CD86 (forward primer was 5′-ATGGGACTGAGTAACATTCTCTTTGTGATGGCCT-3′ (cDNA position 149-181 and linker: SEQ ID NO: 40), and reverse primer was 5′-CTCGAGTTAAAAACATGTATCACTTIGTCGCATGA-3′ (cDNA position 1090-1120 and linker: SEQ ID NO: 41)). The PCR were performed as follows: 94° C. for 4 min, then denaturing at 94° C. for 30 s, annealing at 64° C. for 1 min, and extending at 72° C. for 30 s at the different cycles, followed by a final extension at 72° C. for 5 min. Amplified DNA was then electrophoresed on a 3% agarose gel and was stained with ethidium bromide.

Blocking Assay by mAbs and Human CTLA-4 Ig

For blocking studies, monocytes were incubated with mAbs against CD80, CD86, HLA-DR, or CTLA-4 Ig at various concentrations for 15 min followed by the addition to T cells. mAbs or Ig were left during the entire culture period. Relevant mAbs or control murine Ig were used as isotype controls. Inhibitory effects on T cell proliferation were expressed as the percentage of reactivity of control cultures without the addition of blocking mAbs or Ig, which were performed in parallel.

Statistics

Student's t test was used to determine whether the difference between control and sample was significant p<0.05 being significant).

Results

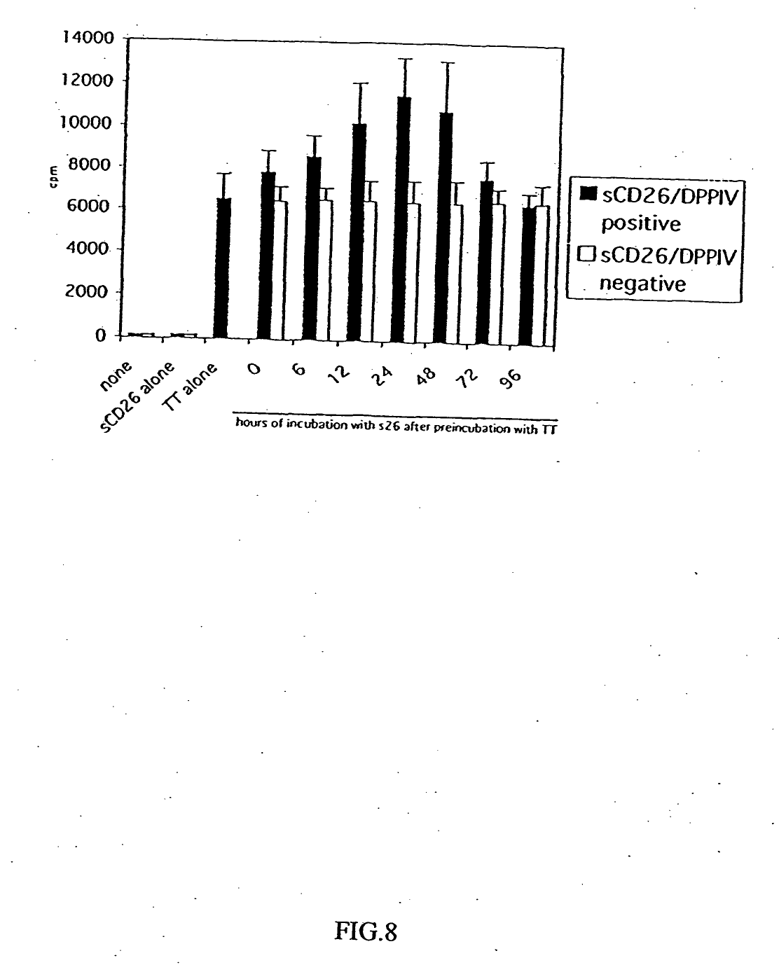

sCD26 enhances T cell Proliferation in the Early Stage of Immune Response to Recall Ag. To investigate the mechanism involved in the enhancement of TT-induced T cell proliferation by sCD26, the inventors first performed a time-course analysis by adding sCD26 to the PBMC system stimulated by TT in vitro. In the early stage of the immune response to foreign antigens, direct interaction between APC and T cells is indispensable (Hathcock et al., 1994; Yi-qun et al., 1996; Hakamada-Taguchi et al., 1998). However, in the latter stages, direct APC-T cell interaction is not always necessary, whereas secreted cytokines such as IL-2 are essential in maintaining the reaction (McAdam et al., 1998; Hathcock et al., 1994). As shown in FIG. 8, the addition of sCD26 within first 48 h resulted in an enhancement in T cell proliferation to TT. In contrast, the addition of sCD26 after 48 h did not enhance TT-mediated activation, and an enhancing effect of TT-induced T cell proliferation was not observed in the experiments with the DPPIV-deficient mutant sCD26. These results indicate that sCD26 affected the early stages of immune reaction to TF but did not have an appreciable effect on the latter stages. Moreover, these data are consistent with previous studies showing that the enhancement of TT-induced T cell proliferation requires DPPIV enzyme activity (Tanaka et al., 1993).

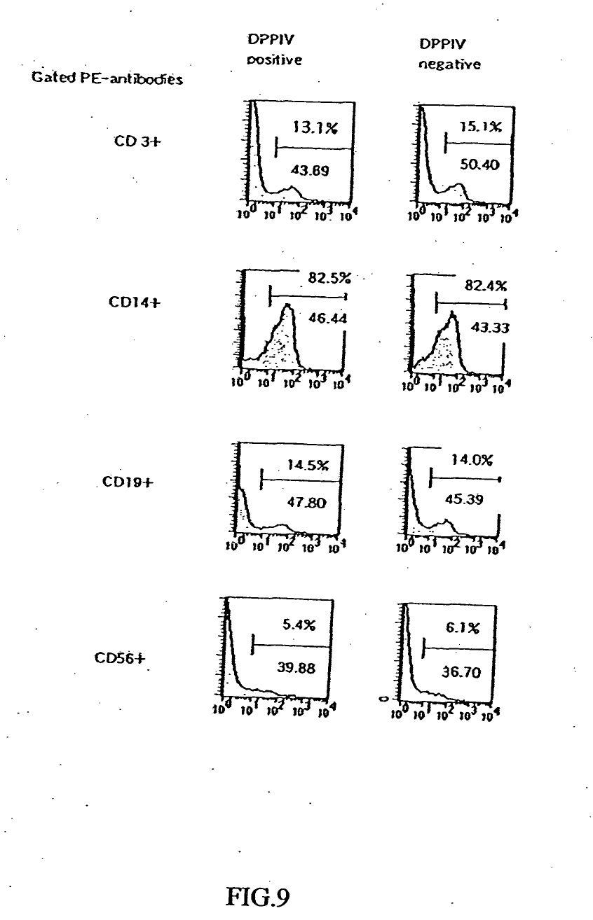

Incubation with sCD26 Leads to Uptake by Monocytes. Because sCD26 affected the early stages of immune response to TT, the inventors next attempted to determine the target cells of sCD26. For this purpose, the inventors incubated PBMC with sCD26-OG for 24 h in the presence or absence of TT. As shown in Table 8, leukocyte phenotypes such as CD3, CD14, CD19, and CD56 were not affected by presence of TT. In addition, sCD26 was taken up mainly by CD14-positive monocytes (Table 8 and FIG. 9). In contrast, flow cytometric analyses showed that T cells (CD3⁺), B cells (CD19⁺), and NK cells (CD56⁺) displayed relatively low levels of sCD26. It should be noted that the above findings were observed both in the presence and absence of TT, and that DPPIV-deficient mutant sCD26 (sCD26/DPPIV⁻) was also preferentially taken up by CD14-positive monocytes. Additional evidence that sCD26 was taken up by monocytes was seen in studies involving confocal microscopy. Concordant data shown in Table 8 and. FIG. 9, show that sCD26/DPPIV⁺, as well as sCD26/DPPIV⁻, was clearly taken up by monocytes. However, sCD26ADPPIV⁺, sCD26/DPPIV⁻, sCD26 was no longer detectable intracellularly 36 h after incubation of monocytes with sCD26. The disappearance of sCD26 molecules following uptake by monocytes was also observed by flow cytometric analysis. It should be noted that this uptake of sCD26 by monocytes was not affected by the presence of TT.

TABLE 8
SCD-26-OG Incorportion According to Lymphocyte Subpopulations^{a}

| | | | | | |
|---|---|---|---|---|---|
| Gated | PE-ab | CD3⁺ | CD14⁺ | CD⁺ | Cd56⁺ |

% Cells with DPPIV⁺ sD26-OG

| | | | | | |
|---|---|---|---|---|---|
| TT⁻ | % | 14.4 ± 0.5 | 80.3 ± 5.1 | 15.4 ± 1.8 | 5.6 ± 1.0 |
| | MF1 | 47.9 ± 4.8 | 51.5 ± 2.7 | 49.2 ± 4.1 | 48.8 ± 4.8 |
| TT⁺ | % | 13.8 ± 1.6 | 85.8 ± 5.6 | 16.1 ± 1.9 | 4.9 ± 1.1 |
| | MF1 | 44.5 ± 2.9 | 48.5 ± 3.6 | 47.4 ± 4.8 | 40.3 ± 5.2 |

% Cells with DPPIV⁻ sD26-OG

| TT⁻ | % | 15.8 ± 1.5 | 83.1 ± 4.9 | 14.9 ± 2.8 | 6.6 ± 1.3 |
|---|---|---|---|---|---|
| | MF1 | 50.4 ± 4.1 | 50.6 ± 4.3 | 49.8 ± 3.2 | 42.7 ± 5.1 |
| TT⁺ | % | 13.2 ± 1.4 | 82.4 ± 4.3 | 17.3 ± 2.9 | 3.7 ± 1.4 |
| | MF1 | 49.4 ± 3.5 | 44.1 ± 6.2 | 47.8 ± 3.7 | 46.8 ± 6.6 |
| ^{a}Freshly isolated PBMC (1 × 10⁶/well) were incubated with or without TT for 16 h after 24-h incubation, and then sCD26/DPPIV⁺ -OG was added to the culture wells. After incubation with sCD26-OG for 24 h, cells were washed in ice-cold PBS, and in acidic buffer (pH3 PBS) for stripping the sCD26-OG on the cell surfaces. Then, cells were incubated with various PE-conjugated Abs (CD3, CD14, CD19, and CD56) for 30 min. Analysis was performed by FACSCalibur. | | | | | |
| # The studies represent mean values ± SE calculated from three independently performed studies. %, Percent of sCD26-OG positive cell number in the various PE-positive cells. Similar studies were performed using mutant sCD26 of defective DPPIV activity (sCD26/DPPIV⁻OG). MFI, mean fluorescence intensity. | | | | | |

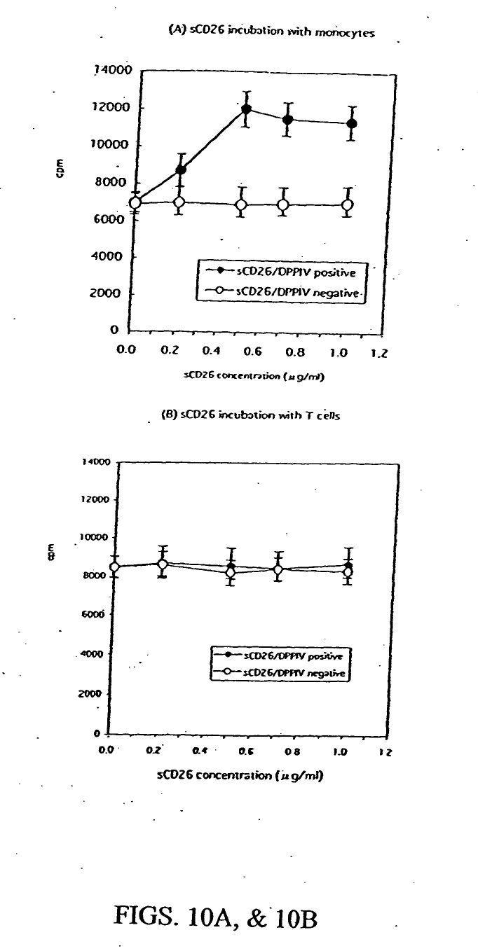

The target cells of sCD26 are monocytes. Because the main target cells among PBMC were monocytes, as shown in Table 8 and FIG. 9, the inventors next attempted to confirm the enhancement of TT-induced T cell proliferation by monocytes that take up sCD26. For this purpose, a reconstitution study was performed by separating T cells and monocytes at the time of incubation with sCD26. As shown in FIGS. 10A & 10B, the enhancing effect of TT-induced T cell proliferation was observed only when monocytes were preincubated with TT and sCD26, but not T cells (FIG. 10A and FIG. 10B). Importantly, these studies again confirmed that sCD26-mediated enhancement of TT-induced T cell proliferation required DPPIV enzyme activity. To further confirm that sCD26 uptake by TT-primed monocytes leads to enhancement of T cell proliferation, the inventors performed a reconstitution study at different doses of sCD26. As shown in FIG. 10A, the degree of TT-induced T cell proliferation was dependent upon the concentration of the exogenously added sCD26. Therefore, these results indicate that the principal target cells of sCD26 are APCs, including monocytes.

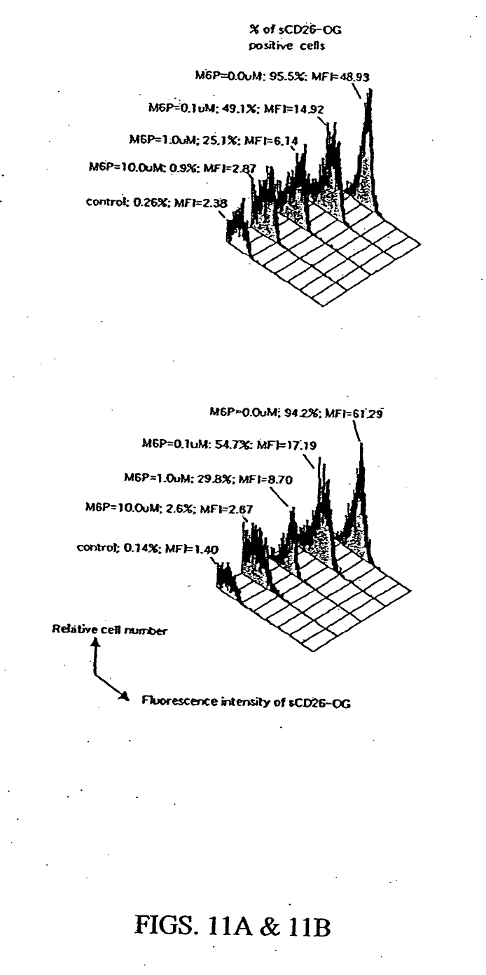

Uptake of sCD26 into Monocytes Occurs via its Binding to M6P/IGF-IIR. The inventors recently showed that M6P/IGF-IIR was the binding protein for CD26 and that it played a role in internalizing CD26 molecule into T cells after ligation of CD26 (Ikushima et al., 2000). To examine whether M6P/IGF-IIR is involved in monocyte uptake of sCD26, fluorescent confocal microscopy was used to initially evaluate monocyte expression of sCD26-OG and M6P/IGF-IIR intracellularly and on the cell surface. For this purpose, fluorescent mouse anti-human M6P/IGF-IIR mAb was conjugated with M6P/IGF-IIR-red. It was shown that sCD26-OG and M6P/IGF-IIR-red colocalized on the monocyte cell surface. Following incubation at 37° C. for 24 h in the presence of sCD26-OG and M6P/IGF-IIR-red, intracellular colocalization of these proteins was observed. Colocalization of lysozyme and M6P/IGF-IIR was similarly observed. In contrast, colocalization was not observed after incubation of sCD26-OG and Texas red-conjugated mouse IgG1 to exclude the possibility of nonspecific binding to FcγR. To further confirm that sCD26 uptake is dependent on its binding to M6P/IGF-IIR, the inventors have performed the inhibition assay to evaluate uptake of sCD26 by monocytes in the presence of excess amounts of M6P. As shown in FIG. 11A and FIG. 11B, FACS analyses showed that the addition of M6P into the culture system (0, 0.1, 1.0, and 10 μM) inhibited the uptake of sCD26 into monocytes. The degree of inhibition of sCD26 uptake was dependent upon the concentration of the exogenously added M6P (FIG. 11A). These findings were also observed even in the absence of TT (FIG. 11B). Thus, these results indicated that uptake of sCD26 into monocytes as due to the interaction of sCD26 with its binding protein M6P/IGF-IIR. It should be noted that this inhibitory effect of M6P on uptake of sCD26 was similarly seen in experiments performed with sCD26/DPPIV.

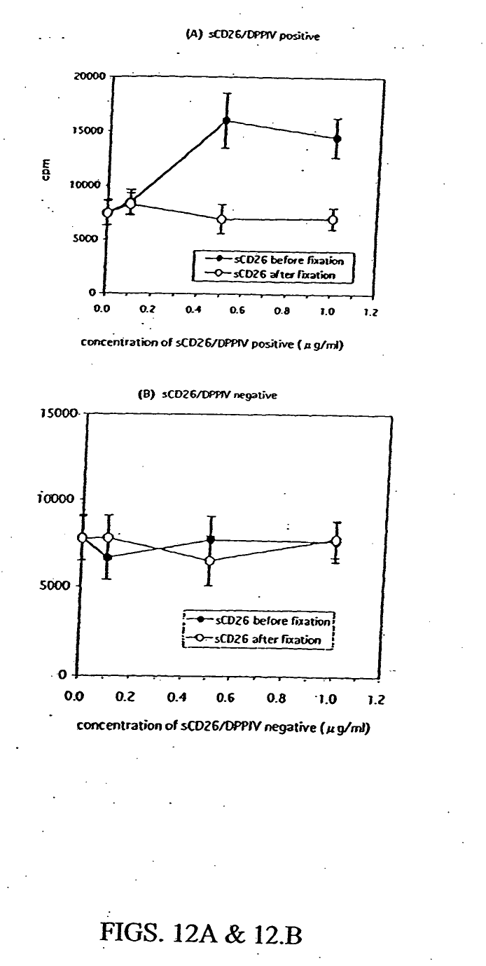

Enhancement of TT-induced T cell proliferation by sCD26 is seen when monocytes are incubated with sCD26 before fixation. Another explanation for the sCD26-induced enhancement of TT-mediated T cell proliferation following monocyte uptake may be the trimming of the MHC class II-bound peptide, hence altering cellular responsiveness to the Ag. Such a phenomenon has been described previously with CD 13 aminopeptidase N (Tanaka et al., 1994). CD13 contributes to Ag processing by trimming the MHC class II-bound peptide on the APC surface. Because CD26, like CD13, is also an ectopeptidase, it may have the effect of trimming MHC class II-bound peptide on the surface of APC. To evaluate this issue, the inventors fixed TT-pulsed monocytes before and after incubation with sCD26, and subsequently examined whether the enhancing effect of sCD26 on T cell activation was observed. As shown in FIG. 12A and FIG. 12B, an enhanced T cell proliferation was seen only when the monocytes were incubated with sCD26/DPPIV⁺ before fixation. In contrast, monocytes incubated with sCD26 after fixation did not alter the immune response occurring after the Ag peptide bound to MHC molecule. Therefore, these results suggested that enhancement of TT-induced T cell proliferation by sCD26 does not result from trimming of the MHC class II-bound peptide on the surface of APC. Rather, it is likely that internalization of sCD26 into monocytes affects the interaction of monocytes and T cells.

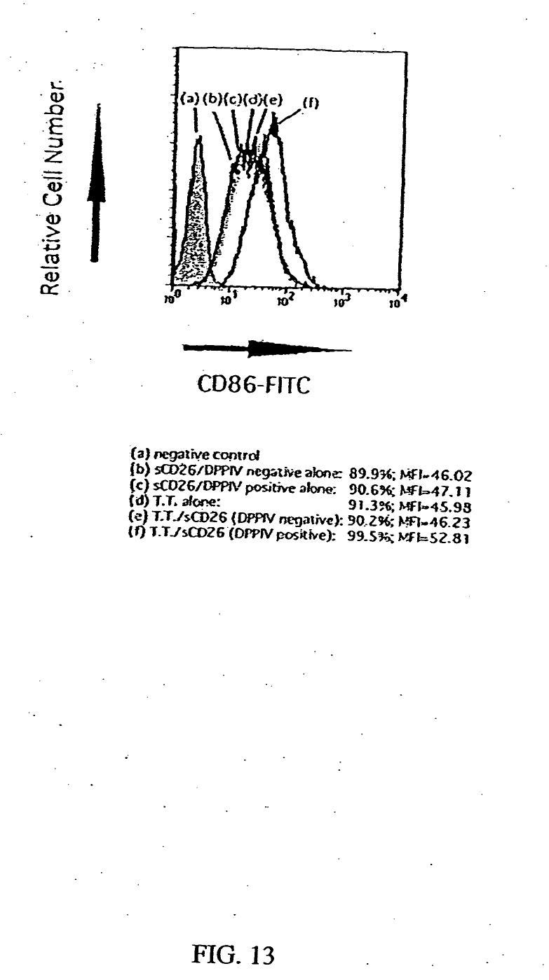

Expression of the Costimulatory Molecule CD86 is Increased on Monocytes Following Exposure to sCD26. The inventors next analyzed the expression of several surface molecules on monocytes that have been previously described to play a role in T cell/monocyte interaction (Lenschow et al., 1996; Yokochi et al., 1982; Azuma et al., 1993; Freeman et al., 1993; McAdam et al., 1998; Chambers, 2001; Hathcock et al., 1994; Yi-Qun et al., 1996; Hakamada-Taguchi et al., 1998; Manickasingham et al., 1998). For this purpose, freshly isolated monocytes were incubated with sCD26 in the presence or absence of TT, and the expression of CD80, CD86, and HLA-DR on monocytes was examined using flow cytometric analysis. As shown in Table 9 and FIG. 13, CD86 molecule expression on monocytes was increased within the first 48 h after TT and sCD26 stimulation. However, the increase in CD86 expression was no longer observed after 48 h of sCD26 incubation. In contrast, expression of CD80 and HLA-DR was not affected by stimulation of TT and sCD26 (Table 8). Therefore, these results showed that uptake of sCD26 into monocytes resulted in an increase in CD86 expression when monocytes were pulsed with TT and sCD26. Furthermore, these findings suggested that the enhanced CD86 expression on sCD26-treated monocytes contributed to trigger TT-induced T cell proliferation in the early stages of the immune response to recall Ag. It should be noted that sCD26/DPPIV⁻ mutant did not affect CD86 expression on monocytes, consistent with data showing that only sCD26/DPPIV⁺, but not sCD26/DPPIV⁻ mutant, enhanced TT-induced T cell proliferation Tanaka et al., 1993; Manickasingham et al., 1998).

Enhanced Expression of CD86 on Monocytes Triggered by TT and sCD26 is Dependent on Increased Synthesis of mRNA. To determine whether the enhancement in CD86 expression following incubation of monocytes with sCD26 in the presence of TT is dependent on increased protein synthesis, levels of mRNA encoding for CD86 were quantified by RT-PCR. Freshly isolated monocytes were incubated with or without TT for 16 h after a 24-h incubation in the standard medium alone, and then sCD26 (0.5 μg/ml) was added to the culture wells. After incubation with sCD26 for 24 h, cells were processed for RNA isolation as described in Materials and Methods. The increase of surface CD86 expression seen when monocytes were incubated with TT and sCD26/DPPIV⁺ is also associated with increased mRNA levels, suggesting that enhanced protein synthesis is one potential mechanism for enhanced CD86 surface expression. Of importance is the fact that monocytes incubated with TT and sCD26/DPPIV⁻ did not demonstrate enhanced CD86 mRNA levels, again indicating the importance of DPPIV activity in this interaction.

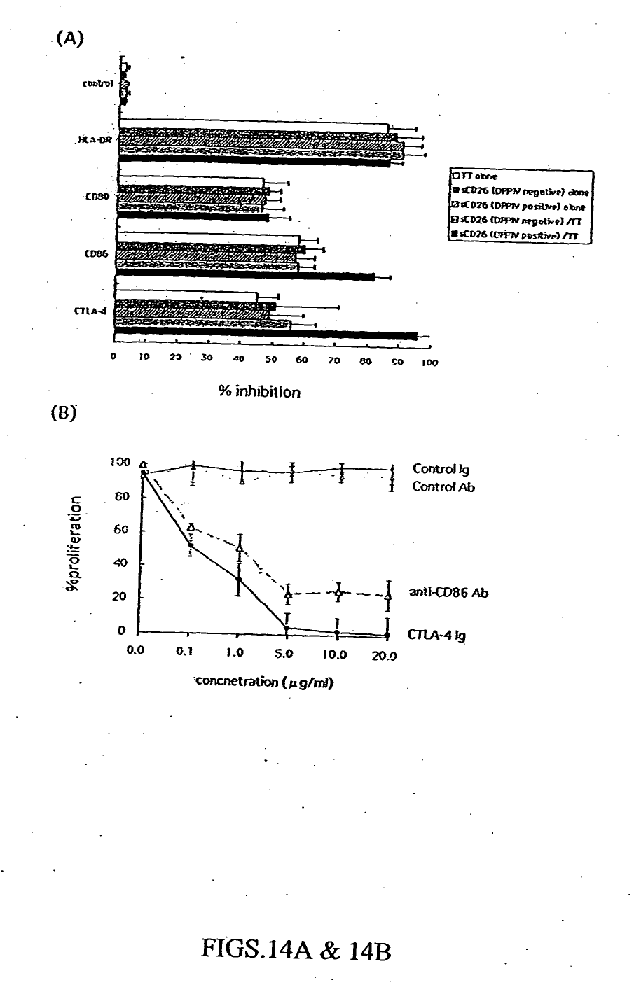

Inhibitory Effects of mAbs and CTLA-4 Ig on T-Cell Proliferation by Monocytes Triggered by TT and sCD26. To further define the role of various surface molecules on T cell proliferation induced by TT-treated monocytes, monocytes were first treated with TT and/or sCD26, and then they were incubated with mAbs against CD80, CD86, HLA-DR, or CTLA4 Ig at a final concentration of 5 μg/ml for 15 min at 4° C. before onset of culture. Cells were then incubated at 37° C. for the entire culture period. As shown in FIG. 14A, although mAb against HLA-DR efficiently inhibited T cell proliferation, sCD26 did not have an effect in the HLA-DR-mediated pathway. Similarly, no effect was observed in the TT-induced T cell proliferation after treatment of monocytes with anti-CD80 in the presence or absence of sCD26. In contrast, as shown in the lower panel of FIG. 14A, whereas T cell proliferation was inhibited by the presence of mAb against CD86 molecule and CTLA-4 Ig, this inhibition was significantly stronger in the culture with TT/sCD26 (sCD26/DPPIV⁺)-treated monocytes. The blocking effect of anti-CD86 Ab and CTLA-4 Ig was not exerted through the inhibition of DPPIV activity of sCD26, as examined in liquid phase by ELISA. To confirm whether the above costimulatory effect was observed via the induction of CD86, CD86 mAb and CTLA-4 Ig were added at different concentrations to TT and/or sCD26 stimulation cultures. As shown in FIG. 14B TT-mediated T cell proliferation enhanced by sCD26/DPPIV⁺ was strongly inhibited by CD86 mAb and CTLA-4 Ig in a dose-dependent manner. It should be noted that CTLA-4 Ig always exerted greater inhibitory effect on TT-mediated T cell proliferation than CD86 mAb. As previously noted in FIG. 13, TT/sCD26 (DPPIV⁺) monocytes expressed higher levels of CD86 than monocytes incubated with sCD26/DPPIV⁻ molecules in the presence or absence of TT. These data strongly suggested that the increased surface expression of CD86 on monocytes treated with TT and sCD26 (DPPIV⁺) is essential for the enhancing effect of sCD26 on TT-induced T cell proliferation.

Role of CD26 in Immune System Potentiation

This Examples demonstrates that CD26 has an enhancing effect on T cell proliferation via the activation of antigen presenting cells.

An increase in the surface expression of the costimulatory molecule CD86 on monocytes was shown following uptake of sCD26. This immune enhancing effect of CD26 was observed within the initial 48 h of treatment with CD26 in the presence of the TT antigen. In the process of T cell proliferative response against a recall Ag, several major factors have been shown to contribute to the maintenance of the biological reaction, including APC, Th cells, and selected cytokines (Lenschow et al., 1996; Yokochi et al., 1982; Azuma et al., 1993; Freeman et al., 1993; McAdam et al., 1998; Chambers, 2001; Hathcock et al., 1994; Yi-Qun et al., 1996; Hakamada-Taguchi et al., 1998; Manickasingham et al., 1998). Initially, an APC-T cell interaction plays a key role in triggering the T cell response, leading eventually to expression of the T cell biological program (Lenschow et al., 1996; McAdam et al., 1998; Chambers, 2001). The present invention shows that the enhancing effect of sCD26 on TT-induced T cell proliferation occurrs in the early stages of the immune response. Moreover, the cells affected by exogenously added sCD26 are the CD14-positive monocytes.

CD28 is constitutively expressed on T cells and interacts with the B7 molecules CD80 and CD86 (Caux et. al., 1994; Hathcock et al., 1994). This interaction leads to increased T cell proliferation, IL-2 production, and resistance to apoptosis (McAdam et al., 1998). CD80 and CD86 are type 1 membrane glycoproteins belonging to the Ig superfamily (Azuma et al., 1993; Freeman et al., 1993). In humans, their expression patterns differ according to the nature of the APC. CD86 expression is constitutive on monocytes and dendritic cells and is up-regulated by activation (Lenschow et al., 1996; McAdam et al., 1998). In contrast, CD80 is expressed at low levels on APC and is up-regulated following activation (McAdam et al., 1998; Chambers, 2001).

CD86 has an important role in the priming of naive T cells and activation of memory T cells (Lenschow et al., 1996; McAdam et al., 1998; Saito, 1998; Engel et al., 1994; Vyth-Dreese et al., 1995; Yokozeki et al., 1996). Activation of naive T cells requires strong stimulatory signals provided by APC (Liu and Janeway, 1992), and activation of recently activated memory T cells can be elicited with anti-CD3 mAb alone (Van de Velde et al., 1993), most memory T cells are still dependent on CD28 triggering for their activation (Yi-qun et al., 1996). A stable interaction between APC and T cells is dependent not only on the absolute affinity and specificity on the TCR and its ligands, but also on the relative density of molecules available for contact at the interaction site (Prakken et al., 2000; Grakoui et al., 1999). Therefore, the findings of the present invention that up-regulation of CD86 but not CD80 on monocytes occurs following uptake of sCD26 or expression of CD26 in a immune cell population demonstrates that CD26 activates antigen presenting cells.

It is possible that CD26/DPPIV exerts its effect via the membrane-bound form, particularly in T cells. It is also possible that the CD26/DPPIV exerts its effect with the soluble form, in view of the fact that CD26/DPPIV is actually present in human serum (Tanaka et al., 1994). Therefore both sCD26 as well as non-soluble forms are important.

Cytokines are critical in maintaining the latter stages of immune reaction (Lenschow et al., 1996; McAdam et al., 1998; Yi-qun et al.,: 1996). In the initial stages of Ag presentation, the capacity of Ag presentation can be modulated by several nonexclusive mechanisms, including the efficiency of Ag capturing and loading, MHC molecule density and occupancy, or altered costimulatory molecule expression (Prakken et al., 2000; Grakoui et al., 1999). The present example demonstrates that sCD26 is taken up by monocytes and exertes its enhancing effect on T cell proliferation by altering the Ag presenting function of monocytes through the up-regulation of CD86 expression. As antigen presenting cells activate both Thelper cells as well as CTL's and B cells, CD26 is an important effector in potentiating immune responses via the APC and has immense therapeutic utility.

TABLE 9
Increased CD86 Expression on Monocytes After Incubation with sCD26/DPPVI^{+a}

| | | | | | | | |
|---|---|---|---|---|---|---|---|
| TT alone | SCD26 alone | 0 h | 6 h | 24 h | 48 h | 72 h | 96 h |

% Increase of CD86 expression on monocytes following incubation with sCD26

| | | | | | | | | |
|---|---|---|---|---|---|---|---|---|
| SCD26/DPPV⁺ | 1.2 ± 2 | 1.5 ± 0.6 | 4.2 ± 0.8 | 7.4 ± 1.3 | 10.5 ± 2.4 | 12.1 ± 2.1 | 4.3 ± 1.2 | 4.1 ± 1.5 |
| SCD26/DPPV⁻ | | 1.8 ± 1.3 | 2.4 ± 1.9 | 1.2 ± 1.1 | 0.8 ± 1.4 | 1.6 ± 1.1 | 1.3 ± 0.8 | 1.1 ± 0.7 |

% Increase of CD80 expression on monocytes following incubation with sCD26

| | | | | | | | | |
|---|---|---|---|---|---|---|---|---|
| SCD26/DPPV⁺ | 2.0 ± 0.4 | 1.7 ± 0.2 | 1.3 ± 0.1 | 0.7 ± 0.3 | 1.6 ± 0.4 | 0.6 ± 0.2 | 0.8 ± 0.2 | 0.5 ± 0.3 |
| SCD26/DPPV⁻ | | 1.0 ± 0.4 | 0.3 ± 0.2 | 0.2 ± 0.1 | 0.2 ± 0.1 | 0.1 ± 0.1 | 0.2 ± 0.1 | 0.2 ± 0.1 |

% Increase of HLA-DR expression on monocytes following incubation with sCD26

| SCD26/DPPV⁺ | 0.3 ± 0.2 | 1.5 ± 0.6 | 0.2 ± 0.8 | 1.4 ± 1.5 | 3.5 ± 2.6 | 2.1 ± 1.1 | 0.4 ± 1.2 | 1.1 ± 1.5 |
|---|---|---|---|---|---|---|---|---|
| SCD26/DPPV⁻ | | 2.8 ± 1.3 | 1.4 ± 0.7 | 0.2 ± 1.1 | 1.4 ± 0.7 | 0.8 ± 1.2 | 0.7 ± 0.8 | 2.3 ± 1.7 |
| ^{a}The freshly isolated monocytes (0.5 × 10⁶/well) were incubated with or without TT for 16 h after 24-h incubation, and then sCD26(0.5 μg/ml) was added to the culture wells. After incubation for different time intervals (0.6, 24, 48, 72, and 96 h) cells were washed in ice-cold PBS, and incubated with FITC-conjugated CD86, CD80, or HLA-DR on ice for 30 min. After washing with PBS, cells were | | | | | | | | |
| # analyzed by FACSCalibur. The studies represent mean values ± SE calculated from three independently performed studies. %, Percent increase of FITC intensity compared to FITC intensity of mon-sCD26-treated monocytes. | | | | | | | | |

### Example 6

### Clinical Trials

This section is concerned with the development of human treatment protocols for anticancer therapy using the CD26 compositions in combination with chemotherapeutic/radiotherapeutic agents and optionally other anticancer therapeutic agents. Although only cancer related treatments are described here, this Example, is also applicable to the treatment of immune diseases such as potentiating immune responses during infections, immunosupressive conditions, cancers etc. as CD26 enhances presentation of antigens by APC's to T-cells and thereby causes proliferation of activated T-cells.

The various elements of conducting a clinical trial, including patient treatment and monitoring, will be known to those of skill in the art in light of the present disclosure. The following information is being presented as a general guideline for use in establishing the CD26-based therapy described herein alone or in combinations with other adjunct treatments used routinely in cancer therapy in clinical trials.

Candidates for the phase 1 clinical trial will be patients on which all conventional therapies have failed. Approximately 100 patients will be treated initially. Their age will range from 16 to 90 (median 65) years. Patients will be treated, and samples obtained, without bias to sex, race, or ethnic group. For this patient population of approximately 41% will be women, 6% will be black, 13% Hispanic, and 3% other minorities. These estimates are based on consecutive cases seen at MD Anderson Cancer Center over the last 5 years.

Optimally the patient will exhibit adequate bone marrow function (defined as peripheral absolute granulocyte count of >1,000/mm³ and platelet count of 100,000/mm³ (unless decreased due to tumor involvement in the marrow), adequate liver function (bilirubin ≦1.5 mg/dl, SGOT/SGPT <4× Upper Limit of Normal) and adequate renal function (creatinine ≦1.5 mg/dl).

Research samples will be obtained from peripheral blood or marrow under existing approved projects and protocols. Some of the research material will be obtained from specimens taken as part of patient care.

The CD26 compositions and formulations in combination with chemotherapeutic/radiotherapeutic agent(s) treatments described above will be administered to the patients regionally or systemically on a tentative weekly basis. A typical treatment course may comprise about six doses delivered over a 7 to 21 day period. Upon election by the clinician the regimen may be continued with six doses every three weeks or on a less frequent (monthly, bimonthly, quarterly, etc.,) basis. Of course, these are only exemplary times for treatment, and the skilled practitioner will readily recognize that many other time-courses are possible.

The modes of administration may be local administration, including, by intratumoral injection and/or by injection into tumor vasculature, intratracheal, intrathecal, endoscopic, subcutaneous, and/or percutaneous. The mode of administration may be systemic, including, intravenous; intra-arterial, intra-peritoneal and/or oral administration.

The CD26 compositions may be administered such that final dosages in the range of of 0.001 microgram/ml to 1 gram/ml are delivered, although exact effective dosage will depend on subsequent testings. In some embodiments the CD26 compositions are administered as liposomal formulations or potentially via other artificial carriers. For example, a liposomal formulation of the CD26 is administered a range of 0.001 μg/ml to 1 mg/ml intravenously or by other routes described. The CD26 compositions may also be administered in conjunction with targetting agents that will target the CD26 composition to tumor cells. Such methods of targetting are well known in the art and described supra.

Of course, the skilled artisan will understand that while these dosage ranges, provide useful guidelines appropriate adjustments in the dosage depending on the needs of an individual patient factoring in disease, gender, performance status, age and other general health conditions will be made at the time of administration to a patient by a trained physician. The same is true for means of administration, routes of administration as well.

To monitor disease course and evaluate the cancer cell killing it is contemplated that the patients should be examined for appropriate tests every month. To assess the effectiveness of the drug, the physician will determine parameters to be monitored depending on the type of cancer/tumor and will involve methods to monitor reduction in tumor mass by for example computer tomography (Cl) scans, PET scans, gallium scans, detection of the presence of the tumor antigens on cell surface and in serum such as PSA (prostrate specific antigen) in prostrate cancer, HCG in germ tumor, CEA in colon cancer, CA125 in ovarian cancer, LDH and B2 microglobulin in lymphomas, and the like. Tests that will be used to monitor the progress of the patients and the effectiveness of the treatments include: physical exam, X-ray, blood work, bone marrow work and other clinical laboratory methodologies. The doses given in the phase 1 study will be escalated as is done in standard phase 1 clinical phase trials, i.e. doses will be escalated until maximal tolerable ranges are reached.

Clinical responses may be defined by acceptable measure. For example, a complete response may be defined by complete disappearance of the cancer cells whereas a partial response may be defined by a 50% reduction of cancer cells or tumor mass.

The typical course of treatment will vary depending upon the individual patient and disease being treated in ways known to those of skill in the art. For example, a patient with colon-cancer might be treated in four week cycles. The duration of treatment will similarly be varied, although potentially longer duration may be used if no adverse effects are observed with the patient, and shorter terms of treatment may result if the patient does not respond or suffers from intolerable toxicity.

All of the compositions and/or methods disclosed and claimed herein can be made and executed without undue experimentation in light of the present disclosure while the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following-references, to the extent that they provide exemplary procedural or other details supplementary to those set forth herein, are specifically incorporated herein by reference.
U.S. Pat. No. 4,554,101
U.S. Pat. No. 4,682,195
U.S. Pat. No. 4,683,202
U.S. Pat. No. 5,252,479
U.S. Pat. No. 5,639,793
U.S. Pat. No. 5,747,520
U.S. Pat. No. 6,156,791
U.S. Pat. No. 6,207,673
U.S. Pat. No. 6,300,090
Angel et al. , Cell, 49:729, 1987b.
Angel et al. , Mol. Cell. Biol., 7:2256, 1987a.
Ariumi et al., FEBS Lett., 436:288-292, 1998.
Atchison and Perry, Cell, 46:253, 1986.
Atchison and Perry, Cell, 48:121, 1987.
Aytac et al., Cancer Research, 61:7204-7210, 2001.
Azuma et al., Nature 366:76, 1993.
Baichwal and Sugden, In: Gene Transe, Kucherlapati (ed.), NY, Plenum Press, 117-148, 1986.
Banerji et al., Cell, 27(2 Pt 1):299-308, 1981.
Baneiji et al., Cell, 33(3):729-740, 1983.
Barany and Merrifield, In: The Peptides, Gross and Meienhofer (eds.), Academic Press, NY, 1-284, 1979.
Basanez et al., J. Biol. Chem., 276:31083-31091, 2001.
Bauvois et al., Br. J. Cancer, 79:1042-1048, 1999.
Beck et al., Drug Resist. Updat., 2:392-389, 1999.
Benvenisty and Neshif, Proc. Natl. Acad. Sci. USA, 83(24):9551-9555, 1986.
Berkhout et al., Cell, 59:273-282, 1989.
Blanar et al., EMBO J., 8:1139, 1989.
Blanchard et al., J. Biol. Chem. 273:20886, 1998.
Blanchard et al., J. Biol. Chem. 274:24685, 1999.
Bodine and Ley, EMBO J., 6:2997, 1987.
Boshart et al., Cell, 41:521, 1985.
Bosze et al., EMBO J., 5(7):1615-1623, 1986.
Braddock et al., Cell, 58:269, 1989.
Bulla and Siddiqui, J. Virol., 62:1437, 1986.
Campbell and Villarreal, Mol. Cell. Biol., 8:1993, 1988.
Campere and Tilghman, Genes and Dev., 3:537, 1989.
Campo et al., Nature, 303:77, 1983.
Capaldi et al., Biochem. Biophys. Res. Comm., 74(2):425-433, 1977.
Carbone et al., Blood, 86:4617-4626, 1995.
Caux et al., J. Exp. Med. 180:1841, 1994.
Celander and Haseltine, J. Virology, 61:269, 1987.
Celander et al., J. Virology, 62:1314, 1988.
Chambers, Trends Immunol. 22:217, 2001.
Chandler et al., Cell, 33:489, 1983.
Chang et al., Hepatology, 14:134A, 1991.
Chang et al., Mol Cell. Biol., 9:2153, 1989.
Chatterjee et al., Proc Natl. Acad Sci. U.S.A., 86:9114, 1989.
Choi et al., Cell, 53:519, 1988.
Cohen et al., J. Cell. Physiol., 5:75, 1987.
Colberre-Garapin et al., J. Mol. Biol., 150:1, 1981.
Costa et al., Mol. Cell. Biol., 8:81, 1988.
Couch et al., Am. Rev. Resp. Dis., 88:394-403, 1963.
Coupar et al., Gene, 68:1-10, 1988.
Cripe et al., EMBO J., 6:3745, 1987.
Croft et al., J. Exp. Med 176:1431, 1992.
Culotta and Hamer, Mol. Cell. Biol., 9:1376, 1989.
Dalal et al., Molecular and Cullular Biol., 19:4465-4479, 1999.
Dandolo et al., J. Virology, 47:55-64, 1983.
Dang et al., Cell. Immunol., 125:42-57, 1990.
Dang et al., J. Exp. Med., 172: 649-652, 1990.
Dang et al., J. Immunol., 144:4092-4100, 1990.
Dang et al., J. Immunol., 145:3963-3971, 1990.
Dang et al., J. Immunol., 147:2825-2832, 1991.
Davies et al., J. Biol. Chemis., 263:17724-17729, 1988.
De Villiers et al., Nature, 312(5991):242-246, 1984.
Dennis et al., Proc. Natl. Acad. Sci. USA, 88:580, 1991.
Deschamps et al., Science, 230:1174-1177, 1985.
Dong et al. J. Immunol., 159: 6070-6076, 1997.
Dong et al., J. Immunol., 156:1349-1355, 1996.
Drake et al., Biochem., 28:8154, 1989.
Dubensky et al., Proc. Nat. Acad, Sci. USA, 81:7529-7533, 1984.
Dunphy, Trends Cell. Biol., 4:202-207, 1994.
Edbrooke et al., Mol. Cell. Biol., 9:1908, 1989.
Edlund et al., Science, 230:912-916, 1985.
Engel et al., Blood, 84:1402, 1994.
EPO 0 273 085
Fechheimer et al., Proc. Natl. Acad. Sci. USA, 84:8463-8467, 1987.
Feng and Holland, Nature, 334:6178, 1988.
Ferkol et al., FASEB J, 7:1081-1091, 1993.
Firak and Subramanian, Mol. Cell. Biol., 6:3667, 1986.
Fleischer, Immunol. Today 15:1800, 1994.
Flentke et al., Proc. Natl. Acad. Sci. USA, 88:1556-1559, 1991.
Foecking and Hofstetter, Gene, 45(1):101-105, 1986.
Freemnan et al., Science 262:909, 1993.
Froelich-Ammon and Osheroff, J. Biol. Chem., 270:21429-21432, 1995.
Fry et al., Cancer Res., 51:6592-6595, 1991.
Fujita et al., Cell, 49:357, 1987.
Fujita et al., Oncogene, 17:1295-1304, 1998.
Fulda et al., Oncogene, 20:1063-1075, 2001.
Gao and Dou, Cell Biochem., 80:53-72, 2000.
Gao and Duo, Mol. Pharmacol, 58:1001-1010, 2000.
Ghosh-Choudhury et al., EMBO J., 6:1733-1739, 1987.
Gibson et al., Mol. Cell Biol., 20:205-212, 2000.
Gilles et al., Cell, 33:717, 1983.
Gloss et al., EMBO J., 6:3735, 1987.
Godbout et al., Mol. Cell. Biol., 8:1169, 1988.
Gomez-Foix et al., J. Biol. Chem., 267:25129-25134, 1992.
Goodboum and Maniatis, Proc. Natl. Acad. Sci. USA, 85:1447, 1988.
Goodbourn et al., Cell, 45:601, 1986.
Grad et al., Drug Resist. Updat., 4:85-91, 2001.
Graham and Prevec, In: Methods in Molecular Biology: Gene Transfer and Expression Protocol, Murray (Ed.), Humana Press, Clifton, N.J., 7:109-128, 1991.
Graham et al, J. General Virology, 36:59-74, 1977.
Grakoui et al., Science, 285:221, 1999.
Greene et al., Immunology Today, 10:272,-1989
Greene et al., J. Biol. Chem., 271:26762, 1996.
Grosschedl and Baltimore, Cell, 41:885, 1985.
Grunhaus et al., Seminar in Virology, 200(2):535-546, 1992.
Hakamada-Taguchi et al., Eur. J. Immunol., 28:873, 1998.
Hansen et al., J. Immunol. Methods, 119:203-210, 1989.
Haridas et al., Proc. Natl. Acad. Sci. USA, 98:5821-5826, 2001.
Haslinger and Karin, Proc. Natl. Acad. Sci. USA, 82:8572, 1985.
Hathcock et al., J. Exp. Med., 180:631, 1994.
Hauber and Cullen, J. Virology, 62:673, 1988.
Hegen et al., Immunology, 90:257-264, 1997.
Hen et al., Nature, 321:249, 1986.
Hensel et al., Lymphokine Res., 8:347, 1989.
Hermonat and Muzycska, Proc. Natl. Acad. Sci. USA, 81:6466-6470, 1984.
Herr and Clarke, Cell, 45:461, 1986.
Hersdorffer et al., DNA Cell Biol., 9:713-723, 1990.
Herz and Gerard, Proc. Natl. Acad. Sci. USA, 90:2812-2816, 1993.
Hess et al., J. Adv. Enzyme Reg., 7:149, 1968.
Hirochika et al., J. Virol., 61:2599, 1987.
Hirsch et al., Mol. Cell. Biol., 10:1959, 1990.
Hitzeman et al., J. Biol Chem., 255:2073, 1980.
Ho et al., Clinical Cancer Res., 7:2031-2040, 2001.
Holbrook et al., Virology, 157:211, 1987.
Holden et al., Am. J. Clinical Path. Am. J. Clinical Path., 104:54-59, 1995.
Holland et al., Biochemistry, 17:4900, 1978.
Horlick and Benfield, Mol. Cell. Biol., 9:2396, 1989.
Hsiang et al., Cancer Res. 48:3230-3235, 1988.
Huang et al., Cell, 27:245, 1981
Hug et al., Mol. Cell. Biol., 8:3065, 1988.
Hwang et al., Mo. Cell. Biol., 10:585, 1990.
Ikushima et al., Proc. Natl. Acad. Sci. USA, 97:8439-8444, 2000.
Imagawa et al., Cell, 51:251, 1987.
Imbra and Karin, Nature, 323:555, 1986.
Imler et al., Mol. Cell. Biol, 7:2558, 1987.
Imperiale and Nevins, Mol. Cell. Biol., 4:875, 1984.
Inouye and Inouye, Nucleic Acids. Res., 13:3101-3109, 1985.
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
Jazirehi et al., Clin. Cancer Res., 7:3874-3883, 2001.
Johnson et al., In: Biotechnology And Pharmacy, Pezzuto et al., Eds., Chapman and Hall, NY, 1993.
Johnson et al., Mol. Cell. Biol., 9:3393, 1989.
Jones and Shenk, Cell, 13:181-188, 1978.
Jones et al., Am. J. Clin. Pathol., 115:885-892, 2001.
Jones, Genetics, 85: 12, 1977.
Kadesch and Berg, Mol. Cell. Biol., 6:2593, 1986.
Kahne et al., Cell. Immunol., 189:60, 1998.
Kajiyama et al., Cancer Res., 62:2753-2757, 2002.
Kameoka et al., Science, 261:466-469, 1993.
Karin et al., Mol. Cell. Biol., 7:606, 1987.
Karlsson et. al., EMBO J., 5:2377-2385, 1986.
Katinka et al., Cell, 20:393, 1980.
Kaufmann, Biochim. Biophys. Acta, 1400:195-211, 1998.
Kawamoto et al., Mol. Cell. Biol., 8:267, 1988.
Kellner et al., Lancet. Oncol., 3:235-243, 2002.
Kiledjian et al., Mol. Cell. Biol., 8:145, 1988.
King et al., Cell, 79:563-571, 1994.
Kingsman et al., Gene, 7: 141, 1979.
Klamut et al., Mol. Cell. Biol., 10:193, 1990.
Kluck et al., Science, 275:1132-1136, 1997.
Koch et al., Mol. Cell. Biol., 9:303, 1989.
Koreeda et al., Oral. Biol, 46:759-766, 2001.
Korkolopoulou et al., Histopathology, 38:45-53, 2001.
Kornfeld, Annu. Rev. Biochem. 61:307, 1992.
Korom et al., Transplantation 63:1495, 1997.
Korom et al., Transplantation, 63:1495-1500, 1997.
Kriegler and Botchan, In: Eukaryotic Viral Vectors, Y. Gluzman, ed., Cold Spring Harbor: Cold Sprig Harbor Laboratory, NY, 1982.
Kriegler and Botchan, Mol. Cell. Biol., 3:325, 1983.
Kriegler et al., Cell, 38:483, 1984.
Kriegler et al., Cell, 53:45, 1988.
Krummel, and Allison, J. Exp. Med. 182:459, 1995.
Kubota et al., Clin. Exp. Immunol. 89:19, 1992.
Kuhl et al., Cell, 50:1057, 1987.
Kuida, Int. J. Biochem. Cell. Biol., 32:121-124, 2000.
Kunz et al., Nucl. Acids Res., 17:1121, 1989.
Kyte and Doolittle, J Mol Biol, 157(1):105-32, 1982.
Larsen et al., Proc Natl. Acad. Sci. USA., 83:8283, 1986.
Laspia et al., Cell, 59:283, 1989.
Latimer et al., Mol. Cell. Biol., 10:760, 1990.
Lauber et al., J. Biol. Chem., 276:29772-29781, 2001.
Le Gal La Salle et al., Science, 259:988-990, 1993.
Lee and Nathans. J. Biol. Chem. 263:3521, 1988.
Lee et al., Am. J. Physiol. 268:C127, 1995.
Lee et al., Nature, 294:228, 1981.
Lee et al., Nucleic Acids Res., 12:4191-206, 1984.
Lenschow et al., Annu. Rev. Immunol. 14:233, 1996.
Levinson et al., Nature, 295:79, 1982.
Levrero et al., Gene, 101:195-202, 1991.
Lew and Kombluth, Curr. Opin. Cell Biol., 8 795-804, 1996.
Lin et al., Mol. Cell. Biol., 10:850, 1990.
Ling et al., Mol. Pharmacol., 49:832-841, 1996.
Liu and Janeway, Proc. Natl. Acad. Sci. USA 89:3845, 1992.
Liu et al., Cell, 86:147-157, 1996.
Lock et al., Cancer Res., 54:4933-4939, 1994.
Longoni et al., Int. J. Clin. Lab. Res. 28:162, 1998.
Lowy et al., Cell, 22:817, 1980.
Luria et al., EMBO J.,6:3307, 1987.
Lusky and Botchan, Proc. Natl. Acad. Sci. USA, 83:3609, 1986.
Lusky et al., Mol. Cell. Biol., 3:1108, 1983.
Majors and Varmus, Proc. Natl. Acad. Sci. USA, 80:5866, 1983.
Manickasingham et al., J. Immunol., 161:3827, 1998.
Mann et al., Cell, 33:153-159, 1983.
Markowitz et al., J. Virol., 62:1120-1124, 1988.
Martin et al., J. Exp. Med., 182:1545-1556, 1995.
McAdam et al., Immunol. Rev. 165:231, 1998.
McNeall et al., Gene, 76:81, 1989.
Merrifield, Science, 232: 341-347, 1986.
Miksicek et al., Cell, 46:203, 1986.
Mistry et al., Anti-Cancer Drugs, 13 15-28, 2002.
Mordacq and Linzer, Genes and Dev., 3:760 1989.
Morean et al., Nucl. Acids Res., 9:6047, 1981.
Morel et al., Eur. J. Immunol., 27:26, 1997.
Morimoto and Schlossman, Immunol. Rev., 161:55, 1998.
Morimoto and Schlossman, Immunologist, 2:4, 1994.
Morimoto et al., Immunol. Rev., 161:55-70, 1998.
Morimoto et al., J. Immunol., 143:3430-3439, 1989.
Morrison et al., J. Exp. Med., 177: 1135-1143, 1993.
Morton et al., Cancer, 71:3737-3743, 1993.
Mow et al., Curr. Opin. Oncol., 13:453-462, 2001.
Muesing et al., Cell, 48:691, 1987.
Mulligan et al., Proc. Natl. Acad. Sci. USA, 78:2072, 1981.
Mulligan, Science, 260:926-932, 1993.
Nagane et al., Apoptosis, 6:191-197, 2001.
Ng et al., Nuc. Acids Res., 17:601, 1989.
Nicolas and Rubenstein, In: Vectors: A survey of molecular cloning vectors and their uses, Rodriguez and Denhardt (eds.), Stoneham: Butterworth, 493-513, 1988.
Nicolau et al., Methods Enzymol., 149:157-176, 1987.
Nilsson et al., In: Progress in Cell Cycle Research, 4:107-114, Kluwer Academic/Plenum Publishers NY, 2000.
Nishimura et al., FEBS Lett., 505:399-404, 2001.
Nykjaer et al., J. Cell Biol., 141:815, 1998.
O'Hare et al., Proc. Natl. Acad. Sci. USA, 78:1527, 1981.
Ondek et al., EMBO J., 6:1011, 1987.
Oravecz et al., J. Exp. Med., 186:1865, 1997.
Oravecz et al., J. Exp. Med., 186:1865-1872, 1997.
Ornitz et al., Mol. Cell. Biol., 7:3466, 1987.
Palmiter et al., Nature, 300:611, 1982.
Paskind et al., Virology, 67:242-248, 1975.
Pech et al., Mol. Cell. Biol. 9:396, 1989.
Peng et al., Science, 277:1501-1505, 1997.
Perez-Stable and Constantini, Mol. Cell. Biol., 10:1116, 1990.
Perkins et al., Cancer Res., 60:1645-1653, 2000.
Picard and Schaffier, Nature, 307:83, 1984.
Pinkert et al., Genes and Dev., 1:268, 1987.
Ponta et al., Proc. Natl. Acad. Sci. USA, 82:1020, 1985.
Poon et al., Cancer Res., 57:5168-5178, 1997.
Porton et al., Mol. Cell. Biol., 10:1076, 1990.
Prakken et al., Nat. Med., 6:1406, 2000.
Proost et al., J. Biol. Chem., 273:7222-7227, 1998.
Purchio et al., J. Biol. Chem., 263:14211, 1988.
Queen and Baltimore, Cell, 35:741, 1983.
Quinn et al., Mol. Cell Biol., 9:4713, 1989.
Racher et al., Biotechnology Techniques, 9:169-174, 1995.
Ragot et al., Nature, 361:647-650, 1993.
Ravindranath and Morton, Intern. Rev. Immunol., 7: 303-329, 1991.
Raynal and Pollard, Biochim. Biophys. Acta, 1197:63-93, 1994.
Redondo et al., Science, 247:1225, 1990.
Reisman and Rotter, Mol. Cell. Biol., 9:3571, 1989.
Renan, Radiother. Oncol., 19:197-218, 1990.
Resendez Jr. et al., Mol. Cell. Biol., 8:4579, 1988.
Ridgeway, In: Vectors. A survey of molecular cloning vectors and their uses, Stoneham: Butterworth, pp. 467-492, 1988.
Ripe et al., Mol. Cell. Biol., 9:2224, 1989.
Rittling et al., Nuc. Acids. Res., 17:1619, 1989.
Rosen et al., Cell, 41:813, 1988.
Rosenberg et al., Ann Surg., 210(4):474-548, 1989
Rosenfeld et al., Cell, 68:143-155, 1992.
Rosenfeld et al., Science, 252:431-434, 1991.
Roux et al., Proc. Natl. Acad. Sci. USA, 86:9079-9083 1989.
Saito, Curr. Opin. Immunol., 10:313, 1998.
Sakai et al., Genes and Dev., 2:1144, 1988.
Sambrook et al., In: Molecular cloning, Cold Spring Harbor Laboratory Press, Cold Spring Harbor, N.Y., 2001.
Samulski et al., J. Virol., 61(10):3096-3101, 1987.
Santerre et al., Gene, 30: 147-156, 1984.
Satake et al., J. Virology, 62:970, 1988.
Schaffner et al., J. Mol. Biol., 201:81, 1988.
Searle et al., Mol. Cell. Biol., 5:1480, 1985.
Sharp and Marciniak, Cell, 59:229, 1989.
Shaul and Ben-Levy, EMBO J., 6:1913, 1987.
Sherman et al., Mol. Cell. Biol., 9:50, 1989.
Siui et al., FEBS Lett., 461:299-305, 1999.
Sleigh and Lockett, J. EMBO, 4:3831, 1985.
Spalholz et al., Cell, 42:183, 1985.
Spandau and Lee, J. Virology, 62:427, 1988.
Spandidos and Wilkie, EMBO J., 2:1193, 1983.
Steinbrecher et al., J. Immunol., 166 2041-2048, 2001.
Stephens and Hentschel, Biochem. J., 248:1, 1987.
Stewart and Young, Solid Phase Peptide Synthesis, 2d. ed., Pierce Chemical Co., 1984.
Stewart et al., Anti-Cancer Drugs., 12, 359-367, 2001.
Stinchcomb et al., Nature, 282(5734):39-43, 1979
Stratford-Peiricaudet and Perricaudet, In: Human Gene Transfer, Cohen-Haguenauer and Boiron (Eds.), John Libbey Eurotext, France, 51-61, 1991.
Stratford-Perricaudet et al., Hum. Gene. Ther., 1:241-256, 1990.
Stuart et al., Nature, 317:828, 1985.
Sullivan and Peterlin, Mol. Cell. Biol., 7:3315, 1987.
Swartzendruber and Lehman, J. Cell. Physiology, 85:179, 1975.
Szybalska et al., Proc. Natl. Acad. Sci. USA, 48: 2026, 1962.
Takebe et al., Mol. Cell. Biol., 8:466, 1988.
Tam et al., J. Am. Chem. Soc., 105:6442, 1983.
Tanaka et al., Proc. Natl. Acad. Sci. USA 90:4586, 1990.
Tanaka et al., Proc. Natl. Acad. Sci. USA, 90:4586-4590, 1993.
Tanaka et al., Proc. Natl. Acad. Sci. USA, 91:3082-3086, 1994.
Tanaka et al., Int. J. Cancer, 64:326-331, 1995.
Tavernier et al., Nature, 301:634, 1983.
Taylor and Kingston, Mol. Cell. Biol., 10:165; 1990a.
Taylor and Kingston, Mol. Cell. Biol., 10:176, 1990b.
Taylor et al., J. Biol. Chem., 264:15160, 1989.
Temin, In: Gene Transfer, Kucherlapati (ed.), NY, Plenum Press, 149-188, 1986.
Thiesen et al., J. Virology, 62:614, 1988.
Top et al., J. Infect. Dis., 124:155-160, 1971.
Torimoto et al., J. Immunol., 147:2514-2517, 1991.
Torimoto et al., Mol. Immunol., 29:183-192, 1992.
Treisman, Cell, 42:889, 1985.
Tronche et al., Mol. Biol. Med., 7:173, 1990.
Trudel and Constantini, Genes and Dev. 6:954, 1987.
Tschemper et al., Gene, 10: 157, 1980.
Tur-Kaspa et al., Mol. Cell Biol., 6:716-718, 1986.
Turley et al., British J. Cancer, 75:1340-1346, 1997.
Tyndell et al., Nuc. Acids. Res., 9:6231, 1981.
Van de Velde et al., Int. Immunol. 5:1483, 1993.
Vannice and Levinson, J. Virology, 62:1305, 1988.
Vannus et al., Cell, 25:23-36, 1981.
Vasseur et al., Proc. Natl. Acad. Sci. USA, 77:1068, 1980.
Vermes et al., J. Immunol. Methods, 194:39-51, 1995.
Vicker et al., J. Med. Chem., 45:721-739, 2002.
von Bonin, et al, Immunol. Rev. 161:43-53, 1998.
Vyth-Dreese et al, Eur. J. Immunol. 25:3023, 1995.
Wagner et al., Science, 260:1510-1513, 1990.
Walunas et al., Immunity 1:4405, 1994.
Wang and Calame, Cell, 47:241, 1986.
Wang et al., J. Biol Chem., 276:3610-3619, 2001.
Wang, Ann. Rev. Biochem., 65:635-692, 1996.
Weber et al., Cell, 36:983, 1984.
Weinberger et al. Mol. Cell. Biol., 8:988, 1984.
Wen et al., Blood, 96:3900-3906, 2000.
Wigler et al., Cell, 11:223, 1977.
Wigler et al., Proc. Natl. Acad. Sci. USA, 77:3567, 1980.
Winoto and Baltimore, Cell 59:649, 1989.
Wong et al., Gene, 10:87-94, 1980.
Wu and Wu, Adv. Drug Delivery Rev., 12:159-167, 1993.
Wu and Wu, Biochemistry, 27:887-892, 1988.
Wu and Wu, J. Biol. Chem., 262:4429-4432, 1987.
Yang et al., Science, 275:1129-1132, 1997.
Yi-qun et al., Int. Immunol. 8:37, 1996.
Yokochi et al., J. Immunol. 128:823, 1982.
Yokozeki et al., J. Invest. Dermatol. 106:147, 1996.
Yutzey et al. Mol. Cell. Biol., 9:1397, 1989.

1. A method for inhibiting the growth of a cell comprising contacting the cell with:
a) a CD26 composition; and
b) a chemnotherapeutic agent and/or a radiotherapeutic agent, in dosages effective to inhibit growth of the cell.

2. The method of claim 1, wherein the CD26 composition exhibits the dipeptidyl peptidase IV (DPPIV) activity.

3. The method of claim 1, wherein the cell is simultaneously contacted with the CD26 composition and the chemotherapeutic agent and/or a radiotherapeutic agent.

4. The method of claim 1, wherein the cell is contacted with the CD26 composition prior to being contacted with the chemotherapeutic agent and/or a radiotherapeutic agent.

5. The method of claim 1, wherein the cell is contacted with the CD26 composition after being contacted with the chemotherapeutic agent and/or a radiotherapeutic agent.

6. The method of claim 1, wherein the chemotherapeutic agent is a topoisomerase II inhibitor.

7. The method of claim 6, wherein the topoisomerase II inhibitor is an anthracycline antibiotic, an amsacrine, an ellipticine, an epipodophyllotoxin, a mitoxantrone, a synthetic topoisomerase inhibitor or a derivative of any of the foregoing.

8. The method of claim 6, wherein the topoisomerase II inhibitor is doxorubicin, etoposide, daunorubicin, teniposide, dactinomycin, mitoxantrone, and/or a derivative and/or analog and/or a liposomal formulation thereof.

9. The method of claim 6, wherein the synthetic topoisomerase II inhibitor is small molecule.

10. The method of claim 1, wherein the CD26 composition is attached to a targeting agent.

11. The method of claim 10, wherein the targeting agent is an antibody, a cytokine, a receptor-ligand or any other tumor targeting molecule.

12. The method of claim 10, wherein the targeting agent is an antibody.

13. The method of claim 12, wherein the antibody is further linked to a radiotherapeutic agent, a toxin, or other cancer therapeutic agent.

14. The method of claim 1, wherein the CD26 composition comprises an expression construct comprising a DNA segment that encodes SEQ ID NO. 1; SEQ ID NO: 3,SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14,SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 36, or a fragment, isoform, mutation, or variant thereof under the control of a promoter active in the cell.

15. The method of claim 14, wherein the expression construct comprises a nucleic acid that encodes for at least the amino acid sequence Gly-Trp-Ser-Tyr-Gly (SEQ ID: NO: 42).

16. The method of claim 14, wherein the promoter is heterologous.

17. The method of claim 14, wherein the promoter is a constitutive promoter, a tissue-specific promoter, an inducible promoter, or a noninducible promoter.

18. The method of claim 14, wherein said expression construct is a viral expression construct.

19. The method of claim 18, wherein said viral expression construct is selected from the group consisting of a retrovirus, an adenovirus, an adeno-associated virus, a herpesvirus, a polyoma virus, a lentivirus, and a vaccinia virus.

20. The method of claim 14, wherein said expression construct is a non-viral expression construct.

21. The method of claim 20, wherein said non-viral expression construct is administered as a naked DNA.

22. The method of claim 20, wherein said non-viral expression construct is administered in a liposomal formulation.

23. The method of claim 1, wherein the CD26 composition is a CD26 peptide or protein that comprises an amino acid sequence of SEQ ID NO 2, SEQ ID NO: 4, SEQ ID NO: 33, SEQ ID NO: 35, SEQ ID NO: 37, or a fragment, mutation, isoform or biologically functionally equivalent thereof.

24. The method of claim 1, wherein the CD26 peptide or protein comprises an amino acid sequence that encodes at least amino acids 628-632 of SEQ ID NO 2.

25. The method of claim 1, wherein the CD26 peptide or protein is a soluble CD26 protein or peptide, a recombinantly produced CD26 peptide or protein, a CD26 fusion peptide or protein, a substantially purified CD26 peptide or protein, a partially purified CD26 peptide or protein, a naturally occurring CD26 peptide or protein, an isoform of a naturally occurring CD26 peptide or protein, or a mutant CD26 peptide or protein.

26. The method of claim 1, wherein the cell is a cancer cell.

27. The method of claim 26, wherein the cancer cell is a hematological cancer cell, a bladder cancer cell, a blood cancer cell, a breast cancer cell, a lung cancer cell, a colon cancer cell, a prostate cancer cell, a liver cancer cell, a pancreatic cancer cell, a stomach cancer cell, a testicular cancer cell, a brain cancer cell, a thyroid cancer cell, an ovarian cancer cell, a lymphatic cancer cell, a skin cancer cell, a brain cancer cell, a bone cancer cell, a soft tissue cancer cell.

28. The method of claim 1, wherein the cell is located in a human subject.

29. The method of claim 28, wherein the CD26 composition is administered systemically.

30. The method of claim 28, wherein the CD26 composition is administered by intravenous, intraarterial, intraperitoneal, intradermal, intratumoral, intramuscular, subcutaneous, intraarthricular, intrathecal, oral, dermal, nasal, buccal, rectal, or vaginal routes.

31. The method of claim 28, wherein the CD26 composition is administered locally to a tumor.

32. The method of claim 31, by direct intratumoral injection or by injection into tumor vasculature.

33. A method of inducing cell-cycle arrest in a cell comprising contacting the cell with:
a) a CD26 composition; and
b) a chemotherapeutic agent and/or a radiotherapeutic agent, in dosages effective to induce cell-cycle arrest in the cell.

34. The method of claim 33, wherein the cell is a cancer cell.

35. A method of killing a cancer cell comprising contacting the cell with:
a) a CD26 composition; and
b) a chemotherapeutic agent and/or a radiotherapeutic agent, in dosages effective to kill said cancer cell.

36. A method of potentiating the effect of a chemotherapeutic agent and/or a radiotherapeutic agent on a tumor cell comprising contacting said tumor cell with a CD26 composition and the chemotherapeutic agent and/or a radiotherapeutic agent.

37. A method of inducing apoptosis or enhancing apoptosis of a cancer cell comprising contacting the cell with:
a) a CD26 composition; and
b) a chemotherapeutic agent and/or a radiotherapeutic agent, in dosages effective to induce apoptosis or enhance apoptosis of said cancer cell.

38. A method of treating cancer in a human patient comprising administering:
a) a CD26 composition; and
b) a chemotherapeutic agent and/or a radiotherapeutic agent, to said human patient, wherein the dose of said CD26 composition, when combined with the dose of said chemotherapeutic and/or a radiotherapeutic agent, is effective to treat said cancer.

39. The method of claim 38, wherein the DNA damaging agent is a topoisomerase II inhibitor.

40. The method of claim 38, further comprising treating the patient with another anticancer agent, wherein the other anticancer agent is a therapeutic polypeptide, a nucleic acid encoding a therapeutic polypeptide, a chemotherapeutic agent, an immunotherapeutic agent, a cytokine, a chemokine, an activating agent, or a biological response modifier.

41. The method of claim 40, wherein the other anticancer agent is administered simultaneously with the CD26 composition and chemotherapeutic agent and/or a radiotherapeutic agent.

42. The method of claim 40, wherein the other anticancer agent is administered at a different time than the CD26 composition and chemotherapeutic agent and/or a radiotherapeutic agent.

43. A method of inducing tumor regression comprising administering to a patient in need thereof:
a) a CD26 composition; and
b) a chemotherapeutic agent and/or a radiotherapeutic agent, in dosages effective to cause regression of said tumor.

44. A method of inducing tumor necrosis comprising administering to a patient in need thereof:
a) a CD26 composition; and
b) a chemotherapeutic agent and/or a radiotherapeutic agent, in doses effective to induce tumor necrosis.

45. A method of treating a patient having a cancer comprising, induction of CD26 expression in cells of said cancer, and administering a chemotherapeutic and/or a radiotherapeutic agent to said patient whereby the expression of CD26 enhances the sensitivity of the cancer cell to the chemotherapeutic and/or radiotherapeutic agent.

46. The method of claim 45, wherein the chemotherapeutic is a topoisomerase II inhibitor.

47. The method of claim 45, wherein the induction of CD26 expression in cells of said cancer is by contacting the cells with a biological factor.

48. The method of claim 47, wherein the biological factor is a cytokine, a chemokine, a retinoid, an interferon, a chemotherapeutic agent, an antibody, or an antigen.

49. The method of claim 45, wherein the induction of CD26 expression in said cancer cells is by contacting the cells with a chemical agent.

50. A method for increasing topoisomerase II expression in a cell comprising contacting the cell with a CD26 composition.

51. The method of claim 50, wherein the CD26 composition comprises an expression construct comprising a DNA segment that encodes CD26 or a fragment thereof.

52. The method of claim 50, wherein the CD26 composition comprises a CD26 peptide or protein.

53. The method of claim 50, wherein said topoisomerase II is topoisomerase II α.

54. A method for activating an antigen presenting cell comprising providing to said cell a CD26 composition.

55. The method of claim 54, further comprising providing to the antigen presenting cell an antigenic composition.

56. The method of claim 55, wherein said antigenic composition comprises a tumor antigen, a bacterial antigen, a viral antigen, a fungal antigen.

57. A method for potentiating immune responses of an animal comprising activating the antigen presenting cells of said animal by providing a CD26 composition.

58. The method of claim 57, further comprising providing to the antigen presenting cells an antigenic composition.

59. The method of claim 58, wherein said antigenic composition comprises a tumor antigen, a bacterial antigen, a viral antigen, a fungal antigen.

60. The method of claim 57, wherein the CD26 composition comprises an expression construct comprising a DNA segment that encodes SEQ ID NO.1; SEQ ID NO: 3, SEQ ID NO: 5,SEQ ID NO: 6,SEQ ID NO: 7,SEQ ID NO: 8,SEQ ID NO:9,SEQ ID NO: 10, SEQ ID NO: 11, SEQ ID NO: 12, SEQ ID NO: 13, SEQ ID NO: 14, SEQ ID NO: 15, SEQ ID NO: 16,SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 34, SEQ ID NO: 36, or a fragment, isoform, mutation, or variant thereof under the control of a promoter active in the cell.

61. The method of claim 57, wherein the CD26 composition is a CD26 peptide or protein that comprises an amino acid sequence of SEQ ID NO 2, SEQ ID NO: 4, SEQ ID NO: 33, SEQ ID NO: 35, SEQ ID NO: 37, or a fragment, mutation, isoform or biologically functionally equivalent thereof

62. The method of claim 57, wherein said animal is a human.

63. The method of claim 62, wherein said human is immunosuppressed.

64. The method of claim 62, wherein said human is afflicted with cancer.

65. The method of claim 62, wherein said human is afflicted with an infection.
